SEQUENCE LISTING (136 sequences)

SEQ ID NO: 1 - protein, 116 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 2 - protein, 107 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 3 - protein, 121 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSS

SEQ ID NO: 4 - protein, 106 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIK

SEQ ID NO: 5 - protein, 712 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 6 - protein, 213 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 712 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 8 - protein, 450 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 476 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 10 - protein, 476 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 11 - protein, 683 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 221 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 13 - protein, 677 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSNIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 214 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 226 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 16 - protein, 708 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 17 - protein, 708 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 18 - protein, 446 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 679 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 673 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSNIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 452 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 233 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSS

SEQ ID NO: 23 - protein, 458 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 227 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 25 - protein, 496 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKGGGSGGGGQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 270 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGSGGGGQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSGGCGGGKVAACKEKVAALKEKVAALKEKVAALKE

SEQ ID NO: 27 - protein, 502 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGSGGGGQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 264 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKGGGSGGGGQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGCGGGKVAACKEKVAALKEKVAALKEKVAALKE

SEQ ID NO: 29 - protein, 566 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 320 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 572 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 326 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 579 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 333 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 566 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 320 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRNIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 572 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 326 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPNIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 579 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 333 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPNIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 658 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 661 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 665 aa, Artificial Sequence
  Synthetic
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 664 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 219 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 667 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 671 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGSQVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 711 aa, Artificial Sequence
  Synthetic
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 49 - protein, 218 aa, Artificial Sequence
  Synthetic
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 711 aa, Artificial Sequence
  Synthetic
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 51 - protein, 449 aa, Artificial Sequence
  Synthetic
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 481 aa, Artificial Sequence
  Synthetic
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 53 - protein, 481 aa, Artificial Sequence
  Synthetic
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 54 - protein, 106 aa, Artificial Sequence
  Synthetic
DIQMTQSPSSLSASVGDRVTITCKASQNVYKKLEWYQQKPGKVPKVLIYHTNILQTGVPSRFSGSGSGTDYTLTISSLQPEDVATYYCYQWNSGPTFGGGTKVEIK

SEQ ID NO: 55 - protein, 122 aa, Artificial Sequence
  Synthetic
QVQLVQSGAEVKKPGASVKVSCKASGYTFTDFWMSWVRQAPGQGLEWMGQIYPNTGTTHSNEKFKGRVTMTRDTSTSTVYMELSSLRSEDTAVYYCARSYHISTTPNWFAYWGQGTLVTVSS

SEQ ID NO: 56 - protein, 713 aa, Artificial Sequence
  Synthetic
QVQLVQSGAEVKKPGASVKVSCKASGYTFTDFWMSWVRQAPGQGLEWMGQIYPNTGTTHSNEKFKGRVTMTRDTSTSTVYMELSSLRSEDTAVYYCARSYHISTTPNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 57 - protein, 213 aa, Artificial Sequence
  Synthetic
DIQMTQSPSSLSASVGDRVTITCKASQNVYKKLEWYQQKPGKVPKVLIYHTNILQTGVPSRFSGSGSGTDYTLTISSLQPEDVATYYCYQWNSGPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 713 aa, Artificial Sequence
  Synthetic
QVQLVQSGAEVKKPGASVKVSCKASGYTFTDFWMSWVRQAPGQGLEWMGQIYPNTGTTHSNEKFKGRVTMTRDTSTSTVYMELSSLRSEDTAVYYCARSYHISTTPNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 59 - protein, 450 aa, Artificial Sequence
  Synthetic
QVQLVQSGAEVKKPGASVKVSCKASGYTFTDFWMSWVRQAPGQGLEWMGQIYPNTGTTHSNEKFKGRVTMTRDTSTSTVYMELSSLRSEDTAVYYCARSYHISTTPNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 476 aa, Artificial Sequence
  Synthetic
DIQMTQSPSSLSASVGDRVTITCKASQNVYKKLEWYQQKPGKVPKVLIYHTNILQTGVPSRFSGSGSGTDYTLTISSLQPEDVATYYCYQWNSGPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 61 - protein, 476 aa, Artificial Sequence
  Synthetic
DIQMTQSPSSLSASVGDRVTITCKASQNVYKKLEWYQQKPGKVPKVLIYHTNILQTGVPSRFSGSGSGTDYTLTISSLQPEDVATYYCYQWNSGPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 62 - protein, 121 aa, Artificial Sequence
  Synthetic
QVQLKESGPGLVQPSETLSLTCNVSGFSLTSYNVHWVRQPPGKGLEWLGVIWSGVRTDYNSVLKSRLSIRWDSSKNQVFLKMNSLQSEDTATYYCARGTYDANYHDVMDAWGQGASVTVSS

SEQ ID NO: 63 - protein, 106 aa, Artificial Sequence
  Synthetic
DTVLTQSPALAVSPGERVTISCRASKSVRTGMHWYQQKPGQQPKLLVYGASNLESGVPARFSGSGSGTDFTLTIDPVEADDIATYFCHQSWNHFTFGSGTKLEIKR

SEQ ID NO: 64 - protein, 121 aa, Artificial Sequence
  Synthetic
QVQLKESGPGLVQPSETLSLTCTVSGFSLTSYNVHWVRQPPGKGLEWLGVIWSGVRTDYNSVLKPRLSISRDSSKNKVFLNMNSLQSEDTATYYCARGTYDDNYHDVMDAWGQGASVTVSS

SEQ ID NO: 65 - protein, 106 aa, Artificial Sequence
  Synthetic
DTVLTQSPALAVSPGERVTISCRASKSVRTGMHWYQQKPGQQPKLLIYGASNLESGVPVRFSGSGSGTDFTLTIDPVEADDTATYFCQQSWNHFTFGSGTKLEIKR

SEQ ID NO: 66 - protein, 162 aa, Artificial Sequence
  Synthetic
LPDGVLSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARDQGYTYGGDAFDVWGQGTMVTVSSASTKGPSVFPLAPSSKSTSGGTTALGCLVKDYFP

SEQ ID NO: 67 - protein, 160 aa, Artificial Sequence
  Synthetic
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWRTFGQGTKLEIKRTVAAPSVFIFPPSDEQLKSGTASVVCLLNNFYP

SEQ ID NO: 68 - protein, 121 aa, Artificial Sequence
  Synthetic
QVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSS

SEQ ID NO: 69 - protein, 107 aa, Artificial Sequence
  Synthetic
EIVLTQSPATLSLSPGERATLSCRASKSVRTGMHWYQQKPGQAPRLLIYGASNLESGIPARFSGSGSGTDFTLTISSLEPEDFAVYYCHQSWNHFTFGQGTKLEIKR

SEQ ID NO: 70 - protein, 222 aa, Artificial Sequence
  Synthetic
QVQLQESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTTVTVSSDIVLTQSPASLSASVGETVTITCRASGNIHNYLAWYQQKQGKSPQLLVYYTTTLADGVPSRFSGSGSGTQYSLKINSLQPDDFGSYYCQHFWSTPTFGGGTKLEIK

SEQ ID NO: 71 - protein, 227 aa, Artificial Sequence
  Synthetic
QVQLKESGPGLVQPSETLSLTCNVSGFSLTSYNVHWVRQPPGKGLEWLGVIWSGVRTDYNSVLKSRLSIRWDSSKNQVFLKMNSLQSEDTATYYCARGTYDANYHDVMDAWGQGASVTVSSDTVLTQSPALAVSPGERVTISCRASKSVRTGMHWYQQKPGQQPKLLVYGASNLESGVPARFSGSGSGTDFTLTIDPVEADDIATYFCHQSWNHFTFGSGTKLEIKR

SEQ ID NO: 72 - protein, 107 aa, Artificial Sequence
  Synthetic
DTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIKR

SEQ ID NO: 73 - protein, 448 aa, Artificial Sequence
  Synthetic
QVQLKESGPGLVQPSETLSLTCNVSGFSLTSYNVHWVRQPPGKGLEWLGVIWSGVRTDYNSVLKSRLSIRWDSSKNQVFLKMNSLQSEDTATYYCARGTYDANYHDVMDAWGQGASVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 212 aa, Artificial Sequence
  Synthetic
DTVLTQSPALAVSPGERVTISCRASKSVRTGMHWYQQKPGQQPKLLVYGASNLESGVPARFSGSGSGTDFTLTIDPVEADDIATYFCHQSWNHFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 450 aa, Artificial Sequence
  Synthetic
QVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 213 aa, Artificial Sequence
  Synthetic
EIVLTQSPATLSLSPGERATLSCRASKSVRTGMHWYQQKPGQAPRLLIYGASNLESGIPARFSGSGSGTDFTLTISSLEPEDFAVYYCHQSWNHFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 213 aa, Artificial Sequence
  Synthetic
DTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 161 aa, Artificial Sequence
  Synthetic
MKHLWFFLLLVAAPRWVLSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGRHDYGDYFDYWGQGTLVTVSSGSASAPTLFPLVSCENSPSDTSK

SEQ ID NO: 79 - protein, 160 aa, Artificial Sequence; identical to SEQ ID NO: 67
  Synthetic

SEQ ID NO: 80 - protein, 121 aa, Artificial Sequence
  Synthetic
QVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSS

SEQ ID NO: 81 - protein, 107 aa, Artificial Sequence
  Synthetic
EIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIKR

SEQ ID NO: 82 - protein, 448 aa, Artificial Sequence
  Synthetic
QVQLKESGPGLVQPSETLSLTCTVSGFSLTSYNVHWVRQPPGKGLEWLGVIWSGVRTDYNSVLKPRLSISRDSSKNKVFLNMNSLQSEDTATYYCARGTYDDNYHDVMDAWGQGASVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 212 aa, Artificial Sequence
  Synthetic
DTVLTQSPALAVSPGERVTISCRASKSVRTGMHWYQQKPGQQPKLLIYGASNLESGVPVRFSGSGSGTDFTLTIDPVEADDTATYFCQQSWNHFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 450 aa, Artificial Sequence
  Synthetic
QVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 213 aa, Artificial Sequence
  Synthetic
EIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 711 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIKGGGGSGGGGSGGGGSGGGGSQVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSS

SEQ ID NO: 87 - protein, 711 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSSGGGGSGGGGSGGGGSGGGGSDTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIK

SEQ ID NO: 88 - protein, 445 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 481 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIKGGGGSGGGGSGGGGSGGGGSQVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSS

SEQ ID NO: 90 - protein, 481 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSSGGGGSGGGGSGGGGSGGGGSDTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIK

SEQ ID NO: 91 - protein, 712 aa, Artificial Sequence
  Synthetic
QVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 92 - protein, 712 aa, Artificial Sequence
  Synthetic
QVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 93 - protein, 450 aa, Artificial Sequence
  Synthetic
QVQLQESGPGLVKPSETLSLTCTVSGFSLTSYNVHWIRQPPGKGLEWIGVIWSGVRTDYNSVLKSRVTISVDTSKNQFSLKLSSVTAADTAVYYCARGTYDANYHDVMDAWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 476 aa, Artificial Sequence
  Synthetic
DTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 95 - protein, 476 aa, Artificial Sequence
  Synthetic
DTVLTQSPATLSLSPGERATLSCRASKSVRTGMHWYQQKPGQAPRLLVYGASNLESGIPARFSGSGSGTDFTLTISSLEPEDFATYFCHQSWNHFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 96 - protein, 711 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIKGGGGSGGGGSGGGGSGGGGSQVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSS

SEQ ID NO: 97 - protein, 711 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSSGGGGSGGGGSGGGGSGGGGSEIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIK

SEQ ID NO: 98 - protein, 481 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIKGGGGSGGGGSGGGGSGGGGSQVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSS

SEQ ID NO: 99 - protein, 481 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSSGGGGSGGGGSGGGGSGGGGSEIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIK

SEQ ID NO: 100 - protein, 712 aa, Artificial Sequence
  Synthetic
QVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 101 - protein, 712 aa, Artificial Sequence
  Synthetic
QVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 102 - protein, 450 aa, Artificial Sequence
  Synthetic
QVQLQESGPGLVKPSETLSLTCTVSGFSLTSYNVHWIRQPPGKGLEWIGVIWSGVRTDYNSVLKPRVTISVDTSKNQFSLKLSSVTAADTAVYYCARGTYDDNYHDVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 476 aa, Artificial Sequence
  Synthetic
EIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 104 - protein, 476 aa, Artificial Sequence
  Synthetic
EIVLTQSPATLSLSPGERATLSCRASKSVRTGMHWYQQKPGQAPRLLIYGASNLESGIPARFSGSGSGTDFTLTISSLEPEDFAVYYCQQSWNHFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 105 - protein, 451 aa, Artificial Sequence
  Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 106 - protein, 214 aa, Artificial Sequence
  Synthetic
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 717 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 108 - protein, 717 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 109 - protein, 487 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 110 - protein, 487 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 111 - protein, 717 aa, Artificial Sequence
  Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 112 - protein, 214 aa, Artificial Sequence; identical to SEQ ID NO: 106
  Synthetic

SEQ ID NO: 113 - protein, 717 aa, Artificial Sequence
  Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 114 - protein, 455 aa, Artificial Sequence
  Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 477 aa, Artificial Sequence
  Synthetic
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 116 - protein, 477 aa, Artificial Sequence
  Synthetic
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 117 - protein, 447 aa, Artificial Sequence
  Synthetic
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 214 aa, Artificial Sequence
  Synthetic
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 708 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKGGGGSGGGGSGGGGSGGGGSEVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 120 - protein, 708 aa, Artificial Sequence
  Synthetic
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSGGGGSGGGGSGGGGSGGGGSDIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 121 - protein, 478 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKGGGGSGGGGSGGGGSGGGGSEVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 122 - protein, 478 aa, Artificial Sequence
  Synthetic
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSGGGGSGGGGSGGGGSGGGGSDIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 123 - protein, 708 aa, Artificial Sequence
  Synthetic
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 124 - protein, 224 aa, Artificial Sequence
  Synthetic
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 125 - protein, 446 aa, Artificial Sequence
  Synthetic
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 477 aa, Artificial Sequence
  Synthetic
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 127 - protein, 477 aa, Artificial Sequence
  Synthetic
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 128 - protein, 5 aa, Artificial Sequence
  Synthetic
GGGGS

SEQ ID NO: 129 - protein, 10 aa, Artificial Sequence
  Synthetic
GGGGSGGGGS

SEQ ID NO: 130 - protein, 15 aa, Artificial Sequence
  Synthetic
GGGGSGGGGSGGGGS

SEQ ID NO: 131 - protein, 20 aa, Artificial Sequence
  Synthetic
GGGGSGGGGSGGGGSGGGGS

SEQ ID NO: 132 - protein, 25 aa, Artificial Sequence
  Synthetic
GGGGSGGGGSGGGGSGGGGSGGGGS

SEQ ID NO: 133 - protein, 30 aa, Artificial Sequence
  Synthetic
GGGGSGGGGSGGGGSGGGGSGGGGSGGGGS

SEQ ID NO: 134 - protein, 5 aa, Artificial Sequence
  Synthetic
SGGGS

SEQ ID NO: 135 - protein, 20 aa, Artificial Sequence
  Synthetic
GGGGSGGGGSGGGGSGGGGS

SEQ ID NO: 136 - protein, 708 aa, Artificial Sequence
  Synthetic
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSSGGGGSGGGGSGGGGSGGGGSDIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

(19) United States
(12) Patent Application Publication
(10) Pub. No.: US 2023/0406950 A1
(43) Pub. Date: Dec. 21, 2023

(54) BI-SPECIFIC ANTIBODIES COMPRISING ANTI-CD137 BINDING MOLECULES

(71) Applicant: LYVGEN BIOPHARMA HOLDINGS LIMITED, Grand Cayman (KY)

(72) Inventors: Jieyi WANG, Belmont, CA (US); Yi WU, Shanghai (CN)

(73) Assignee: LYVGEN BIOPHARMA HOLDINGS LIMITED, Grand Cayman (KY)

(21) Appl. No.: 18/252,359

(22) PCT Filed: Nov. 10, 2021
(86) PCT No.: PCT/US21/58693

§ 371 Date: May 9, 2023

(30) Foreign Application Priority Data
Nov. 10, 2020 (WO) PCT/CN2020/127890

(51) Int. Cl. C07K 16/28; A61P 35/00

(52) U.S. Cl. CPC C07K 16/2878; C07K 16/2818; C07K 16/2827; A61P 35/00; C07K 2317/31; C07K 2317/622; C07K 2317/565; C07K 2317/24; A61K 2039/505

(57) ABSTRACT

Provided herein are antibodies binding to CD137 and bi-specific antibodies comprising such for targeting both CD137 and a second suitable antigen such as an immune checkpoint or modulator molecule. Examples include PD-1, PD-L1, GITR, CD40, or OX40. Also provided herein are therapeutic uses of such antibodies.

### CROSS REFERENCE TO RELATED APPLICATIONS

This application claims the benefit of the filing dates of International Patent Application No.: PCT/CN2020/127890, filed Nov. 10, 2020, the entire contents of which are incorporated by reference herein.

### BACKGROUND OF THE INVENTION

Human tumor necrosis factor receptor (TNFR) superfamily member 9 (TNFRSF9, also known as CD137 or 4-1BB) is expressed by activated T cells and other lymphoid cells (e.g., dendritic cells, B cells, natural killer cells and granulocytes. Upon activation, CD137 signaling promotes cytokine release and cytotoxic activity, and prevents activation-induced cell death. In the tumor microenvironment, enriched CD137 expression has been observed on tumor reactive T cells and tumor vessels.

Agonistic anti-CD137 antibodies can mimic the activity of the natural ligand of CD137 and enhance the functions of tumor-infiltrating, cytolytic CD8+ T cells, which play a major role in anti-cancer effects. However, in some instances, such therapeutic approaches failed to achieve desired clinical efficacy and/or raised safety concerns. It is therefore of great interest to develop new CD137-targeting immune therapies that are effective and safe.

### SUMMARY OF THE INVENTION

The present disclosure is based, at least in part, on the development of bi-specific antibodies targeting both human CD137 and a second desired antigen, such as PD-1, PD-L1, GITR, CD40, or OX40. Such bi-specific antibodies exhibit substantially similar antigen-binding affinity and specificity as the parent antibodies and show one or more superior features, for example, simultaneous binding to both target antigens, enhanced agonistic activity of CD137 and optionally of the second desired antigen, superior anti-tumor activities, or a combination thereof.

The bi-specific antibodies disclosed herein show superior anti-tumor activities in animal models relative to their corresponding parent or representative approved antibody therapeutics, either alone or in combined therapy. For example, the exemplary anti-PD-1/CD137 bi-specific (bsAb) clones Ly457, Ly458 and Ly459, and the exemplary anti-GITR/CD137 bsAb clone Ly754 exhibited superior anti-tumor activities than their parent antibodies or representative approved antibody therapeutics, either taken alone or in combination. In addition, the exemplary anti-GITR/CD137 bsAb clones Ly746 and Ly749 showed higher T cells stimulation activities than the combination of their anti-GITR and anti-CD137 parental mAbs.

Accordingly, some aspects of the present disclosure features a bi-specific antibody, comprising: (a) a first antibody moiety that binds human CD137, and (b) a second antibody moiety that binds a desired antigen. In some examples, the desired antigen is PD-1. In some examples, the desired antigen is PD-L1. In some examples, the desired antigen is GITR. In some examples, the desired antigen is CD40. In some examples, the desired antigen is OX40.

In some embodiments, the first antibody moiety is in a single-chain antibody (scFv) format and optionally the second antibody moiety is in a full-length antibody format comprising a heavy chain and a light chain. Alternatively, the second antibody moiety is in a scFv format and optionally the first antibody moiety is in a full-length antibody format comprising a heavy chain and a light chain.

In some examples, the first antibody moiety that binds human CD137 is a scFv; and the second antibody moiety comprises a first polypeptide comprising an antibody heavy chain and a second polypeptide comprising an antibody light chain. The scFv may be fused to the first polypeptide. Alternatively, the scFv may be fused to the second polypeptide.

In some examples, the second antibody moiety that binds PD-1, PD-L1, GITR, CD40 or OX40 is a scFv and the first antibody moiety that binds human CD137 comprises a first polypeptide comprising an antibody heavy chain and a second polypeptide comprising an antibody light chain. The scFv may be fused to the first polypeptide. Alternatively, the scFv may be fused to the second polypeptide.

In some embodiments, any of the bi-specific antibodies disclosed herein may be in a three-chain format. In some examples, such a bi-specific antibody may comprise: (i) a first polypeptide, which comprises a heavy chain of the first antibody moiety fused to a light chain of the second antibody moiety; (ii) a second polypeptide, which comprises a light chain of the first antibody moiety; and (iii) a third polypeptide, which comprises a heavy chain of the second antibody moiety. In some instances, the heavy chain of the second antibody moiety may comprise a V_{H} and a heavy chain constant domain, which optionally is CH1. In other examples, such a bi-specific antibody may comprise: (i) a first polypeptide, which comprises a heavy chain of the second antibody moiety fused to a light chain of the first antibody moiety; (ii) a second polypeptide, which comprises a light chain of the second antibody moiety; and (iii) a third polypeptide, which comprises a heavy chain of the first antibody moiety. In some instances, the heavy chain of the first antibody moiety comprises a V_{H} and a heavy chain constant domain, which optionally is CH1.

In any of the bi-specific antibodies disclosed herein, the first antibody moiety that binds human CD137 may have the same heavy chain and light chain CDRs as reference antibody Ly1630. In some examples, the first antibody moiety that binds human CD137 may comprise the same V_{H} and/or V_{L} as reference antibody Ly1630.

In some embodiments, the second antibody moiety in any of the bi-specific antibodies disclosed herein may bind PD-1. For example, the second antibody moiety that binds PD-1 may comprise the same heavy chain CDRs as reference antibody Ly516, and/or the same light chain CDRs as reference antibody Ly516. In some examples, the second antibody moiety that binds PD-1 may comprise the same V_{H} and/or V_{L} as reference antibody Ly516. Exemplary anti-CD137/PD-1 bi-specific antibodies include Ly456, Ly457, Ly458, Ly459, Ly460, Ly461, Ly510, Ly511, Ly512, Ly513, Ly514, Ly515, Ly555, Ly556, Ly557, Ly558, Ly666, Ly667, Ly668, Ly669, Ly670, Ly671, Ly672, Ly673, Ly674, Ly675, Ly676, Ly677, Ly712, Ly713, Ly714 and Ly715.

In some embodiments, the second antibody moiety in any of the bi-specific antibodies disclosed herein may bind PD-L1. For example, the second antibody moiety comprises the same heavy chain CDRs as reference antibody Ly076, and/or the same light chain CDRs as reference antibody Ly076. In some examples, the second antibody moiety that binds PD-L1 may comprise the same V_{H} and/or V_{L} as reference antibody Ly076. Exemplary anti-CD137/PD-L1 bi-specific antibodies include Ly299, Ly346, Ly347, and Ly348.

In some embodiments, the second antibody moiety in any of the bi-specific antibodies disclosed herein may bind GITR. In some instances, the second antibody moiety that binds GITR may comprise the same heavy chain CDRs as reference antibody Lyv392 and/or the same light chain CDRs as reference antibody Lyv392. In some examples, the second antibody moiety that binds GITR may comprise the same V_{H} and/or V_{L} as reference antibody Lyv392.

In other instances, the second antibody moiety that binds GITR may comprise the same heavy chain CDRs as reference antibody Lyv396 and/or the same light chain CDRs as reference antibody Lyv396. In some examples, the second antibody moiety that binds GITR may comprise the same V_{H} and/or V_{L} as reference antibody Lyv396. Exemplary anti-CD137/GITR bi-specific antibodies include Ly746, Ly747, Ly748, Ly749, Ly750, Ly751, Ly752, Ly753, Ly754, Ly755, Ly756, Ly757, Ly758, Ly759, Ly760, Ly761, Ly1523, Ly1524, Ly1525, and Ly1526.

In some embodiments, the second antibody moiety in any of the bi-specific antibodies disclosed herein may bind CD40. For example, the second antibody moiety comprises the same heavy chain CDRs as reference antibody Ly253, and/or the same light chain CDRs as reference antibody Ly253. In some examples, the second antibody moiety that binds CD40 may comprise the same V_{H} and/or V_{L} as reference antibody Ly253. Exemplary anti-CD137/CD40 bi-specific antibodies include Ly738, Ly739, Ly740, Ly741, Ly742, Ly743, Ly744, and Ly745.

In some embodiments, the second antibody moiety in any of the bi-specific antibodies disclosed herein may bind OX40. For example, the second antibody moiety comprises the same heavy chain CDRs as reference antibody Ly598, and/or the same light chain CDRs as reference antibody Ly598. In some examples, the second antibody moiety that binds CD40 may comprise the same V_{H} and/or V_{L} as reference antibody Ly598. Exemplary anti-CD137/OX40 bi-specific antibodies include Ly762, Ly763, Ly764, Ly765, Ly766, Ly767, Ly768, Ly769, Ly1519, Ly1520, Ly1521, and Ly1522.

In another aspect, the present disclosure provides an isolated antibody specific to human glucocorticoid-induced TNFR-related protein (GITR) (anti-GITR antibody), wherein the anti-GITR antibody comprises: (a) a heavy chain variable region (V_{H}) comprising heavy chain complementary determining regions (CDRs) 1, 2, and 3, which are either identical to those of a reference antibody, which is Lyv392 or Lyv396, or contain no more than five amino acid residue variations relative to the reference antibody; and (b) a light chain variable region (V_{L}), comprising light chain complementary determining regions (CDRs) 1, 2, and 3, which are either identical to those of the reference antibody or contain no more than five amino acid residue variations relative to the reference antibody. In some examples, the reference antibody is Lyv392. In other examples, the reference antibody is Lyv396.

Any of the anti-GITR antibodies disclosed herein may be a humanized antibody comprising a human V_{H} framework and a human V_{L} framework. In some examples, the human V_{H} framework region is from IGHV4-59*01, and/or the human V_{L} framework is from IGKV3-11*01. In some instances, either the heavy chain framework region or the light chain framework region, or both include one or more mutations relative to the corresponding germline framework. In some examples, the V_{L} may comprise one or more mutations in the human V_{H} framework. For example, the one or more mutations in the V_{L} framework are back mutations based on amino acid residues in the reference antibody Lyv392 at corresponding positions. In specific examples, the one or more back mutations comprise E1D, I2T, I48V, V85T, Y87F, or a combination thereof. Such a humanized V_{L} chain may comprise the amino acid sequence of SEQ ID NO:69, SEQ ID NO:72, or SEQ ID NO:81. Alternatively or in addition, the V_{H} comprises the amino acid sequence of SEQ ID NO:68 or SEQ ID NO:80.

In some examples, the anti-GITR antibody disclosed herein may comprise: a V_{H} chain comprising the amino acid sequence of SEQ ID NO:68 and a V_{L} chain comprising the amino acid sequence of SEQ ID NO:69. In some examples, the anti-GITR antibody disclosed herein may comprise the amino acid sequence of SEQ ID NO:68 and a V_{L} chain comprising the amino acid sequence of SEQ ID NO:72. In some examples, the anti-GITR antibody may comprise the amino acid sequence of SEQ ID NO:80 and a V_{L} chain comprising the amino acid sequence of SEQ ID NO:81.

Any of the anti-GITR antibodies disclosed herein may be a full-length antibody. In some examples, the full-length antibody is an IgG/kappa molecule. In specific examples, the full-length antibody may comprise a heavy chain that is an IgG1, IgG2, or IgG4 chain. In some instances, the heavy chain may comprise a mutated Fc region, which exhibits altered binding affinity or selectivity to an Fc receptor. Examples of such anti-GITR antibodies include TM676, TM677, or TM685.

In another aspect, provided herein is a nucleic acid or a nucleic acid set, which collectively encodes any of the bi-specific antibodies or any of the anti-GITR antibodies disclosed herein. In some embodiments, the nucleic acid or nucleic acid set, which is an expression vector or an expression vector set. Also within the scope of the present disclosure is a host cell, comprising the nucleic acid or nucleic acid set coding for any of the antibodies disclosed herein. In some examples, the host cell is a mammalian host cell.

Further, provided herein is a method for producing any of the bi-specific antibodies or anti-GITR antibodies disclosed herein, the method comprising: (i) culturing the host cell of claim C3 or claim C4 under conditions allowing for expression of the antibody; and (ii) harvesting the antibody thus produced.

In addition, the present disclosure provides a pharmaceutical composition, comprising an antibody or bi-specific antibody set forth here, or a nucleic acid(s) encoding such, and a pharmaceutically acceptable carrier.

Also within the scope of the present disclosure is a method for modulating immune responses, comprising administering an effective amount of the antibody of any one of bi-specific antibodies or anti-GITR antibodies, a nucleic acid(s) encoding such, or a pharmaceutical composition comprising the antibody or encoding nucleic acid(s), to a subject in need thereof. In some examples, the subject is a human patient having or suspected of having cancer. Further, provided herein are pharmaceutical compositions comprising any of the antibodies disclosed herein or coding nucleic acids thereof for use in treating the target diseases disclosed herein or uses of such antibodies or coding nucleic acids for manufacturing medicaments for the intended medical uses as also disclosed herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1B are charts showing PD-1 binding activity of anti-PD-1/CD137 bispecific antibodies as indicated to human PD-1 expressed on CHO cells. Binding of these anti-PD-1/CD137 bispecific antibodies are indicated by the mean fluorescence intensity (MFI). 1A: Clones Ly456, Ly457, Ly458, Ly459, Ly460, Ly461, Ly510, Ly511, Ly514, Ly515, Ly516v and Ly1630 at various concentrations as indicated. 1B: Clones Ly512, Ly513, Ly516v and Ly1630 at various concentrations as indicated.

FIGS. 2A-2B are charts showing CD137 binding activity of exemplary anti-PD-1/CD137 bispecific antibodies as indicated to human CD137 expressed on CHO cells. Binding of these anti-PD-1/CD137 bispecific antibodies are indicated by the mean fluorescence intensity (MFI). 2A: Clones Ly456, Ly457, Ly458, Ly459, Ly460, Ly461, Ly510, Ly511, Ly514, Ly515, Ly1630 and Ly516v at various concentrations as indicated. 2B: Clones Ly512, Ly513, Ly1630 and Ly516v at various concentrations as indicated.

FIGS. 3A-3J are a set of graphs showing simultaneously binding of exemplary anti-PD-1/CD137 antibodies to recombinant human PD-1 and CD137 proteins. Clones Ly456 (3A), Ly457 (3B), Ly458 (3C), Ly459 (3D), Ly460 (3E), Ly510 (3F), Ly511 (3G), Ly512 (3H), Ly513 (3I) and Ly514 (3J) at various concentrations as indicated.

FIG. 4 is a chart showing stimulation of human CD137 activation as indicated by IL8 secretion in a reporter assay by a number of anti-PD-1/CD137 antibodies. The agonistic activity was evaluated when these bispecific antibodies were co-cultured with PD-1 overexpressing cells. The bars labeled as “IgG control” and “Mediun” served as controls.

FIG. 5 is a chart showing the PD-1 pathway blocking effect of anti-PD-1/CD137 bispecific antibodies co-cultured with CD137 overexpressing CHO cells. The antibodies are as indicated, and the RLU signal reflects the blockade of PD-1/PD-L1 interaction leading to increased signal.

FIG. 6 is a chart showing the stimulation activity of exemplary anti-PD-1/CD137 bispecific antibodies at the concentration of 3 μg/mL on the SEB-activated human PBMC cells from one healthy donor. The various antibodies are as indicated and the stimulation of human PBMC cells are indicated by the secretion of IL-2.

FIGS. 7A-7J include a set of graphs showings pharmacokinetics of anti-PD-1/CD137 bispecific antibodies as indicated in mice. Exemplary clones include Ly456 (7A), Ly457 (7B), Ly458 (7C), Ly459 (7D), Ly460 (7E), Ly510 (7F), Ly511 (7G), Ly512 (7H), Ly513 (7I) and Ly514 (7J).

FIGS. 8A-8C are a set of graphs showing the anti-tumor activity of anti-PD-1/CD137 antibodies in a human CD137 and human PD-1 double knock-in mouse syngeneic model with different human tumor cells. 8A: anti-tumor effects in MC38-hPD-L1 model of clones Ly456, Ly457, Ly458, Ly459, Ly510, Ly511, Ly512, Ly513, Ly516v and Ly1630 at 5 mg/kg administered on day 0, 20 and 27 by intraperitoneal injection. 8B: anti-tumor effects in B16-OVA model of clones Ly457, Ly458, Ly459 and Keytruda at doses as shown administered on day 0 by intraperitoneal injection. 8C: anti-tumor effects in B16-OVA model of clones Ly457, Ly1630 and Keytruda at doses as shown administered on day 6 by intraperitoneal injection.

FIGS. 9A-9B include diagrams showing binding activity of exemplary bi-specific antibodies. FIG. 9A: a chart showing binding activity of anti-PD-L1/CD137 antibodies as indicated to human PD-L1 expressed on CHO cells. The bars (“IgG control” and “2nd”) served as controls. Binding is indicated by the mean fluorescence intensity (MFI). Clones Ly346, Ly347, Ly348, Ly299, Ly1630 and Ly076 at various concentrations as indicated. FIG. 9B: a chart showing binding activity of anti-PD-L1/CD137 bi-specific antibodies as indicated to human CD137 expressed on CHO cells. The bars (“IgG control” and “2nd”) served as controls. Binding is indicated by the mean fluorescence intensity (MFI). Clones Ly346, Ly347, Ly348, Ly299, Ly1630 and Ly076 at various concentrations as indicated were examined.

FIGS. 10A-10D are charts showing simultaneously binding of exemplary anti-PD-L1/CD137 antibodies to recombinant human PD-L1 and CD137 proteins. 10A: Clones Ly347 at various concentrations as indicated. 10B: Clones Ly299 at various concentrations as indicated. 10C: Clones Ly348 at various concentrations as indicated. 10D: Clones Ly346 at various concentrations as indicated.

FIG. 11 is a chart showing stimulation of human CD137 activation as indicated by IL8 secretion in a reporter assay by a number of anti-PD-L1/CD137 bi-specific antibodies.

FIGS. 12A-12B are charts showing the stimulation activity of exemplary anti-PD-1/CD137 bispecific antibodies on the OKT3 (2 μg/ml)-activated human PBMC cells from two healthy donors. PBMC were co-cultured with human PD-L1 over-expressing CHO cells (1×10⁴ cell/well). The various antibodies are as indicated and the stimulation of human PBMC cells are indicated by the secretion of IL-2. 12A: PBMC from donor 1 stimulated with Clones Ly346, Ly347, Ly348, Ly299, Ly076 and Ly1630. 12B: PBMC from donor 2 stimulated with Clones Ly346, Ly347, Ly348, Ly299, Ly076 and Ly1630.

FIGS. 13A-13D include a set of graphs showings pharmacokinetics of anti-PD-L1/CD137 bispecific antibodies as indicated in mice. FIG. 13A: Clone Ly346. FIG. 13B: Clone Ly347. FIG. 13C: Clone Ly348. FIG. 13D: Clone Ly299.

FIGS. 14A-14B are charts showing binding activity of anti-GITR antibodies as indicated to human GITR expressed on CHO cells. Binding of these anti-GITR antibodies are indicated by the mean fluorescence intensity (MFI). 14A: Clones TM392, TM396 and TM685 14B: Clones TM676 and TM677 at various concentrations as indicated.

FIGS. 15A-15B are charts showing stimulation of human GITR activation as indicated by IL8 secretion in a reporter assay by a number of anti-GITR antibodies. 15A: Clones TM677, TM685 and TM392. 15B: Clones TM685 and TM396 at various concentrations as indicated.

FIG. 16 is a chart showing anti-tumor activities of humanized anti-GITR antibodies. Anti-tumor effects of clones TM676, TM677 and TM685 FIGS. 17A-17B are charts showing GITR binding activity of exemplary anti-GITR/CD137 bispecific antibodies as indicated to human GITR expressed on CHO cells. The bars labeled “IgG control” served as controls. Binding of these anti-GITR/CD137 bispecific antibodies are indicated by the mean fluorescence intensity (MFI). 17A: Clones Ly748, Ly750, Ly751, Ly754, Ly755, Ly756, TM677 and TM685 at various concentrations as indicated. 17B: Clones Ly757, Ly760, TM677 and TM685 at various concentrations as indicated.

FIGS. 18A-18B are charts showing CD137 binding activity of exemplary anti-GITR/CD137 bispecific antibodies as indicated to human CD137 expressed on CHO cells. Ly076 was used as controls. Binding of these anti-GITR/CD137 bispecific antibodies are indicated by the mean fluorescence intensity (MFI). 18A: Clones Ly750, Ly751, Ly752, Ly753, Ly754 and Ly1630 at various concentrations as indicated. 18B: Clones Ly756, Ly757, Ly758, Ly759, Ly760, Ly761 and Ly1630 at various concentrations as indicated.

FIGS. 19A-19B are charts showing stimulation of human CD137 activation as indicated by IL8 secretion in a reporter assay by a number of anti-GITR/CD137 antibodies. The agonistic activity of these bispecific antibodies was evaluated in presence of GITR overexpressing CHO cells. The bar labeled as “Mediun” served as control. 19A: Clones Ly754, Ly755, Ly756, Ly757, Ly758, Ly759, Ly760, Ly761, Ly1630, TM677 and TM685 at various concentrations as indicated. 19B: Clones Ly746, Ly747, Ly748, Ly749, Ly750, Ly751, Ly752, Ly753, Ly1630, TM677 and TM685 at various concentrations as indicated.

FIGS. 20A-20B are charts showing stimulation of human GITR activation as indicated by IL8 secretion in a reporter assay by a number of anti-GITR/CD137 antibodies. The agonistic activity of these bispecific antibodies was evaluated in presence of CD137 overexpressing CHO cells. The bar labeled as “Mediun” served as control. 20A: Clones Ly746, Ly747, Ly748, Ly749, Ly750, Ly751, Ly752, Ly753, Ly1630 and TM677 at various concentrations as indicated. 20B: Clones Ly754, Ly755, Ly756, Ly757, Ly758, Ly759, Ly760, Ly761, Ly1630 and TM685 at various concentrations as indicated.

FIGS. 21A-21B are charts showing the stimulation activity of exemplary anti-GITR/CD137 bispecific antibodies at the concentration of 3 μg/mL on the SEB-activated human PBMC cells from two healthy donors. The various antibodies are as indicated and the stimulation of human PBMC cells are indicated by the secretion of IL-2. 21A: donor 1. 21B: donor 2

FIGS. 22A-22E include a set of graphs showings pharmacokinetics of exemplary anti-GITR/CD137 bispecific antibodies as indicated in mice. FIG. 22A: Clones Ly746. FIG. 22B: Clone Ly751. FIG. 22C: Clone Ly752. FIG. 22D: Clone Ly758. FIG. 22E: Clone Ly754.

FIG. 23 is the graph showing the anti-tumor activity of exemplary anti-GITR/CD137 antibodies in a human CD137 and human GITR knock-in mouse B16-OVA tumor model. Clones Ly754, TM685 and Ly1630 at doses as shown administered on day 8, 16, and 23 by intraperitoneal injection.

FIGS. 24A-24C include diagrams showing binding activity of exemplary anti-CD40/CD137 bi-specific antibodies. FIG. 24A: a chart showing CD40 binding activity of anti-CD40/CD137 bispecific antibodies as indicated to human CD40 expressed on CHO cells. Binding of these anti-CD40/CD137 bispecific antibodies are indicated by the mean fluorescence intensity (MFI). Clones Ly738, Ly739, Ly740, Ly741, Ly742, Ly743, Ly744, Ly745 and Ly253 at various concentrations as indicated. FIG. 24B: a chart showing CD137 binding activity of anti-CD40/CD137 bispecific antibodies as indicated to human CD137 expressed on CHO cells. Binding of these anti-CD40/CD137 bispecific antibodies are indicated by the mean fluorescence intensity (MFI). Clones Ly738, Ly739 and Ly1630 at various concentrations as indicated. FIG. 24C: a chart showing CD137 binding activity of anti-CD40/CD137 bispecific antibodies as indicated to human CD137 expressed on CHO cells. Binding of these anti-CD40/CD137 bispecific antibodies are indicated by the mean fluorescence intensity (MFI). Clones Ly740, Ly741, Ly742, Ly743, Ly744, Ly745 and Ly1630 at various concentrations as indicated.

FIGS. 25A-25B are charts showing OX40 binding activity of anti-OX40/CD137 bispecific antibodies as indicated to human OX40 expressed on CHO cells. The bars labeled “IgG control” served as controls. Binding of these anti-OX40/CD137 bispecific antibodies are indicated by the mean fluorescence intensity (MFI). 25A: Clones Ly762, Ly763, Ly764, Ly765, Ly766, Ly767 and Ly598 at various concentrations as indicated. 25B: Clones Ly762, Ly763, Ly764, Ly765, Ly766, Ly767, Ly768, Ly769 and Ly598 at various concentrations as indicated.

FIGS. 26A-26B are charts showing CD137 binding activity of anti-OX40/CD137 bispecific antibodies as indicated to human CD137 expressed on CHO cells. The bars labeled “IgG control” served as controls. Binding of these anti-OX40/CD137 bispecific antibodies are indicated by the mean fluorescence intensity (MFI). 26A: Clones Ly762, Ly763, Ly764, Ly765 and Ly1630 at various concentrations as indicated. 26B: Clones Ly766, Ly767, Ly768, Ly769 and Ly1630 at various concentrations as indicated.

FIG. 27 is a chart showing stimulation of human CD137 activation as indicated by IL8 secretion in a reporter assay by a number of anti-OX40/CD137 antibodies. The agonistic activity of these bispecific antibodies was evaluated in co-cultured with OX40 overexpressing CHO cells. The bars labeled as “IgG control” and “Mediun” served as controls. Clones Ly762, Ly763, Ly764, Ly765, Ly766, Ly767, Ly768, Ly769, Ly1630 and Ly598 at various concentrations are indicated for activating CD40 when cocultured with OX40 overexpressing CHO cells.

FIGS. 28A-28B are charts showing the stimulation activity of exemplary anti-OX40/CD137 bispecific antibodies on the SEB-activated human PBMC cells from two healthy donors. The various antibodies are as indicated and the stimulation of human PBMC cells are indicated by the secretion of IL-2. 28A: PBMC from donor 1 stimulated with Clones Ly762, Ly763, Ly764, Ly765, Ly766, Ly767, Ly768, Ly769, Ly598, Ly1630 alone or in combination with Ly598. 28B: PBMC from donor 2 stimulated with Clones Ly762, Ly763, Ly764, Ly765, Ly766, Ly767, Ly768, Ly769, Ly598, Ly1630 alone or in combination with Ly598.

FIGS. 29A-29E include a set of graphs showings pharmacokinetics of anti-OX40/CD137 bispecific antibodies as indicated in mice. FIG. 29A: Clones Ly763. FIG. 29B: Clone Ly765. FIG. 29C: Clone Ly766. FIG. 29D: Clone Ly767. FIG. 29E: Clone Ly768.

FIGS. 30A-30B include graphs showing anti-tumor activity of exemplary anti-OX40/CD137 antibodies in a mouse model transplanted with human PBMCs and human melanoma tumor cells. FIG. 30A: Bi-specific clone Ly763 and parent clones Ly598 and Ly1630 were administered to the mice on day 0 and day 15 by intraperitoneal injection at the indicated doses. FIG. 30B: Clones Ly763, Ly765, Ly766, and Ly768 in combination with Keytruda® were administered to the mice on day 0 and day 14 at the indicated doses.

### DETAILED DESCRIPTION OF THE INVENTION

Provided herein are antibodies specific to antibodies specific to GITR (i.e., anti-GITR antibodies). Also provided herein are bi-specific antibodies comprising a first antibody moiety specific to CD137 and a second antigen which may be, but not limited to, an immune modulator. Examples include, but are not limited to, PD-1, PD-L1, GITR, CD40 or OX40. Such antibodies or bi-specific antibodies may be used for various therapeutic, diagnostic, or research purposes. For example, the antibodies may be used in modulating immune responses such as anti-tumor immune responses in subjects in need of such treatment. The antibodies may also be used for cancer treatment or cancer diagnosis.

### I. Bi-Specific Antibodies Comprising Anti-CD137 Binding Molecules

In some aspects, the present disclosure also provides bi-specific antibodies each comprising at least two antibody moieties, one specific to CD137 and the other one specific to another antigen of interest, for example, an immune checkpoint or modulator molecule. Examples of the other antigen specific to the bi-specific antibodies disclosed herein include, but are not limited to, PD-1, PD-L1, GITR, CD40, or OX40.

Each antibody portion in the bispecific antibody as described herein can be an antibody in any form, including, but not limited to, intact (i.e., full-length) antibodies, antigen-binding fragments thereof (such as Fab, Fab′, F(ab′).sub.2, Fv), single chain antibodies (scFv antibodies), and tetravalent antibodies. In some embodiments, the bispecific antibody is tetravalent, which comprises two binding sites for CD137 and two binding sites for the other antigen (e.g., PD-1, PD-L1, GITR, CD40, or OX40).

In some embodiments, the antibody moieties in any of the bi-specific antibodies described herein specifically bind to the corresponding target antigen(s) (e.g., CD137, PD-1, PD-L1, GITR, CD40, or OX40) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., those listed above) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method). Alternatively, or in addition, the antibodies described herein may specifically binds the human antigen or a fragment thereof as relative to the monkey counterpart, or vice versa (e.g., having a binding affinity at least 10-fold higher to one antigen than the other as determined in the same assay under the same assay conditions). In other instances, the antibodies described herein may cross-react to human and a non-human antigen (e.g., monkey), e.g., the difference in binding affinity to the human and the non-human antigen is less than 5-fold, e.g., less than 2-fold, or substantially similar.

In some embodiments, an antibody moiety in any of the bi-specific antibodies as described herein has a suitable binding affinity for the target antigen(s) (e.g., CD137, PD-1, PD-L1, GITR, CD40, or OX40) or antigenic epitopes thereof. As used herein, “binding affinity” refers to the apparent association constant or K_{A}. The K_{A} is the reciprocal of the dissociation constant (K_{D}). The antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Any of the bi-specific antibodies disclosed herein may be in any bi-specific antibody format known in the art, for example, BsIgG, BsAb fragment, Bispecific fusion proteins, or BsAb conjugate. See, e.g., Mol. Immunol. 67(2):95-106 (2015).

In some embodiments, a first antibody moiety binding to a first antigen in the bi-specific antibody, e.g., the antibody moiety that binds CD137, can be in a single-chain fragment (scFv) format, and a second antibody moiety binding to a second antigen is in a multi-chain antibody format that comprises a heavy chain comprising a V_{H} and a heavy chain constant region or a portion thereof, and a light chain comprising a V_{L} and a light chain constant region (e.g., a kappa chain). Alternatively, the antibody moiety that binds CD137 may be in the multi-chain antibody format as disclosed herein and the antibody moiety that binds the other antigen can be in an scFv format. Any scFv fragment in a bi-specific antibody may be in V_{H}→V_{L} orientation. Alternatively, it can be in the V_{L}→V_{H} orientation.

In some examples, the bi-specific antibody may comprise two chains: a first chain being a fusion protein of the scFv fragment of one antibody moiety and the heavy chain or the light chain of the other antibody moiety, and the second chain being the other chain of the other antibody moiety. For example, the bi-specific antibody may comprise a first chain that is a fusion protein of a scFv fragment of a first antibody moiety binding to a first antigen (e.g., CD137) fused to the heavy chain of a second antibody moiety, which binds to a second antigen (e.g., PD-1, PD-L1, GITR, CD40, or OX40), and a second chain which is the light chain of the second antibody moiety. In other examples, the bi-specific antibody may comprise a first chain that is a fusion protein of a scFv fragment of a first antibody moiety binding to a first antigen (e.g., CD137) fused to the light chain of a second antibody moiety, which binds to a second antigen (e.g., PD-1, PD-L1, GITR, CD40, or OX40), and a second chain, which is the heavy chain of the second antibody moiety. In any of the fusion chains, the scFv fragment and the heavy or light chain may be in any order. In some instances, the scFv can be located at the N-terminus. In other instances, the heavy or light chain may be located at the N-terminus.

In some examples, the bi-specific antibody may comprise two chains: (i) a first polypeptide comprising the V_{L} fragment of a first antibody moiety and a heavy chain comprising the V_{H} fragment of a second antibody moiety and an Fc fragment (e.g., a whole Fc fragment or a portion thereof such as CH2-CH3); and (ii) a second polypeptide comprising the V_{H} fragment of the first antibody moiety and the V_{L} fragment of the second antibody moiety. In the first polypeptide, the V_{L} fragment may be located at the N-terminus and the heavy chain may be located at the C-terminus. Alternatively, the V_{L} fragment may be located at the C-terminus and the heavy chain may be located at the N-terminus of the first polypeptide. Similarly, the second polypeptide may have the V_{H} fragment at the N-terminus and the V_{L} fragment at the C-terminus. Alternatively, the second polypeptide may have the V_{H} fragment at the C-terminus and the V_{L} fragment at the N-terminus.

For example, the bi-specific antibody may comprise: (i) a first polypeptide comprising the V_{L} fragment of a first antibody moiety that binds CD137 and a heavy chain comprising the V_{H} fragment of a second antibody that binds PD-1, PD-L1, GITR, CD40, or OX40 and an Fc fragment; and (ii) a second polypeptide comprising the V_{H} fragment of the first antibody moiety and the V_{L} fragment of the second antibody moiety. Alternatively, the bi-specific antibody may comprise (i) a first polypeptide comprising the V_{L} fragment of a first antibody moiety that binds PD-1, PD-L1, GITR, CD40, or OX40 and a heavy chain comprising the V_{H} fragment of a second antibody that binds CD137 and an Fc fragment; and (ii) a second polypeptide comprising the V_{H} fragment of the first antibody moiety and the V_{L} fragment of the second antibody moiety.

In other examples, the bi-specific antibody may comprise two chains: (i) a first polypeptide comprising the V_{H} fragment of a first antibody moiety and a heavy chain of a second antibody moiety (comprising the V_{H} fragment and an Fc fragment), and (ii) a second polypeptide comprising the V_{L} fragment of the first antibody moiety and the light chain of the second antibody moiety (e.g., comprising a light chain variable region and a light chain constant region). In the first polypeptide, the V_{H} fragment of the first antibody moiety may be located at the N-terminus. Alternatively, it may be located at the C-terminus. In the second polypeptide, the V_{L} fragment of the first antibody moiety may be located at the N-terminus. Alternatively, it may be located at the C-terminus. In some instances, the first antibody moiety binds CD137 and the second antibody moiety binds PD-1, PD-L1, GITR, CD40, or OX40. In other instances, the first antibody moiety binds PD-1, PD-L1, GITR, CD40, or OX40 and the second antibody moiety binds CD137.

In some embodiments, a bi-specific antibody as disclosed herein are in a three-chain format, comprising a first polypeptide, a second polypeptide, and a third polypeptide. The first polypeptide comprises the heavy chain of the first antibody moiety (e.g., binding to CD137) in the bi-specific antibody fused to the light chain of the second antibody moiety (e.g., binding to the second antigen such as PD-1, PD-L1, GITR, CD40, or OX40). The second and third polypeptides comprise the light chain of the first antibody moiety and the heavy chain of the second antibody moiety, respectively. In some instances, the heavy chain of the second antibody moiety may comprise a V_{H} fragment and a heavy chain constant region such as CH1. Alternatively, the first polypeptide comprises the heavy chain of the second antibody moiety (e.g., binding to the second antigen such as PD-1, PD-L1, GITR, CD40, or OX40) fused to the light chain of the first antibody moiety (e.g., binding to CD137). The second and third polypeptides comprise the light chain of the second antibody moiety and the heavy chain of the first antibody moiety, respectively. In some instances, the heavy chain of the first antibody moiety may comprise a V_{H} fragment and a heavy chain constant region such as CH1. In some instances, the light chain fragment in the first polypeptide can be located at the N-terminus. Alternatively, it may be located at the C-terminus.

A peptide linker may be located between two fragments in a bi-specific antibody disclosed herein, for example, between the V_{H} and V_{L} portions in a scFv fragment, between the scFv fragment and the heavy or light chain in a fusion chain, or between the heavy chain and light chain in a fusion polypeptide. Exemplary peptide linker includes the linker of (GGGGS)ₙ (SEQ ID NOs:128-133), in which n can be an integer between 1-6, for example, 1, 2, 3, 4, 5, or 6. Any of the peptide linkers described herein, e.g., the SGGGS (SEQ ID NO:134) linker or the (GGGGS)₄ (SEQ ID NO:135) linker, can comprise naturally occurring amino acids and/or non-naturally occurring amino acids. Naturally occurring amino acids include alanine (Ala), arginine (Arg), asparagine (Asn), aspartic acid (Asp), cysteine (Cys), glutamic acid (Glu), glutamine (Gin), glycine (Gly), histidine (His), isoleucine (He), leucine (Leu), lysine (Lys) methionine (Met), ornithine (Orn), phenylalanine (Phe), proline (Pro), serine (Ser), threonine (Thr), tryptophan (Trp), tyrosine (Tyr), and valine (Val). Non-naturally occurring amino acids can include protected amino acids such as naturally occurring amino acids protected with groups such as acetyl, formyl, tosyl, nitro and the like. Non-limiting examples of non-naturally occurring amino acids include azidohomoalanine, homopropargylglycine, homoallylglycine, p-bromophenylalanine, p-iodophenylalanine, azidophenylalanine, acetylphenylalanine or ethynylephenylalanine, amino acids containing an internal alkene such as trans-crotylalkene, serine allyl ether, allyl glycine, propargyl glycine, vinyl glycine, pyrrolysine, N-sigma-o-azidobenzyloxycarbonyl-L-Lysine (AzZLys), N-sigma-propargyloxycarbonyl-L-Lysine, N-sigma-2-azidoethoxycarbonyl-L-Lysine, N-sigma-tert-butyloxycarbonyl-L-Lysine (BocLys), N-sigma-allyloxycarbonyl-L-Lysine (AlocLys), N-sigma-acetyl-L-Lysine (AcLys), N-sigma-benzyloxycarbonyl-L-Lysine (ZLys), N-sigma-cyclopentyloxycarbonyl-L-Lysine (CycLys), N-sigma-D-prolyl-L-Lysine, N-sigma-nicotinoyl-L-Lysine (NicLys), N-sigma-N-Me-anthraniloyl-L-Lysine (NmaLys), N-sigma-biotinyl-L-Lysine, N-sigma-9-fluorenylmethoxycarbonyl-L-Lysine, N-sigma-methyl-L-Lysine, N-sigma-dimethyl-L-Lysine, N-sigma-trimethyl-L-Lysine, N-sigma-isopropyl-L-Lysine, N-sigma-dansyl-L-Lysine, N-sigma-o,p-dinitrophenyl-L-Lysine, N-sigma-p-toluenesulfonyl-L-Lysine, N-sigma-DL-2-amino-2carboxyethyl-L-Lysine, N-sigma-phenylpyruvamide-L-Lysine, N-sigma-pyruvamide-L-Lysine, azidohomoalanine, homopropargylglycine, homoallylglycine, p-bromophenylalanine, p-iodophenylalanine, azidophenylalanine, acetylphenylalanine or ethynylephenylalanine, amino acids containing and an internal alkene such as trans-crotylalkene, serine allyl ether, allyl glycine, propargyl glycine, and vinyl glycine.

Anti-CD137 Portion

Any antibody capable of binding to CD137 can be used in constructing the bi-specific antibodies disclosed herein. In some examples, the anti-CD137 portion of the bi-specific antibody may be derived from any of the anti-CD137 antibodies disclosed herein (e.g., Ly1630 or derivatives thereof as disclosed herein; see, e.g., Example 1).

As used herein, an antibody moiety in a bi-specific antibody “derived from” a parent antibody means that the parent antibody is used as a starting material for making the bi-specific antibody as known in the art. The antibody moiety may comprise the same heavy chain and/or light chain CDRs as those of the parent antibody. Two antibodies having the same V_{H} and/or V_{L} CDRs means that their CDRs are identical when determined by the same approach (e.g., the Kabat definition, the Chothia definition, the AbM definition, and/or the contact definition as known in the art).

Alternatively, the antibody moiety may comprise substantially similar heavy chain and/or light chain CDRs as those of the parent antibody (e.g., comprising no more than 5, 4, 3, 2, or 1 amino acid residue variations as compared with the parent antibody). In some instances, the antibody moiety in the bi-specific antibody may have the same heavy chain variable region and/or the same light chain variable region as the parent antibody. For example, the antibody moiety in the bi-specific antibody may have the same heavy chain and/or the same light chain as the parent antibody.

In specific examples, Ly1630 or humanized antibodies derived there from may be used as a starting material for making any of the bi-specific antibodies disclosed herein.

Second Antibody Portion in Bi-Specific Antibodies

In addition to the first antibody moiety binding to CD137, the bi-specific antibodies disclosed herein comprise a second antibody moiety capable of binding to a suitable antigen, such as a tumor antigen or an immune checkpoint molecule (e.g., those that negatively or positively regulates immune responses). Examples include PD-1, PD-L1, GITR, CD40, or OX40.

Anti-CD137/PD-1 Bi-Specific Antibodies

In some embodiments, the second antibody moiety in the bi-specific antibodies disclosed herein binds PD-1, for example, human PD-1. Any antibody capable of binding to PD-1 can be used in constructing the bi-specific antibodies disclosed herein. In some examples, the anti-PD-1 portion of the bi-specific antibody described herein may be derived from any of the anti-PD-1 antibodies provided herein (e.g., Ly516). The anti-PD-1 antibody moiety may comprise the same heavy chain and/or light chain CDRs as a parent antibody, e.g., Ly516. Alternatively, the antibody moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-PD-1 antibody moiety in the bi-specific antibody may have the same heavy chain variable region and/or the same light chain variable region as the parent antibody. For example, the antibody moiety in the bi-specific antibody may have the same heavy chain and/or the same light chain as the parent antibody.

In some examples, the anti-CD137/PD-1 bi-specific antibodies may comprise an anti-CD137 moiety in scFv format and an anti-PD-1 moiety in multi-chain format. The anti-CD137 scFv fragment may be derived from any of the anti-CD137 antibodies disclosed herein, for example, Ly1630. For example, the bi-specific antibody may comprise a first chain comprising the scFv fragment fused with the heavy chain of the anti-PD-1 antibody such as that of Ly516, and a second chain that is the light chain of the anti-PD-1 antibody.

Alternatively, the bi-specific antibody may comprise a first chain comprising the scFv fragment may be fused with the heavy chain of the anti-PD-1 antibody such as that of Ly516, and a second chain that is the heavy chain of the anti-PD-1 antibody. In some instances, the heavy chain of the anti-PD-1 antibody may comprise a mutated Fc region having altered binding affinity and/or binding specificity to an Fc receptor such as those described herein.

In some examples, the anti-CD137/PD-1 bi-specific antibodies may comprise an anti-PD-1 moiety in scFv format and an anti-CD137 moiety in multi-chain format. The anti-PD-1 scFv fragment may be derived from any of the anti-PD-1 antibodies disclosed herein, for example, Ly516. For example, the bi-specific antibody may comprise a first chain comprising the scFv fragment fused with the heavy chain of the anti-CD137 antibody such as that of Ly1630, and a second chain that is the light chain of the anti-CD137 antibody. Alternatively, the bi-specific antibody may comprise a first chain comprising the scFv fragment may be fused with the heavy chain of the anti-CD137 antibody such as that of Ly1630, and a second chain that is the heavy chain of the anti-CD137 antibody. In some instances, the heavy chain of the anti-CD137 antibody may comprise a mutated Fc region having altered binding affinity and/or binding specificity to an Fc receptor such as those described herein.

In some embodiments, the anti-CD137/PD-1 bi-specific antibody disclosed herein may be in a three-chain format as disclosed herein. Such a bi-specific antibody may comprise a first polypeptide comprises the heavy chain of the first antibody moiety (e.g., binding to CD137) fused to the light chain of second antibody moiety (e.g., binding to PD-1), a second polypeptide comprising the light chain of the first antibody moiety, and a third polypeptide comprising the heavy chain of the second antibody moiety. In some instances, the heavy chain of the second antibody moiety may comprise a V_{H} fragment and a heavy chain constant region such as CH1. Alternatively, the bi-specific antibody may comprise a first polypeptide comprising the heavy chain of the second antibody moiety (e.g., binding to PD-1) fused to the light chain of the first antibody moiety (e.g., binding to CD137), a second polypeptide comprising the light chain of the second antibody moiety, and a third polypeptide comprising the heavy chain of the first antibody moiety. In some instances, the heavy chain of the first antibody moiety may comprise a V_{H} fragment and a heavy chain constant region such as CH1. In some instances, the light chain fragment in the first polypeptide can be located at the N-terminus. Alternatively, it can be located at the C-terminus.

In some examples, the bi-specific antibody may comprise two chains: (i) a first polypeptide comprising the V_{H} fragment of the first antibody moiety and the heavy chain of the second antibody moiety, and (ii) a second chain comprising the V_{L} fragment of the first antibody moiety and the light chain of the second antibody moiety. In some instances, the first antibody moiety binds CD137 and the second antibody moiety binds PD-1. In other instances, the first antibody moiety binds PD-1 and the second antibody moiety binds CD137

In other examples, the bi-specific antibody may comprise two chains: (i) a first polypeptide comprising the V_{L} fragment of a first antibody moiety and a heavy chain comprising the V_{H} fragment of a second antibody moiety and an Fc fragment (e.g., a whole Fc fragment or a portion thereof such as CH2-CH3); and (ii) a second polypeptide comprising the V_{H} fragment of the first antibody moiety and the V_{L} fragment of the second antibody moiety. In some instances, the first antibody moiety binds CD137 and the second antibody moiety binds PD-1. In other instances, the first antibody moiety binds PD-1 and the second antibody moiety binds CD137

Exemplary anti-CD137/PD-1 bi-specific antibodies are provided in Example 1, which are within the scope of the present disclosure.

Anti-CD137/PD-L1 Bi-Specific Antibodies

In some embodiments, the second antibody moiety in the bi-specific antibodies disclosed herein binds PD-L1, for example, human PD-L1. Any antibody capable of binding to PD-L1 can be used in constructing the bi-specific antibodies disclosed herein. In some examples, the anti-PD-L1 portion of the bi-specific antibody described herein may be derived from any of the anti-PD-L1 antibodies provided herein (e.g., Ly076). The anti-PD-L1 antibody moiety may comprise the same heavy chain and/or light chain CDRs as a parent antibody, e.g., Ly076. Alternatively, the antibody moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-PD-L1 antibody moiety in the bi-specific antibody may have the same heavy chain variable region and/or the same light chain variable region as the parent antibody. For example, the antibody moiety in the bi-specific antibody may have the same heavy chain and/or the same light chain as the parent antibody.

In some examples, the anti-CD137/PD-L1 bi-specific antibodies may comprise an anti-CD137 moiety in scFv format and an anti-PD-L1 moiety in multi-chain format. The anti-CD137 scFv fragment may be derived from any of the anti-CD137 antibodies disclosed herein, for example, Ly1630. For example, the bi-specific antibody may comprise a first chain comprising the scFv fragment fused with the heavy chain of the anti-PD-L1 antibody such as that of Ly076, and a second chain that is the light chain of the anti-PD-L1 antibody. Alternatively, the bi-specific antibody may comprise a first chain comprising the scFv fragment may be fused with the heavy chain of the anti-PD-L1 antibody such as that of Ly076, and a second chain that is the heavy chain of the anti-PD-1 antibody. In some instances, the heavy chain of the anti-PD-L1 antibody may comprise a mutated Fc region having altered binding affinity and/or binding specificity to an Fc receptor such as those described herein.

In some examples, the anti-CD137/PD-L1 bi-specific antibodies may comprise an anti-PD-L1 moiety in scFv format and an anti-CD137 moiety in multi-chain format. The anti-PD-L1 scFv fragment may be derived from any of the anti-PD-L1 antibodies disclosed herein, for example, Ly076. For example, the bi-specific antibody may comprise a first chain comprising the scFv fragment fused with the heavy chain of the anti-CD137 antibody such as that of Ly1630, and a second chain that is the light chain of the anti-CD137 antibody. Alternatively, the bi-specific antibody may comprise a first chain comprising the scFv fragment may be fused with the heavy chain of the anti-CD137 antibody such as that of Ly1630, and a second chain that is the heavy chain of the anti-CD137 antibody. In some instances, the heavy chain of the anti-CD137 antibody may comprise a mutated Fc region having altered binding affinity and/or binding specificity to an Fc receptor such as those described herein.

In some embodiments, the anti-CD137/PD-L1 bi-specific antibody may be in the three-chain format as disclosed herein.

Exemplary anti-CD137/PD-L1 bi-specific antibodies are provided in Example 2, which are within the scope of the present disclosure.

Anti-CD137/GITR Bi-Specific Antibodies

In some embodiments, the second antibody moiety in the bi-specific antibodies disclosed herein binds GITR, for example, human GITR. Any antibody capable of binding to GITR can be used in constructing the bi-specific antibodies disclosed herein. In some examples, the anti-GITR portion of the bi-specific antibody described herein may be derived from any of the anti-GITR antibodies provided herein (e.g., TM676, TM677 or TM685). The anti-GITR antibody moiety may comprise the same heavy chain and/or light chain CDRs as a parent antibody, e.g., TM676, TM677 or TM685. Alternatively, the antibody moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-GITR antibody moiety in the bi-specific antibody may have the same heavy chain variable region and/or the same light chain variable region as the parent antibody. For example, the antibody moiety in the bi-specific antibody may have the same heavy chain and/or the same light chain as the parent antibody.

In some examples, the anti-CD137/GITR bi-specific antibodies may comprise an anti-CD137 moiety in scFv format and an anti-GITR moiety in multi-chain format. The anti-CD137 scFv fragment may be derived from any of the anti-CD137 antibodies disclosed herein, for example, Ly1630. For example, the bi-specific antibody may comprise a first chain comprising the scFv fragment fused with the heavy chain of the anti-GITR antibody such as that of TM676, TM677 or TM685, and a second chain that is the light chain of the anti-GITR antibody. Alternatively, the bi-specific antibody may comprise a first chain comprising the scFv fragment may be fused with the heavy chain of the anti-GITR antibody such as that of TM676, TM677 or TM685, and a second chain that is the heavy chain of the anti-GITR antibody. In some instances, the heavy chain of the anti-GITR antibody may comprise a mutated Fc region having altered binding affinity and/or binding specificity to an Fc receptor such as those described herein.

In some examples, the anti-GITR/CD137 bi-specific antibodies may comprise an anti-GITR moiety in scFv format and an anti-CD137 moiety in multi-chain format. The anti-GITR scFv fragment may be derived from any of the anti-GITR antibodies disclosed herein, for example, TM676, TM677 or TM685. For example, the bi-specific antibody may comprise a first chain comprising the scFv fragment fused with the heavy chain of the anti-CD137 antibody such as that of Ly1630, and a second chain that is the light chain of the anti-CD137 antibody. Alternatively, the bi-specific antibody may comprise a first chain comprising the scFv fragment may be fused with the heavy chain of the anti-CD137 antibody such as that of Ly1630, and a second chain that is the heavy chain of the anti-CD137 antibody. In some instances, the heavy chain of the anti-CD137 antibody may comprise a mutated Fc region having altered binding affinity and/or binding specificity to an Fc receptor such as those described herein.

In some examples, the anti-GITR/CD137 bi-specific antibody disclosed herein may be in the three-chain or any of the two-chain formats as disclosed herein.

Exemplary anti-CD137/GITR bi-specific antibodies are provided in Example 3, which are within the scope of the present disclosure.

Anti-CD137/CD40 Bi-Specific Antibodies

In some embodiments, the second antibody moiety in the bi-specific antibodies disclosed herein binds CD40, for example, human CD40. Any antibody capable of binding to CD40 can be used in constructing the bi-specific antibodies disclosed herein. In some examples, the anti-CD40 portion of the bi-specific antibody described herein may be derived from any of the anti-CD40 antibodies provided herein (e.g., Ly253). The anti-CD40 antibody moiety may comprise the same heavy chain and/or light chain CDRs as a parent antibody, e.g., Ly253. Alternatively, the antibody moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-CD40 antibody moiety in the bi-specific antibody may have the same heavy chain variable region and/or the same light chain variable region as the parent antibody. For example, the antibody moiety in the bi-specific antibody may have the same heavy chain and/or the same light chain as the parent antibody.

In some examples, the anti-CD137/CD40 bi-specific antibodies may comprise an anti-CD137 moiety in scFv format and an anti-CD40 moiety in multi-chain format. The anti-CD137 scFv fragment may be derived from any of the anti-CD137 antibodies disclosed herein, for example, Ly1630. For example, the bi-specific antibody may comprise a first chain comprising the scFv fragment fused with the heavy chain of the anti-CD40 antibody such as that of Ly253, and a second chain that is the light chain of the anti-CD40 antibody. Alternatively, the bi-specific antibody may comprise a first chain comprising the scFv fragment may be fused with the heavy chain of the anti-CD40 antibody such as that of Ly253, and a second chain that is the heavy chain of the anti-CD40 antibody. In some instances, the heavy chain of the anti-CD40 antibody may comprise a mutated Fc region having altered binding affinity and/or binding specificity to an Fc receptor such as those described herein.

In some examples, the anti-CD137/CD40 bi-specific antibodies may comprise an anti-CD40 moiety in scFv format and an anti-CD137 moiety in multi-chain format. The anti-CD40 scFv fragment may be derived from any of the anti-CD40 antibodies disclosed herein, for example, Ly253. For example, the bi-specific antibody may comprise a first chain comprising the scFv fragment fused with the heavy chain of the anti-CD137 antibody such as that of Ly1630, and a second chain that is the light chain of the anti-CD137 antibody. Alternatively, the bi-specific antibody may comprise a first chain comprising the scFv fragment may be fused with the heavy chain of the anti-CD137 antibody such as that of Ly1630, and a second chain that is the heavy chain of the anti-CD137 antibody. In some instances, the heavy chain of the anti-CD137 antibody may comprise a mutated Fc region having altered binding affinity and/or binding specificity to an Fc receptor such as those described herein.

In some examples, the anti-CD137/CD40 bi-specific antibody disclosed herein may be in the three-chain or any of the two-chain formats as disclosed herein.

Exemplary anti-CD137/CD40 bi-specific antibodies are provided in Example 4, which are within the scope of the present disclosure.

Anti-CD137/OX40 Bi-Specific Antibodies

In some embodiments, the second antibody moiety in the bi-specific antibodies disclosed herein binds OX40, for example, human OX40. Any antibody capable of binding to OX40 can be used in constructing the bi-specific antibodies disclosed herein. In some examples, the anti-OX40 portion of the bi-specific antibody described herein may be derived from any of the anti-OX40 antibodies provided herein (e.g., Ly598). The anti-OX40 antibody moiety may comprise the same heavy chain and/or light chain CDRs as a parent antibody, e.g., Ly598. Alternatively, the antibody moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-OX40 antibody moiety in the bi-specific antibody may have the same heavy chain variable region and/or the same light chain variable region as the parent antibody. For example, the antibody moiety in the bi-specific antibody may have the same heavy chain and/or the same light chain as the parent antibody.

In some examples, the anti-CD137/OX40 bi-specific antibodies may comprise an anti-CD137 moiety in scFv format and an anti-OX40 moiety in multi-chain format. The anti-CD137 scFv fragment may be derived from any of the anti-CD137 antibodies disclosed herein, for example, Ly1630. For example, the bi-specific antibody may comprise a first chain comprising the scFv fragment fused with the heavy chain of the anti-OX40 antibody such as that of Ly598, and a second chain that is the light chain of the anti-OX40 antibody. Alternatively, the bi-specific antibody may comprise a first chain comprising the scFv fragment may be fused with the heavy chain of the anti-OX40 antibody such as that of Ly598, and a second chain that is the heavy chain of the anti-OX40 antibody. In some instances, the heavy chain of the anti-OX40 antibody may comprise a mutated Fc region having altered binding affinity and/or binding specificity to an Fc receptor such as those described herein.

In some examples, the anti-CD137/OX40 bi-specific antibodies may comprise an anti-OX40 moiety in scFv format and an anti-CD137 moiety in multi-chain format. The anti-OX40 scFv fragment may be derived from any of the anti-OX40 antibodies disclosed herein, for example, Ly598. For example, the bi-specific antibody may comprise a first chain comprising the scFv fragment fused with the heavy chain of the anti-CD137 antibody such as that of Ly1630, and a second chain that is the light chain of the anti-CD137 antibody. Alternatively, the bi-specific antibody may comprise a first chain comprising the scFv fragment may be fused with the heavy chain of the anti-CD137 antibody such as that of Ly1630, and a second chain that is the heavy chain of the anti-CD137 antibody. In some instances, the heavy chain of the anti-CD137 antibody may comprise a mutated Fc region having altered binding affinity and/or binding specificity to an Fc receptor such as those described herein.

In some examples, the anti-CD137/OX40 bi-specific antibody may be in the three-chain or any of the two-chain formats as disclosed herein.

Exemplary anti-CD137/OX40 bi-specific antibodies are provided in Example 5, which are within the scope of the present disclosure.

In any of the bi-specific antibodies disclosed herein, a heavy chain of the first antibody moiety, the second antibody moiety, or both, if applicable, may contain a mutated Fc region as compared with a wild-type counterpart such that the antibody has an altered binding affinity and/or binding specificity to an Fc receptor. In some examples, the antibody heavy chain may comprise a modified Fc region having an elevated binding affinity to FcγRIIB (CD32B), which may engage FcγRIIB-expressing cells efficiently, or a modified Fc region having low or no binding to all Fcγ receptors, thereby enhancing therapeutic effects. Examples of mutated Fc regions are provided herein or disclosed in WO/2018/183520 and PCT/US2019/053505 (filed on Sep. 27, 2019), the relevant disclosures of each of which are incorporated by reference for the purpose and subject matter referenced herein. Alternatively, the antibodies described herein may comprise a modified constant region. For example, it may comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

### II. Anti-GITR Antibodies

In some aspects, the present disclosure provides antibodies specific to a glucocorticoid induced TNFR-related (GITR) polypeptide (“anti-GITR antibodies), which may be of any source, for example, human and/or monkey GITR. Such anti-GITR antibodies may specifically bind GITR of a particular species (e.g., human GITR). Alternatively, the anti-GITR antibodies described herein may cross-react with GITR antigens of different species (e.g., binding to both human and monkey GITR). In some instances, the anti-GITR antibodies described herein can bind cell surface GITR, for example, GITR expressed on cells (e.g., immune cells) that naturally express GITR on the surface.

GITR, also known as TNF receptor superfamily member 18 (TNFRSF18) or CD357, is an immune costimulatory receptor molecule of the tumor necrosis factor (TNF) superfamily. Director agonistic effects arising from of anti-GITR therapy may lead to antitumor effects. GITR is a protein well known in the art. For example, the structural information of human GITR can be find under Gene ID: 8784.

As used herein, an antibody (interchangeably used in plural form) refers to an immunoglobulin molecule capable of specific binding to a target, e.g., any of the target antigens disclosed herein, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

The anti-GITR antibodies described herein can be murine, rat, human, or any other origin (including chimeric or humanized antibodies). Such antibodies are non-naturally occurring, i.e., would not be produced in an animal without human act (e.g., immunizing such an animal with a desired antigen or fragment thereof or isolated from antibody libraries).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-GITR antibodies described herein may bind to the same epitope of a reference anti-GITR antibody or competes against the reference antibody from binding to the GITR antigen. In some instances, the reference anti-GITR antibody is Lyv392 or Lyv396. The structural information of these two reference antibodies are provided in Example 3 below. An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as a reference antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the reference antibody. Whether two antibodies compete against each other from binding to the cognate antigen can be determined by a competition assay, which is well known in the art.

In some embodiments, the anti-GITR antibody as described herein comprises a heavy chain variable region that comprises a heavy chain CDR1 region (HC CDR1), a heavy chain CDR2 region (HC CDR2), and a heavy chain CDR3 region (HC CDR3) connected by heavy chain framework regions. Alternatively or in addition, the anti-GITR may comprise a light chain variable region that comprises a light chain CDR1 region (LC CDR1), a light chain CDR2 region (LC CDR2), and a light chain CDR3 region (LC CDR3) connected by light chain framework regions. In some examples, the anti-GITR antibody disclosed herein may comprise the same heavy chain CDRs and/or the same light chain CDRs as reference antibody Lyv392 (see details in Example 3 below). In other examples, the anti-GITR antibody disclosed herein may comprise the same heavy chain CDRs and/or the same light chain CDRs as reference antibody Lyv396 (see details in Example 3 below).

Also within the scope of the present disclosure are functional variants of reference antibody Lyv392 or Lyv396. Such functional variants are substantially similar to the reference antibody, both structurally and functionally. A functional variant comprises substantially the same V_{H} and V_{L} CDRs as the reference antibody. For example, it may comprise only up to 5 (e.g., 4, 3, 2, or 1) amino acid residue variations in the total heavy chain CDR regions of the reference antibody and/or comprise only up to 5 (e.g., 4, 3, 2, or 1) amino acid residue variations in the total light chain CDR regions of the reference antibody. In some examples, the functional variant may comprise up to 8 (e.g., 7, 6, 5, 4, 3, 2, or 1) amino acid residue variations in the total heavy and light chain CDRs relative to those of the reference antibody. Such functional variants may bind the same epitope of GITR with substantially similar affinity (e.g., having a K_{D} value in the same order). Alternatively or in addition, the amino acid residue variations are conservative amino acid residue substitutions as disclosed herein.

In some embodiments, the anti-GITR antibody may comprise heavy chain CDRs that are at least 80% (e.g., 85%, 90%, 95%, or 98%) sequence identity, individually or collectively, as compared with the V_{H} CDRs of Lyv392 described herein. Alternatively or in addition, the anti-GITR antibody may comprise light chain CDRs that are at least 80% (e.g., 85%, 90%, 95%, or 98%) sequence identity, individually or collectively, as compared with the V_{L} CDRs as Lyv392.

In other embodiments, the anti-GITR antibody may comprise heavy chain CDRs that are at least 80% (e.g., 85%, 90%, 95%, or 98%) sequence identity, individually or collectively, as compared with the V_{H} CDRs of Lyv396 described herein. Alternatively or in addition, the anti-GITR antibody may comprise light chain CDRs that are at least 80% (e.g., 85%, 90%, 95%, or 98%) sequence identity, individually or collectively, as compared with the V_{L} CDRs as Lyv396.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Humanized Anti-GITR Antibodies

In some embodiments, the anti-GITR antibodies disclosed herein are humanized antibodies derived from a non-human parent antibody clone, for example, a murine antibody binding to GITR such as human GITR. Humanized antibodies refer to forms of non-human (e.g., murine) antibodies that are specific chimeric immunoglobulins, immunoglobulin chains, or antigen-binding fragments thereof that contain minimal sequence derived from the non-human immunoglobulin parent. For the most part, humanized antibodies are human immunoglobulins (recipient antibody), in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody. This is also termed one or more CDRs “derived from” one or more CDRs from the original antibody. Humanized antibodies may also involve affinity maturation.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions can be used to substitute for the corresponding residues in the human acceptor genes.

In some embodiments, the anti-GITR antibodies disclosed herein are humanized antibodies derived from murine parent clone Lyv392, which are disclosed in Example 3 below. Such a humanized antibody may comprise a heavy chain framework of IGHV4-59*01 and/or a light chain framework of IGKV3-11*01. In addition, such a humanized antibody may comprise the same heavy chain and/or light chain complementary determining regions (CDRs) as the murine parent clone. Alternatively, the humanized anti-GITR antibodies, which may comprise the heavy chain framework of IGHV4-59*01 and/or a light chain framework of IGKV3-11*01, may comprise one or more amino acid residue variations in one or more CDR regions as relative to the corresponding CDR regions of the murine parent Lyv392. For example, the humanized antibody may comprise up to 5 (e.g., up to 4, 3, 2, or 1) amino acid residues in the three heavy chain CDRs collectively. In other examples, the humanized antibody may comprise up to 5 (e.g., up to 4, 3, 2, or 1) amino acid residues in the three light chain CDRs collectively. In yet other examples, the humanized antibody may comprise up to 8 (e.g., up to 7, 6, 5, 4, 3, 2, or 1) amino acid residues in the three heavy chain CDRs and the three light chain CDRs collectively.

In some embodiments, the anti-GITR antibodies disclosed herein are humanized antibodies derived from murine parent clone Lyv396, which are disclosed in Example 3 below. Such a humanized antibody may comprise a heavy chain framework of IGHV4-59*01 and/or a light chain framework of IGKV3-11*01. In addition, such a humanized antibody may comprise the same heavy chain and/or light chain complementary determining regions (CDRs) as the murine parent clone. Alternatively, the humanized anti-GITR antibodies, which may comprise the heavy chain framework of IGHV4-59*01 and/or a light chain framework of IGKV3-11*01, may comprise one or more amino acid residue variations in one or more CDR regions as relative to the corresponding CDR regions of the murine parent Lyv396. For example, the humanized antibody may comprise up to 5 (e.g., up to 4, 3, 2, or 1) amino acid residues in the three heavy chain CDRs collectively. In other examples, the humanized antibody may comprise up to 5 (e.g., up to 4, 3, 2, or 1) amino acid residues in the three light chain CDRs collectively. In yet other examples, the humanized antibody may comprise up to 8 (e.g., up to 7, 6, 5, 4, 3, 2, or 1) amino acid residues in the three heavy chain CDRs and the three light chain CDRs collectively.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, any of the humanized anti-GITR antibodies may comprise the same framework as those encoded by the human acceptor germline V_{H} and/or V_{L} gene. In other embodiments, the framework region of the humanized antibodies may comprise one or more mutations relative to those encoded by the human acceptor germline V_{H} and/or V_{L} gene. For example, one or more positions in the framework region of the V_{H} and/or V_{L} chain of a humanized antibody may contain one or more back mutations, which refer to changing a residue in the human acceptor germline gene back to the residue at the corresponding position of the murine parent. For example, humanized antibodies derived from murine parent clone Lyv392 may comprise mutations (e.g., back mutations) at one or more of positions E1 (e.g., E1D), 12 (e.g., I2T), 148 (e.g., I48V), V85 (e.g., V85T), and/or Y87 (e.g., Y87F) in the light chain framework regions. In some examples, the humanized anti-GITR antibodies disclosed herein may comprise any of the heavy chain and light chain CDRs disclosed herein (e.g., any of the CDR combinations provided in Example 3 below). In addition, such a humanized anti-GITR antibody may comprise a heavy chain framework at least 80% (e.g., at least 85%, 90%, 95% or above) identical to the heavy chain framework region of IGHV4-59*01. Alternatively or in addition, the humanized anti-GITR antibody may comprise a light chain framework at least 80% (e.g., at least 85%, 90%, 95% or above) identical to the light chain framework region of IGKV3-11*01.

Any of the anti-GITR antibodies described herein may be a full-length antibody, which contains two heavy chains and two light chains, each including a variable domain and a constant domain. Alternatively, the heavy chain constant region of the antibodies described herein may comprise a single domain (e.g., CH1, CH2, or CH3) or a combination of any of the single domains. Antibody heavy and light chain constant regions are well known in the art, e.g., those provided in the IMGT database (www.imgt.org) or at www.vbase2.org/vbstat.php., both of which are incorporated by reference herein.

Alternatively, the antibodies disclosed herein can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)2 fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

In some embodiments, the anti-GITR antibody is TM676 disclosed in Example 3 below or a functional variant derived therefrom. TM676 or a functional variant thereof may comprise V_{H} and V_{L} chains fused to a human heavy chain constant region and a human light cian constant region, respectively. The human heavy chain constant region may be from an IgG molecule and/or the human light chain constant region may be from a kappa chain. The heavy chain constant domain may be derived from a suitable Ig isoform, for example, a human IgG1, IgG2, or IgG4 molecule. In some embodiments, the constant domain may comprise one or more mutations in the Fc region to enhance or reduce binding affinity and/or binding specificity to an Fc receptor. Examples are provided herein or disclosed in WO/2018/183520 and PCT/US2019/053505 (filed on Sep. 27, 2019), the relevant disclosures of each of which are incorporated by reference for the purpose and subject matter referenced herein. Such a recombinant antibody may further comprise the same light chain variable region of TM676 fused to a human light chain constant region, for example, a kappa chain constant region.

In some embodiments, the anti-GITR antibody is TM677 disclosed in Example 3 below or a functional variant derived therefrom. TM677 or a functional variant thereof may comprise V_{H} and V_{L} chains fused to a human heavy chain constant region and a human light cian constant region, respectively. The human heavy chain constant region may be from an IgG molecule and/or the human light chain constant region may be from a kappa chain. The heavy chain constant domain may be derived from a suitable Ig isoform, for example, a human IgG1, IgG2, or IgG4 molecule. In some embodiments, the constant domain may comprise one or more mutations in the Fc region to enhance or reduce binding affinity and/or binding specificity to an Fc receptor. Examples are provided herein or disclosed in WO/2018/183520 and PCT/US2019/053505 (filed on Sep. 27, 2019), the relevant disclosures of each of which are incorporated by reference for the purpose and subject matter referenced herein. Such a recombinant antibody may further comprise the same light chain variable region of TM677 fused to a human light chain constant region, for example, a kappa chain constant region.

In some embodiments, the anti-GITR antibody is TM685 disclosed in Example 3 below or a functional variant derived therefrom. TM685 or a functional variant thereof may comprise V_{H} and V_{L} chains fused to a human heavy chain constant region and a human light cian constant region, respectively. The human heavy chain constant region may be from an IgG molecule and/or the human light chain constant region may be from a kappa chain. The heavy chain constant domain may be derived from a suitable Ig isoform, for example, a human IgG1, IgG2, or IgG4 molecule. In some embodiments, the constant domain may comprise one or more mutations in the Fc region to enhance or reduce binding affinity and/or binding specificity to an Fc receptor. Examples are provided herein or disclosed in WO/2018/183520 and PCT/US2019/053505 (filed on Sep. 27, 2019), the relevant disclosures of each of which are incorporated by reference for the purpose and subject matter referenced herein. Such a recombinant antibody may further comprise the same light chain variable region of TM685 fused to a human light chain constant region, for example, a kappa chain constant region.

Exemplary anti-GITR antibodies and humanized versions thereof are provided in Example 3 below, which are also within the scope of the present disclosure.

### III. Methods for Antibody Preparation

Any of the antibodies, including bi-specific antibodies, as described herein can be made by any method known in the art. See, for example, Harlow and Lane, (1998) Antibodies: A Laboratory Manual, Cold Spring Harbor Laboratory, New York. Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a target antigen as disclosed herein can be identified from the library following routine procedures.

In some examples, any of the antibodies, including bi-specific antibodies as disclosed herein can be prepared by recombinant technology as exemplified below.

Nucleic acids encoding the heavy and light chain of the antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters (Brown, M. et al., Cell, 49:603-612 (1987)), those using the tetracycline repressor (tetR) (Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an antibody (including bi-specific antibody) as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding a first chain (e.g., a heavy chain) of the antibody and the other encoding a second chain (e.g., a light chain) of the antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the first chain (e.g., the heavy chain), the second chain (e.g., the light chain), or both of an antibody as described herein, vectors (e.g., expression vectors) containing such; and host cells comprising the vectors are within the scope of the present disclosure.

### IV. Pharmaceutical Compositions

Any of the antibodies, including bi-specific antibodies disclosed herein, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### V. Therapeutic Applications

Any of the anti-CD137/PD-1 bi-specific antibodies, anti-CD137/PD-L1 bi-specific antibodies, anti-CD137/GITR bi-specific antibodies, anti-CD137/CD40 bi-specific antibodies, anti-CD137/OX40 bi-specific antibodies, as well as any of the anti-GITR antibodies disclosed herein, may be used in clinical settings (e.g., therapeutic or diagnostic) or in non-clinical settings (e.g., for research purposes).

In some aspects, provided herein are methods of using any of the antibodies disclosed herein for modulating immune responses or for treating a targeting disease in a subject in need of the treatment. To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as a cancer or an immune disorder such as an autoimmune disease.

Examples of cancers include, but are not limited to, breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia, e.g., B Cell CLL; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

A subject having a target cancer can be identified by routine medical examination, e.g., laboratory tests, organ functional tests, CT scans, ultrasounds, and/or genetic testing. In some embodiments, the subject to be treated by the method described herein may be a human cancer patient who has undergone or is subjecting to an anti-cancer therapy, for example, chemotherapy, radiotherapy, immunotherapy, or surgery.

Immune disorders refer to a dysfunction of the immune system. Examples include autoimmune diseases, immunodeficiencies, or allergies. In some embodiments, the target disease for treatment is an autoimmune disease. Examples include, but are not limited to, rheumatoid arthritis (RA), systemic lupus erythematosus (SLE), Myasthenia Gravis (MG), Graves' Disease, Idiopathic Thrombocytopenia Purpura (ITP), Guillain-Barre Syndrome, autoimmune myocarditis, Membrane Glomerulonephritis, Hyper IgM syndrome, diabetes mellitus, Type I or Type II diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, juvenile onset diabetes, and immune responses associated with delayed hypersensitivity mediated by cytokines, T-lymphocytes typically found in tuberculosis, sarcoidosis, and polymyositis, polyarteritis, cutaneous vasculitis, pemphigus, pemphigold, Goodpasture's syndrome, Kawasaki's disease, systemic sclerosis, anti-phospholipid syndrome, Sjogren's syndrome, graft-versus-host (GVH) disease, and immune thrombocytopenia.

A subject having a target autoimmune disease can be identified by routine medical examination, e.g., presence of antinuclear antibodies, anti-mitochondrial autoantibodies, anti-neutrophil cytoplasmic antibody, anti-phospholipid antibodies, anti-citrullinated peptide (anti-CCP), anti-rheumatoid factor, immunoglobulin A, C-reactive protein test, complement test, erythrocyte sedimentation rate (ESR) test, blood clotting profile, and protein electrophoresis/immunofixation electrophoresis, and/or genetic testings. In some embodiments, the subject to be treated by the method described herein may be a human subject with an autoimmune disease who has undergone or is subjecting to an autoimmune disease treatment, for example, immunosuppressive mediation, hormone replacement therapy, blood transfusions, anti-inflammatory medication, and/or pain medication.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.003 to 5.00 mg/kg may be administered. In some examples, the dosage of the antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents. Treatment efficacy for a target disease/disorder can be assessed by methods well-known in the art.

When any of the antibodies described herein is used for treating a cancer, it can be combined with an anti-cancer therapy, for example, those known in the art. Additional anti-cancer therapy includes chemotherapy, surgery, radiation, immunotherapy, gene therapy, and so forth.

Alternatively, the treatment of the present disclosure can be combined with a chemotherapeutic agent, for example, pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

When any of the antibodies described herein is for use in treating an immune disorder, it can be co-used with other immunomodulatory treatments such as, e.g., therapeutic vaccines (including but not limited to GVAX, DC-based vaccines, etc.), or checkpoint inhibitors (including but not limited to agents that block CTLA4, PD1, LAG3, TIM3, etc.). In some instances, the antibody can be combined with another therapy for autoimmune diseases. Examples include, but are not limited to, intravenous Ig therapy; nonsteroidal anti-inflammatory drugs (NSAID); corticosteroids; cyclosporins, rapamycins, ascomycins; cyclophosphamide; azathioprene; methotrexate; brequinar; FTY 720; leflunomide; mizoribine; mycophenolic acid; mycophenolate mofetil; 15-deoxyspergualine; an immunosuppressive agent, or an adhesion molecule inhibitor.

For examples of additional useful agents see also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

When a second therapeutic agent is used, such an agent can be administered simultaneously or sequentially (in any order) with the therapeutic agent described herein. When co-administered with an additional therapeutic agent, suitable therapeutically effective dosages for each agent may be lowered due to the additive action or synergy.

### VI. Kits Comprising Antibodies Disclosed Herein

The present disclosure also provides kits for use in treating or alleviating a target disease, such as cancer or immune disorders as described herein. Such kits can include one or more containers comprising an anti-GITR antibody, anti-CD137/PD-1 bi-specific antibody, anti-CD137/PD-L1 bi-specific antibody, anti-CD137/GITR bi-specific antibody, anti-CD137/CD40 bi-specific antibody, and/or anti-CD137/OX40 bi-specific antibody, e.g., any of those described herein, and optionally a second therapeutic agent to be co-used with the antibody, which is also described herein.

In some embodiments, the kit can comprise instructions for use in accordance with any of the methods described herein. The included instructions can comprise a description of administration of the antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds (1985; Transcription and Translation (B. D. Hames & S. J. Higgins, eds. (1984; Animal Cell Culture (R. I. Freshney, ed. (1986; Immobilized Cells and Enzymes (lRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Anti-PD-1/CD137 Bi-Specific Antibodies

Anti-PD-1/CD137 bi-specific antibodies were produced and characterized using parent anti-CD137 antibody clone Ly1630 and parent anti-PD-1 antibody clone Ly516. Ly516v (used as a control in some assays) is a variant, which differs from Ly516 by one amino acid residue substitution (P96T in LC CDR3). All these antibodies are humanized antibodies. The amino acid sequences of the V_{H} and the V_{L} of the parent clones are provided below. The heavy chain and light chain complementary determining regions determined by the Kabat scheme are in boldface.

Ly1630
VH (SEQ ID NO: 1):
QVQLVQSGAEVKKPGASVKVSCKASGYTFAGFEMHWVRQAPGQGLEWMGA
IDPKTGGTDYNQKFKDRVTMTRDTSISTAYMELSRLRSDDTAVYYCARDL
GYFDVWGQGTLVTVSS
VL (SEQ ID NO: 2):
DIQMTQSPSSLSASVGDRVTITCRASQDIRSNLNWYQQKPGGAVKLLIYY
TSRLHSGVPSRFSGSGSGTDYTLTISSLQPEDFATYFCQQSEKLPRTFGG
GTKVEIR
Ly516
VH (SEQ ID NO: 3):
QVTLKESGPALVKPTQTLTLTCTFSGFSLSTSGTCVSWIRQPPGKALEWL
ATICWEDSKGYNPSLKSRLTISKDTSKNQAVLTMTNMDPVDTATYYCARR
EDSGYFWFPYWGQGTLVTVSS
VL (SEQ ID NO: 4):
NIQMTQSPSSLSASVGDRVTITCKAGQNVNNYLAWYQQKPGKAPKVLIFN
ANSLQTGVPSRFSGSGSGTDFTLTISSLQPEDFATYYCQQYNSWPTFGGG
TKVEIK

cDNAs encoding the heavy chain variable region (V_{H}) and the light chain variable region (V_{L}) of the parent clones were used as the starting materials for making the anti-PD-1/anti-CD-137 bi-specific antibodies. CHO-cell transient expression was carried out with plasmids configured for expressing the polypeptide chains of the bi-specific antibodies. These resultant bispecific antibodies were purified by protein A affinity chromatography.

The amino acid sequences of the polypeptides of exemplary bi-specific antibodies derived from Ly1630 and Ly516 are provided below:

Ly456
First polypeptide (SEQ ID NO: 5):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSGGGGSGGGGSDIQMTQSPSSLSAS
VGDRVTITCRASQDIRSNLNWYQQKPGGAVKLLIYYTSRLHSGVPSRFSGSGSGTDYTLTISSLQPEDF
ATYFCQQSEKLPRTFGGGTKVEIRGGGGSGGGGSGGGGSGGGGSQVQLVQSGAEVKKPGASVKVSCKAS
GYTFAGFEMHWVRQAPGQGLEWMGAIDPKTGGTDYNQKFKDRVTMTRDTSISTAYMELSRLRSDDTAVY
YCARDLGYFDVWGQGTLVTVSS
Second polypeptide (SEQ ID NO: 6)
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGEC
Ly457
First polypeptide (SEQ ID NO: 7):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSGGGGSGGGGSQVQLVQSGAEVKKP
GASVKVSCKASGYTFAGFEMHWVRQAPGQGLEWMGAIDPKTGGTDYNQKFKDRVTMTRDTSISTAYMEL
SRLRSDDTAVYYCARDLGYFDVWGQGTLVTVSSGGGGSGGGGSGGGGSGGGGSDIQMTQSPSSLSASVG
DRVTITCRASQDIRSNLNWYQQKPGGAVKLLIYYTSRLHSGVPSRFSGSGSGTDYTLTISSLQPEDFAT
YFCQQSEKLPRTFGGGTKVEIR
Second polypeptide (SEQ ID NO: 6): same as the second polypeptide of Ly456
Ly458
First polypeptide (SEQ ID NO: 8):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
Second polypeptide (SEQ ID NO: 9):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSDIQMTQSPSSLSASVGDRVTITCRASQDIRSNLNWYQQKPGGA
VKLLIYYTSRLHSGVPSRFSGSGSGTDYTLTISSLQPEDFATYFCQQSEKLPRTFGGGTKVEIRGGGGS
GGGGSGGGGGGGGSQVQLVQSGAEVKKPGASVKVSCKASGYTFAGFEMHWVRQAPGQGLEWMGAIDPK
TGGTDYNQKFKDRVTMTRDTSISTAYMELSRLRSDDTAVYYCARDLGYFDVWGQGTLVTVSS
Ly459
First polypeptide (SEQ ID NO: 8): same as the first polypeptide of Ly458.
Second polypeptide (SEQ ID NO: 10):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSQVQLVQSGAEVKKPGASVKVSCKASGYTFAGFEMHWVRQAPGQ
GLEWMGAIDPKTGGTDYNQKFKDRVTMTRDTSISTAYMELSRLRSDDTAVYYCARDLGYFDVWGQGTLV
TVSSGGGGSGGGGSGGGGSGGGGSDIQMTQSPSSLSASVGDRVTITCRASQDIRSNLNWYQQKPGGAVK
LLIYYTSRLHSGVPSRFSGSGSGTDYTLTISSLQPEDFATYFCQQSEKLPRTFGGGTKVEIR
Ly460
First polypeptide (SEQ ID NO: 11):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSGGGGSGGGGSDIQMTQSPSSLSAS
VGDRVTITCRASQDIRSNLNWYQQKPGGAVKLLIYYTSRLHSGVPSRFSGSGSGTDYTLTISSLQPEDF
ATYFCQQSEKLPRTFGGGTKVEIRRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSFNRGEC
Second polypeptide (SEQ ID NO: 6): same as the second polypeptide of Ly456
Third polypeptide (SEQ ID NO: 12)
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDK
Ly461
First polypeptide (SEQ ID NO: 13):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSNIQMTQSPSSLSASVGDRV
TITCKAGQNVNNYLAWYQQKPGKAPKVLIFNANSLQTGVPSRFSGSGSGTDFTLTISSLQPEDFATYYC
QQYNSWPTFGGGTKVEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Second polypeptide (SEQ ID NO: 14):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGEC
Third polypeptide (SEQ ID NO: 15)
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDK
Ly510
First polypeptide (SEQ ID NO: 16):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPCSRS
TSESTAALGCLVKDYFPEPVTVSWNSGALTSGVHTFPAVLQSSGLYSLSSVVTVPSSNFGTQTYTCNVD
HKPSNTKVDKTVERKCCVECPPCPAPPVAPSVFLFPPKPKDTLMISRTPEVTCVVVDVSHEDPEVQFNW
YVDGVEVHNAKTKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFFLYSKLTVDKS
RWQQGNVFSCSVMHEALHNHYTQKSLSLSPGGGGGSGGGGSGGGGSGGGGSDIQMTQSPSSLSASVGDR
VTITCRASQDIRSNLNWYQQKPGGAVKLLIYYTSRLHSGVPSRFSGSGSGTDYTLTISSLQPEDFATYF
CQQSEKLPRTFGGGTKVEIRGGGGSGGGGSGGGGSGGGGSQVQLVQSGAEVKKPGASVKVSCKASGYTF
AGFEMHWVRQAPGQGLEWMGAIDPKTGGTDYNQKFKDRVTMTRDTSISTAYMELSRLRSDDTAVYYCAR
DLGYFDVWGQGTLVTVSS
Second polypeptide (SEQ ID NO: 6): same as the second polypeptide of Ly456
Ly511
First polypeptide (SEQ ID NO: 17):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPCSRS
TSESTAALGCLVKDYFPEPVTVSWNSGALTSGVHTFPAVLQSSGLYSLSSVVTVPSSNFGTQTYTCNVD
HKPSNTKVDKTVERKCCVECPPCPAPPVAPSVFLFPPKPKDTLMISRTPEVTCVVVDVSHEDPEVQFNW
YVDGVEVHNAKTKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFFLYSKLTVDKS
RWQQGNVFSCSVMHEALHNHYTQKSLSLSPGGGGGSGGGGSGGGGSGGGGSQVQLVQSGAEVKKPGASV
KVSCKASGYTFAGFEMHWVRQAPGQGLEWMGAIDPKTGGTDYNQKFKDRVTMTRDTSISTAYMELSRLR
SDDTAVYYCARDLGYFDVWGQGTLVTVSSGGGGSGGGGSGGGGSGGGGSDIQMTQSPSSLSASVGDRVT
ITCRASQDIRSNLNWYQQKPGGAVKLLIYYTSRLHSGVPSRFSGSGSGTDYTLTISSLQPEDFATYFCQ
QSEKLPRTFGGGTKVEIR
Second polypeptide (SEQ ID NO: 6): same as the second polypeptide of Ly456
Ly512
First polypeptide (SEQ ID NO: 18):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPCSRS
TSESTAALGCLVKDYFPEPVTVSWNSGALTSGVHTFPAVLQSSGLYSLSSVVTVPSSNFGTQTYTCNVD
HKPSNTKVDKTVERKCCVECPPCPAPPVAPSVFLFPPKPKDTLMISRTPEVTCVVVDVSHEDPEVQFNW
YVDGVEVHNAKTKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFFLYSKLTVDKS
RWQQGNVFSCSVMHEALHNHYTQKSLSLSPGK
Second polypeptide (SEQ ID NO: 9): same as the second polypeptide of Ly458.
Ly513
First polypeptide (SEQ ID NO: 18): same as the first polypeptide of Ly512
Second polypeptide (SEQ ID NO: 10): same as the second polypeptide of Ly459
Ly514
First polypeptide (SEQ ID NO: 19):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPCSRS
TSESTAALGCLVKDYFPEPVTVSWNSGALTSGVHTFPAVLQSSGLYSLSSVVTVPSSNFGTQTYTCNVD
HKPSNTKVDKTVERKCCVECPPCPAPPVAPSVFLFPPKPKDTLMISRTPEVTCVVVDVSHEDPEVQFNW
YVDGVEVHNAKTKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFFLYSKLTVDKS
RWQQGNVFSCSVMHEALHNHYTQKSLSLSPGGGGGSGGGGSGGGGSGGGGSDIQMTQSPSSLSASVGDR
VTITCRASQDIRSNLNWYQQKPGGAVKLLIYYTSRLHSGVPSRFSGSGSGTDYTLTISSLQPEDFATYF
CQQSEKLPRTFGGGTKVEIRRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
Second polypeptide: (SEQ ID NO: 6): same as the second polypeptide of Ly456
Third polypeptide: (SEQ ID NO: 12): same as the third polypeptide of Ly460
Ly515
First polypeptide (SEQ ID NO: 20):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPCSRSTSEST
AALGCLVKDYFPEPVTVSWNSGALTSGVHTFPAVLQSSGLYSLSSVVTVPSSNFGTQTYTCNVDHKPSN
TKVDKTVERKCCVECPPCPAPPVAPSVFLFPPKPKDTLMISRTPEVTCVVVDVSHEDPEVQFNWYVDGV
EVHNAKTKPREEQFNSTFRVVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSDGSFFLYSKLTVDKSRWQQG
NVFSCSVMHEALHNHYTQKSLSLSPGGGGGSGGGGSGGGGSGGGGSNIQMTQSPSSLSASVGDRVTITC
KAGQNVNNYLAWYQQKPGKAPKVLIFNANSLQTGVPSRFSGSGSGTDFTLTISSLQPEDFATYYCQQYN
SWPTFGGGTKVEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
Second polypeptide: (SEQ ID NO: 14): same as the second polypeptide of Ly461
Third polypeptide: (SEQ ID NO: 15): same as the third polypeptide of Ly461
Ly555
First polypeptide (SEQ ID NO: 21):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKGGGGSQVQLVQSGAEVKKPGASVKVSCKASGY
TFAGFEMHWVRQAPGQGLEWMGAIDPKTGGTDYNQKFKDRVTMTRDTSISTAYMELSRLRSDDTAVYYC
ARDLGYFDVWGQGTLVTVSSDKTHTCPPCPAPELLGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPG
Second polypeptide (SEQ ID NO: 22):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRGGGGSQVTLKESGPALVKPTQTLTLTCTFSG
FSLSTSGTCVSWIRQPPGKALEWLATICWEDSKGYNPSLKSRLTISKDTSKNQAVLTMTNMDPVDTATY
YCARREDSGYFWFPYWGQGTLVTVSS
Ly556
First polypeptide (SEQ ID NO: 23):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRGGGGSQVTLKESGPALVKPTQTLTLTCTFSG
FSLSTSGTCVSWIRQPPGKALEWLATICWEDSKGYNPSLKSRLTISKDTSKNQAVLTMTNMDPVDTATY
YCARREDSGYFWFPYWGQGTLVTVSSDKTHTCPPCPAPELLGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG
Second polypeptide (SEQ ID NO: 24):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKGGGGSQVQLVQSGAEVKKPGASVKVSCKASGY
TFAGFEMHWVRQAPGQGLEWMGAIDPKTGGTDYNQKFKDRVTMTRDTSISTAYMELSRLRSDDTAVYYC
ARDLGYFDVWGQGTLVTVSS
Ly557
First polypeptide (SEQ ID NO: 25):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKGGGSGGGGQVQLVQSGAEVKKPGASVKVSCKA
SGYTFAGFEMHWVRQAPGQGLEWMGAIDPKTGGTDYNQKFKDRVTMTRDTSISTAYMELSRLRSDDTAV
YYCARDLGYFDVWGQGTLVTVSSGGCGGGEVAACEKEVAALEKEVAALEKEVAALEKLEPKSADKTHTC
PPCPAPEAAGGPSVFLFPPKPKDTLYITREPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALH
NHYTQKSLSLSPG
Second polypeptide (SEQ ID NO: 26):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRGGGSGGGGQVTLKESGPALVKPTQTLTLTCT
FSGFSLSTSGTCVSWIRQPPGKALEWLATICWEDSKGYNPSLKSRLTISKDTSKNQAVLTMTNMDPVDT
ATYYCARREDSGYFWFPYWGQGTLVTVSSGGCGGGKVAACKEKVAALKEKVAALKEKVAALKE
Ly558
First polypeptide (SEQ ID NO: 27):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRGGGSGGGGQVTLKESGPALVKPTQTLTLTCT
FSGFSLSTSGTCVSWIRQPPGKALEWLATICWEDSKGYNPSLKSRLTISKDTSKNQAVLTMTNMDPVDT
ATYYCARREDSGYFWFPYWGQGTLVTVSSGGCGGGEVAACEKEVAALEKEVAALEKEVAALEKLEPKSA
DKTHTCPPCPAPEAAGGPSVFLFPPKPKDTLYITREPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQGNVFSCSV
MHEALHNHYTQKSLSLSPG
Second polypeptide (SEQ ID NO: 28):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKGGGSGGGGQVQLVQSGAEVKKPGASVKVSCKA
SGYTFAGFEMHWVRQAPGQGLEWMGAIDPKTGGTDYNQKFKDRVTMTRDTSISTAYMELSRLRSDDTAV
YYCARDLGYFDVWGQGTLVTVSSGGCGGGKVAACKEKVAALKEKVAALKEKVAALKE
Ly666
First polypeptide (SEQ ID NO: 29):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSQVQLVQSGAEVKKPGAS
VKVSCKASGYTFAGFEMHWVRQAPGQGLEWMGAIDPKTGGTDYNQKFKDRVTMTRDTSISTAYMELSRL
RSDDTAVYYCARDLGYFDVWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVEPKSCDKTHT
CPPCPAPELLGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALH
NHYTQKSLSLSPGK
Second polypeptide (SEQ ID NO: 30):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKDIQMTQSPSSLSASVGDRVTITCRASQDIRSN
LNWYQQKPGGAVKLLIYYTSRLHSGVPSRFSGSGSGTDYTLTISSLQPEDFATYFCQQSEKLPRTFGGG
TKVEIRRTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
Ly667
First polypeptide (SEQ ID NO: 31):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPQVQLVQSGAEV
KKPGASVKVSCKASGYTFAGFEMHWVRQAPGQGLEWMGAIDPKTGGTDYNQKFKDRVTMTRDTSISTAY
MELSRLRSDDTAVYYCARDLGYFDVWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKS
CDKTHTCPPCPAPELLGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQGNVFSCSV
MHEALHNHYTQKSLSLSPGK
Second polypeptide (SEQ ID NO: 32):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPDIQMTQSPSSLSASVGDRVTITCRAS
QDIRSNLNWYQQKPGGAVKLLIYYTSRLHSGVPSRFSGSGSGTDYTLTISSLQPEDFATYFCQQSEKLP
RTFGGGTKVEIRRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Ly668
First polypeptide (SEQ ID NO: 33):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPQVQL
VQSGAEVKKPGASVKVSCKASGYTFAGFEMHWVRQAPGQGLEWMGAIDPKTGGTDYNQKFKDRVTMTRD
TSISTAYMELSRLRSDDTAVYYCARDLGYFDVWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVD
KKVEPKSCDKTHTCPPCPAPELLGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
Second polypeptide (SEQ ID NO: 34):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPSVFIFPPDIQMTQSPSSLSASVGDRV
TITCRASQDIRSNLNWYQQKPGGAVKLLIYYTSRLHSGVPSRFSGSGSGTDYTLTISSLQPEDFATYFC
QQSEKLPRTFGGGTKVEIRRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Ly669
First polypeptide (SEQ ID NO: 35):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSQVTLKESGPALVKPTQTLTLTC
TFSGFSLSTSGTCVSWIRQPPGKALEWLATICWEDSKGYNPSLKSRLTISKDTSKNQAVLTMTNMDPVD
TATYYCARREDSGYFWFPYWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVEPKSCDKTHT
CPPCPAPELLGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALH
NHYTQKSLSLSPGK
Second polypeptide (SEQ ID NO: 36):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRNIQMTQSPSSLSASVGDRVTITCKAGQNVNN
YLAWYQQKPGKAPKVLIFNANSLQTGVPSRFSGSGSGTDFTLTISSLQPEDFATYYCQQYNSWPTFGGG
TKVEIKRTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
Ly670
First polypeptide (SEQ ID NO: 37):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPQVTLKESGPALVKPTQ
TLTLTCTFSGFSLSTSGTCVSWIRQPPGKALEWLATICWEDSKGYNPSLKSRLTISKDTSKNQAVLTMT
NMDPVDTATYYCARREDSGYFWFPYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKS
CDKTHTCPPCPAPELLGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQGNVFSCSV
MHEALHNHYTQKSLSLSPGK
Second polypeptide (SEQ ID NO: 38):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPNIQMTQSPSSLSASVGDRVTITCKA
GQNVNNYLAWYQQKPGKAPKVLIFNANSLQTGVPSRFSGSGSGTDFTLTISSLQPEDFATYYCQQYNSW
PTFGGGTKVEIKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Ly671
First polypeptide (SEQ ID NO: 39):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPQVTLKESGP
ALVKPTQTLTLTCTFSGFSLSTSGTCVSWIRQPPGKALEWLATICWEDSKGYNPSLKSRLTISKDTSKN
QAVLTMTNMDPVDTATYYCARREDSGYFWFPYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVD
KKVEPKSCDKTHTCPPCPAPELLGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
Second polypeptide (SEQ ID NO: 40):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPNIQMTQSPSSLSASVGDR
VTITCKAGQNVNNYLAWYQQKPGKAPKVLIFNANSLQTGVPSRFSGSGSGTDFTLTISSLQPEDFATYY
CQQYNSWPTFGGGTKVEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Ly672
First polypeptide (SEQ ID NO: 41):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECQVQLVQSGAEVKKPGASVKVSCKASGYTFAGFEMHWVRQAPGQGLEWMGAIDPKTGGTDYNQK
FKDRVTMTRDTSISTAYMELSRLRSDDTAVYYCARDLGYFDVWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
Second polypeptide (SEQ ID NO: 124):
QVTLKESGPALVKPTQTLTLTCTFSGFSLSTSGTCVSWIRQPPGKALEWLATICWEDSKGYNPSLKSRL
TISKDTSKNQAVLTMTNMDPVDTATYYCARREDSGYFWFPYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSC
Third polypeptide (SEQ ID NO: 15): same as the second polypeptide of Ly461
Ly673
First polypeptide (SEQ ID NO: 42):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGSGQVQLVQSGAEVKKPGASVKVSCKASGYTFAGFEMHWVRQAPGQGLEWMGAIDPKTGGTDY
NQKFKDRVTMTRDTSISTAYMELSRLRSDDTAVYYCARDLGYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
Second polypeptide (SEQ ID NO: 124): same as the second polypeptide of Ly672
Third polypeptide (SEQ ID NO: 14): same as the second polypeptide of Ly461
Ly674
First polypeptide (SEQ ID NO: 43):
NIQMTQSPSSLSASVGDRVTITCKAGQNVNNYLAWYQQKPGKAPKVLIFNANSLQTGVPSRFSGSGSGT
DFTLTISSLQPEDFATYYCQQYNSWPTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGGGGSGSQVQLVQSGAEVKKPGASVKVSCKASGYTFAGFEMHWVRQAPGQGLEWMGAIDPKTG
GTDYNQKFKDRVTMTRDTSISTAYMELSRLRSDDTAVYYCARDLGYFDVWGQGTLVTVSSASTKGPSVF
PLAPSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
Second polypeptide (SEQ ID NO: 124): same as the second polypeptide of Ly672
Third polypeptide (SEQ ID NO: 14): same as the second polypeptide of Ly461
Ly675
First polypeptide (SEQ ID NO: 44):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECQVTLKESGPALVKPTQTLTLTCTFSGFSLSTSGTCVSWIRQPPGKALEWLATICWEDSKGYN
PSLKSRLTISKDTSKNQAVLTMTNMDPVDTATYYCARREDSGYFWFPYWGQGTLVTVSSASTKGPSVFP
LAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
Second polypeptide (SEQ ID NO: 45):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSC
Third polypeptide (SEQ ID NO: 6): same as the second polypeptide of Ly456
Ly676
First polypeptide (SEQ ID NO: 46):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGSGQVTLKESGPALVKPTQTLTLTCTFSGFSLSTSGTCVSWIRQPPGKALEWLATICWEDSK
GYNPSLKSRLTISKDTSKNQAVLTMTNMDPVDTATYYCARREDSGYFWFPYWGQGTLVTVSSASTKGPS
VFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
Second polypeptide (SEQ ID NO: 45): same as the second polypeptide of Ly675
Third polypeptide (SEQ ID NO: 6): same as the second polypeptide of Ly456
Ly677
First polypeptide (SEQ ID NO: 47):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGSQVTLKESGPALVKPTQTLTLTCTFSGFSLSTSGTCVSWIRQPPGKALEWLATICW
EDSKGYNPSLKSRLTISKDTSKNQAVLTMTNMDPVDTATYYCARREDSGYFWFPYWGQGTLVTVSSAST
KGPSVFPLAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGPSVFLFPPKPKDTLMISRTPEV
TCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Second polypeptide (SEQ ID NO: 45): same as the second polypeptide of Ly675
Third polypeptide (SEQ ID NO: 6): same as the second polypeptide of Ly456
Ly712
First polypeptide (SEQ ID NO: 48):
QVQLVQSGVEVKKPGASVKVSCKASGYTFTNYYMYWVRQAPGQGLEWMGGINPSNGGTNFNEKFKNRVT
LTTDSSTTTAYMELKSLQFDDTAVYYCARRDYRFDMGFDYWGQGTTVTVSSASTKGPSVFPLAPSSKST
SGGTAALGCLVKDYFPEPVTVSWNSGALTSGVHTFPAVLQSSGLYSLSSVVTVPSSSLGTQTYICNVNH
KPSNTKVDKKVEPKSCDKTHTCPPCPAPELLGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGGGGGSGGGGSGGGGSGGGGSDIQMTQSPSSLSASV
GDRVTITCRASQDIRSNLNWYQQKPGGAVKLLIYYTSRLHSGVPSRFSGSGSGTDYTLTISSLQPEDFA
TYFCQQSEKLPRTFGGGTKVEIRGGGGSGGGGSGGGGSGGGGSQVQLVQSGAEVKKPGASVKVSCKASG
YTFAGFEMHWVRQAPGQGLEWMGAIDPKTGGTDYNQKFKDRVTMTRDTSISTAYMELSRLRSDDTAVYY
CARDLGYFDVWGQGTLVTVSS
Second polypeptide (SEQ ID NO: 49):
EIVLTQSPATLSLSPGERATLSCRASKGVSTSGYSYLHWYQQKPGQAPRLLIYLASYLESGVPARFSGS
GSGTDFTLTISSLEPEDFAVYYCQHSRDLPLTFGGGTKVEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSS
PVTKSFNRGEC
Ly713
First polypeptide (SEQ ID NO: 50):
QVQLVQSGVEVKKPGASVKVSCKASGYTFTNYYMYWVRQAPGQGLEWMGGINPSNGGTNFNEKFKNRVT
LTTDSSTTTAYMELKSLQFDDTAVYYCARRDYRFDMGFDYWGQGTTVTVSSASTKGPSVFPLAPSSKST
SGGTAALGCLVKDYFPEPVTVSWNSGALTSGVHTFPAVLQSSGLYSLSSVVTVPSSSLGTQTYICNVNH
KPSNTKVDKKVEPKSCDKTHTCPPCPAPELLGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGGGGGSGGGGSGGGGSGGGGSQVQLVQSGAEVKKPG
ASVKVSCKASGYTFAGFEMHWVRQAPGQGLEWMGAIDPKTGGTDYNQKFKDRVTMTRDTSISTAYMELS
RLRSDDTAVYYCARDLGYFDVWGQGTLVTVSSGGGGSGGGGSGGGGSGGGGSDIQMTQSPSSLSASVGD
RVTITCRASQDIRSNLNWYQQKPGGAVKLLIYYTSRLHSGVPSRFSGSGSGTDYTLTISSLQPEDFATY
FCQQSEKLPRTFGGGTKVEIR
Second polypeptide (SEQ ID NO: 49): same as the second polypeptide of Ly712
Ly714
First polypeptide (SEQ ID NO: 51):
QVQLVQSGVEVKKPGASVKVSCKASGYTFTNYYMYWVRQAPGQGLEWMGGINPSNGGTNFNEKFKNRVT
LTTDSSTTTAYMELKSLQFDDTAVYYCARRDYRFDMGFDYWGQGTTVTVSSASTKGPSVFPLAPSSKST
SGGTAALGCLVKDYFPEPVTVSWNSGALTSGVHTFPAVLQSSGLYSLSSVVTVPSSSLGTQTYICNVNH
KPSNTKVDKKVEPKSCDKTHTCPPCPAPELLGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
Second polypeptide (SEQ ID NO: 52):
EIVLTQSPATLSLSPGERATLSCRASKGVSTSGYSYLHWYQQKPGQAPRLLIYLASYLESGVPARFSGS
GSGTDFTLTISSLEPEDFAVYYCQHSRDLPLTFGGGTKVEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSS
PVTKSFNRGECGGGGSGGGGSGGGGSGGGGSDIQMTQSPSSLSASVGDRVTITCRASQDIRSNLNWYQQ
KPGGAVKLLIYYTSRLHSGVPSRFSGSGSGTDYTLTISSLQPEDFATYFCQQSEKLPRTFGGGTKVEIR
GGGGSGGGGSGGGGSGGGGSQVQLVQSGAEVKKPGASVKVSCKASGYTFAGFEMHWVRQAPGQGLEWMG
AIDPKTGGTDYNQKFKDRVTMTRDTSISTAYMELSRLRSDDTAVYYCARDLGYFDVWGQGTLVTVSS
Ly715
First polypeptide (SEQ ID NO: 51): same as the first polypeptide of Ly714
Second polypeptide (SEQ ID NO: 53):
EIVLTQSPATLSLSPGERATLSCRASKGVSTSGYSYLHWYQQKPGQAPRLLIYLASYLESGVPARFSGS
GSGTDFTLTISSLEPEDFAVYYCQHSRDLPLTFGGGTKVEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSS
PVTKSFNRGECGGGGSGGGGSGGGGSGGGGSQVQLVQSGAEVKKPGASVKVSCKASGYTFAGFEMHWVR
QAPGQGLEWMGAIDPKTGGTDYNQKFKDRVTMTRDTSISTAYMELSRLRSDDTAVYYCARDLGYFDVWG
QGTLVTVSSGGGGSGGGGSGGGGSGGGGSDIQMTQSPSSLSASVGDRVTITCRASQDIRSNLNWYQQKP
GGAVKLLIYYTSRLHSGVPSRFSGSGSGTDYTLTISSLQPEDFATYFCQQSEKLPRTFGGGTKVEIR

### Characterization of Anti-PD-1/CD137 Bi-Specific Antibodies

(i) Binding Activity

Exemplary anti-PD-1/CD137 bi-specific antibodies were analyzed by FACS for their binding properties to human PD-1 and/or human CD137 expressed on CHO cells. Briefly, cultured cells were harvested, counted and cell viability was evaluated using the Trypan Blue exclusion method. Viable cells were then adjusted to 2×10⁶ cells per mL in PBS containing 2% BSA. 100 μL of this cell suspension were further aliquoted per well into a V-bottom 96-well plate. 50 μL of the bi-specific antibodies or corresponding IgG control were added to the cell-containing wells to obtain final concentrations of 0.1 μg/mL to 30 μg/mL. After incubation for 2 hours at 4° C., cells were centrifuged (3 min, 1000× g), washed with 250 μL/well BSA-containing FACS Stain Buffer, resuspended and incubated for an additional 1 hour at 4° C. with 100 μL/well fluorochrome-conjugated anti-IgG antibody for detection of the bisepecific antibody. Cells were then washed with 250 μL/well BSA-containing FACS Stain Buffer, resuspended in 100 μL/well FACS Stain Buffer, acquired and analyzed using a FACS machine. Binding of the bispecific antibodies to human PD-1 or human CD137 expressing CHO cells were evaluated and the mean fluorescence intensity is plotted in histograms or dot plots.

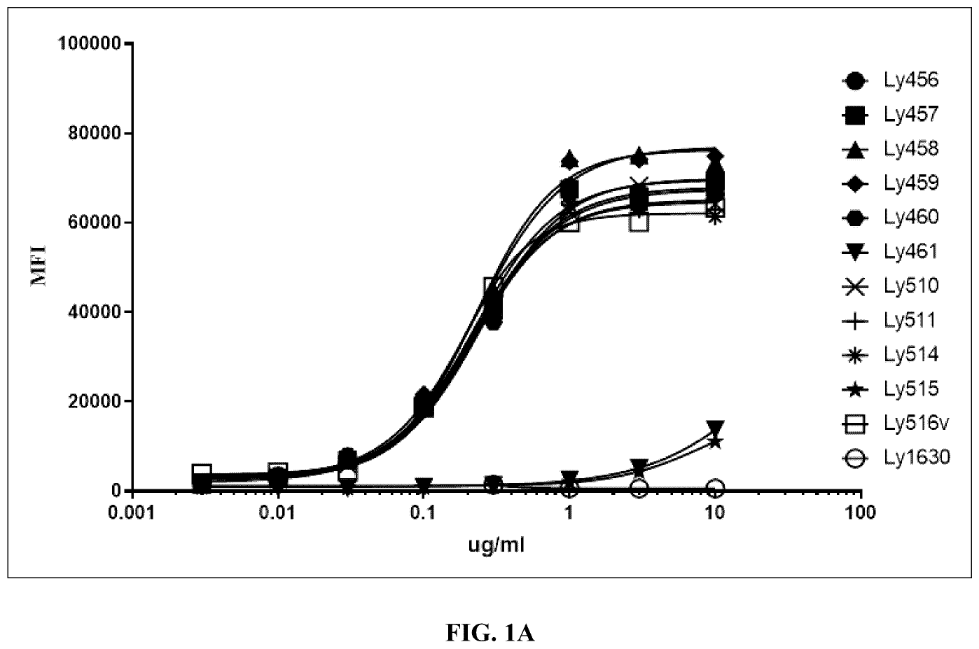

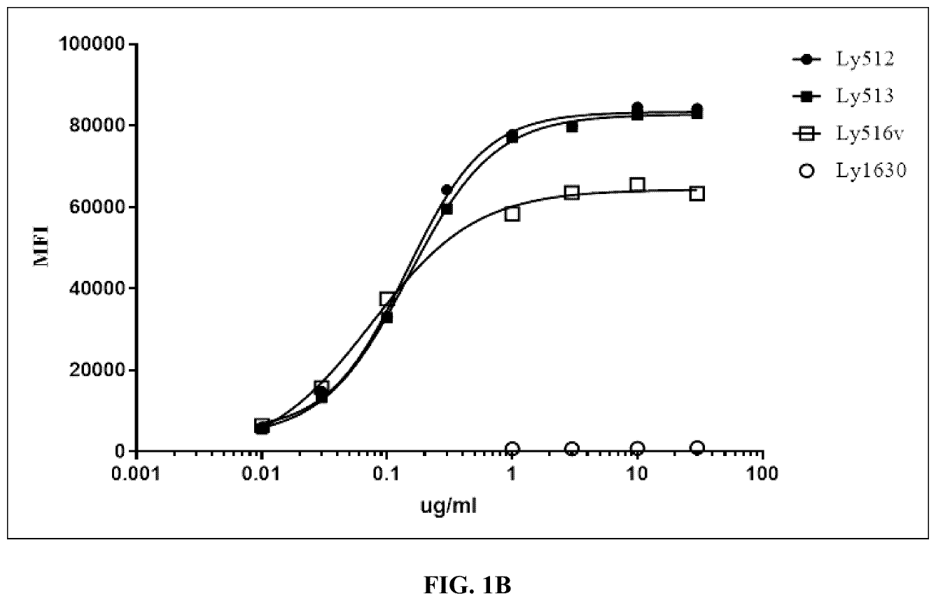

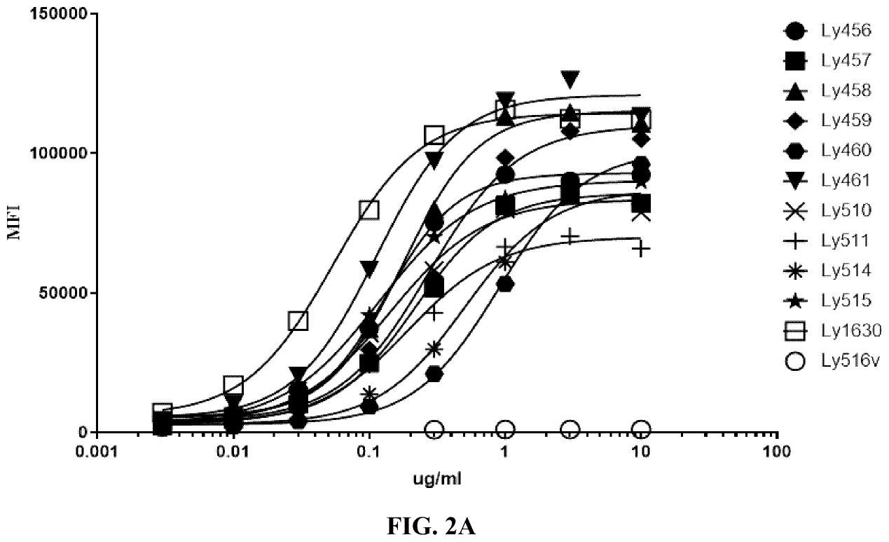

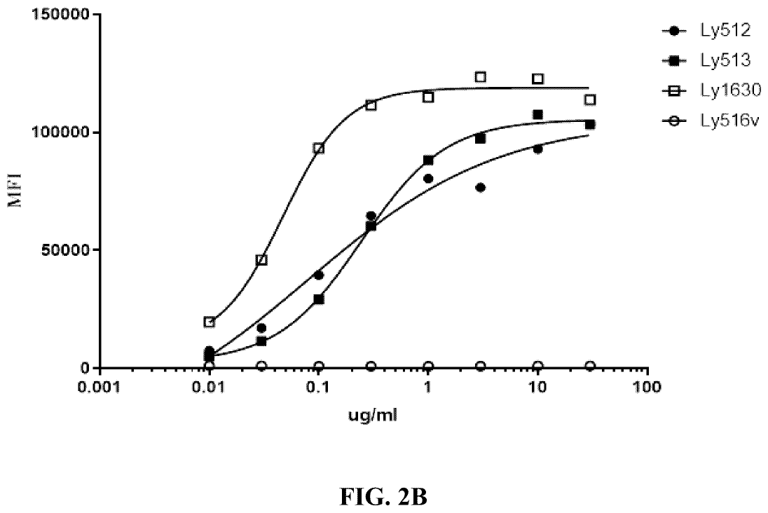

As shown in FIGS. 1A and 1B, the exemplary anti-PD-1/CD137 bi-specific antibodies comprising scFv formats of the CD137 antibodies exhibited similar binding affinity to human PD-1 over-expressed on the CHO cells relative to the anti-PD-1 parent antibody, with the exception of Ly461 and Ly515. As shown in FIGS. 2A and 2B, the bi-specific antibodies exhibited binding affinity to human CD137 expressed on CHO cells. Compared to the corresponding parental antibodies, the binding activity of bi-specific antibodies was generally weaker than that of parent Ly1630 and varied in a range, indicating the scFv format and position in these molecules impact the activity.

Exemplary anti-PD-1/CD137 bi-specific antibodies were analyzed by ELISA for their simultaneous binding to recombinant human PD-1 and human CD137. Briefly, human CD137 ECD protein (His tag) was diluted and coated onto an ELISA plate with a volume of 100 μL/well by incubation at 4° C. overnight. The next day, the plate was blocked with PBST-BSA buffer, then serially diluted samples of anti-PD-1/CD137 bi-specific antibodies were pipetted into appropriate wells at 50 μL/well, and the plate was incubated for 1 hour followed by washing. The extracellular domain (ECD) of human PD-1 protein (mouse IgG2a Fc tag) was added into the plate at 50 μL/well. After 1-hour incubation at room temperature, HRP-conjugated anti-Mouse IgG, Fc G2a specific antibody was added into the plate at 100 μL/well. The plate was incubated for 1 hour at room temperature followed by washing. TMB substrate solution was added at 100 μL/well and the color development was stopped by adding 100 uL/well Stop Solution (2N H₂SO₄). Absorbance at 450 nm and 620 nm was read by Tecan F200 Pro plate reader. GraphPad 7.0, “[Agonist] vs. response—Variable slope (four parameters)” was used to plot the binding data and calculate binding EC50 values.

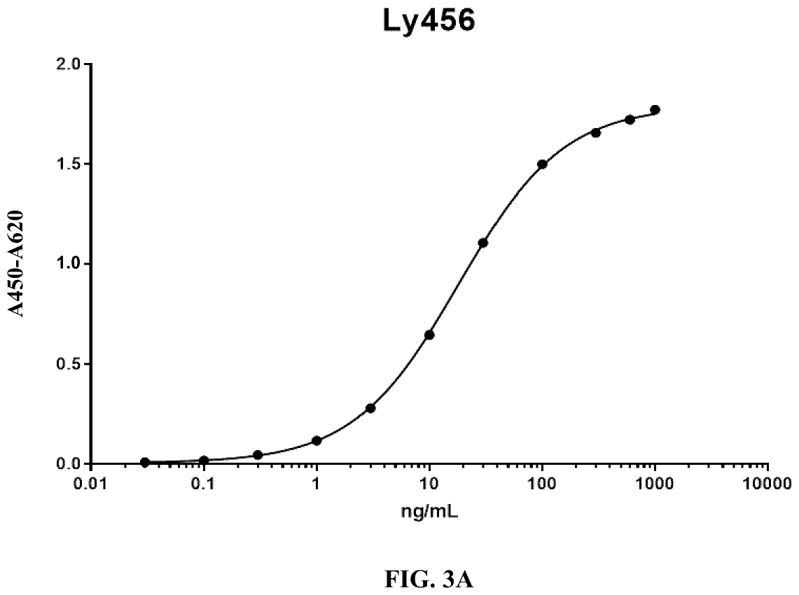

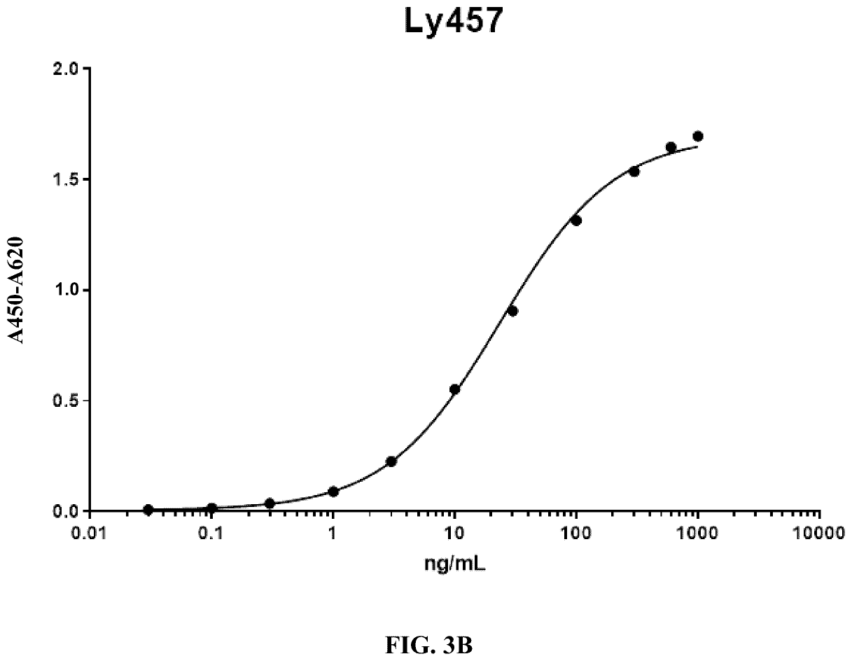

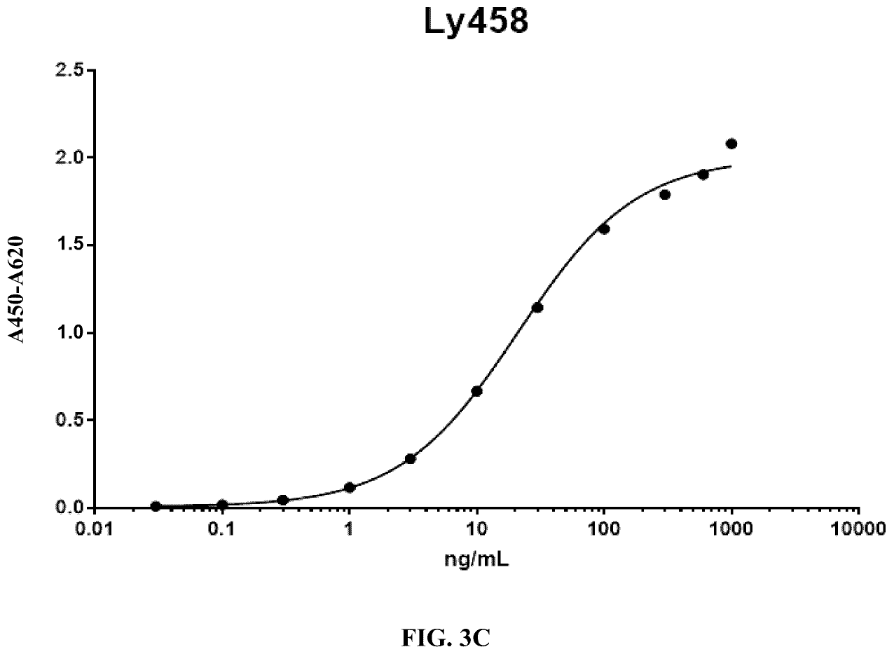

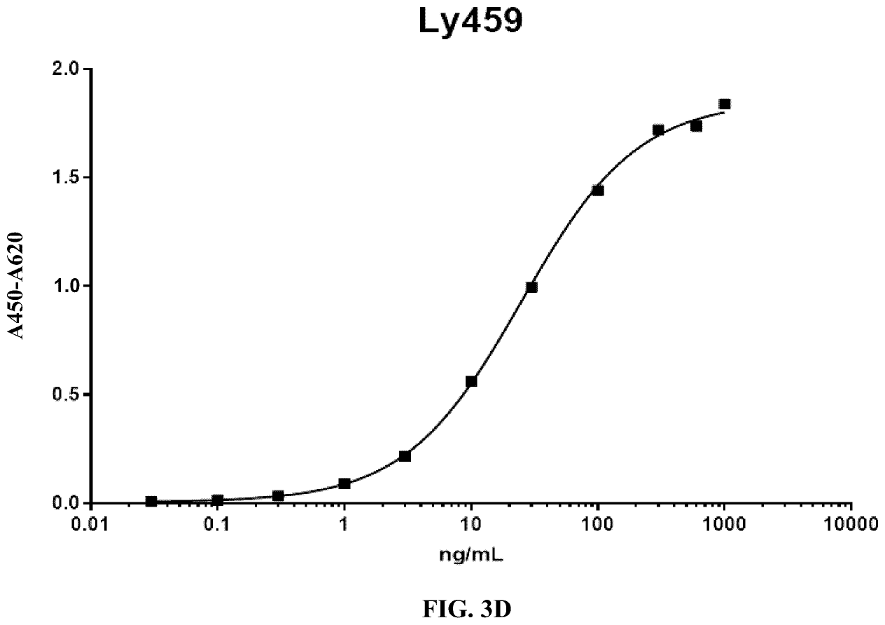

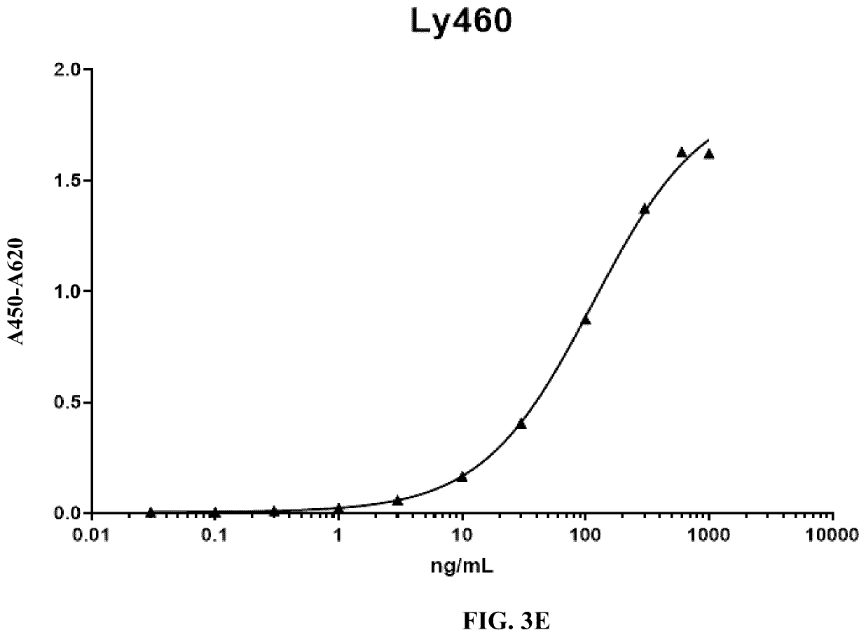

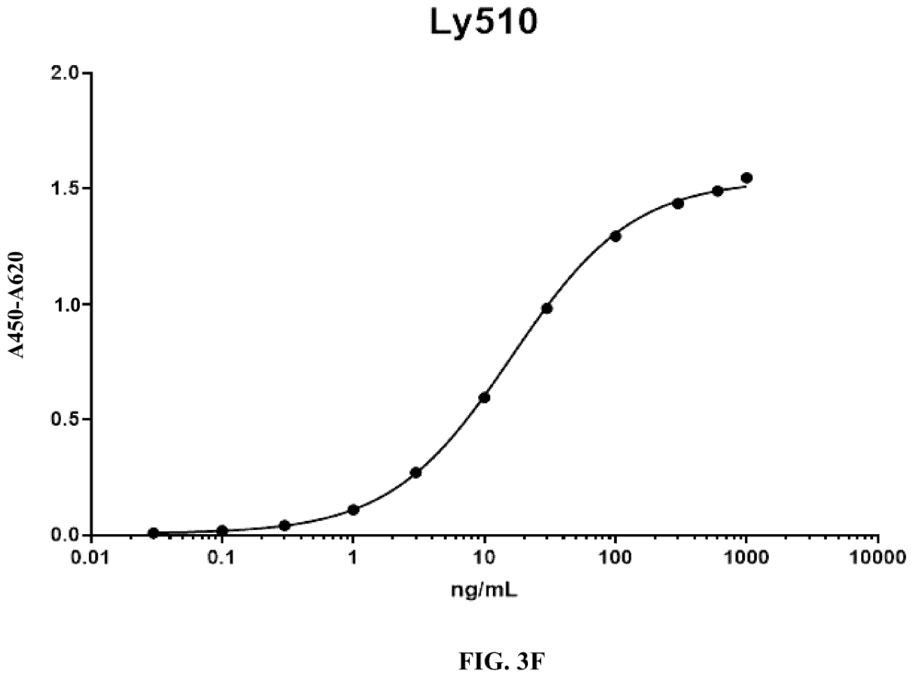

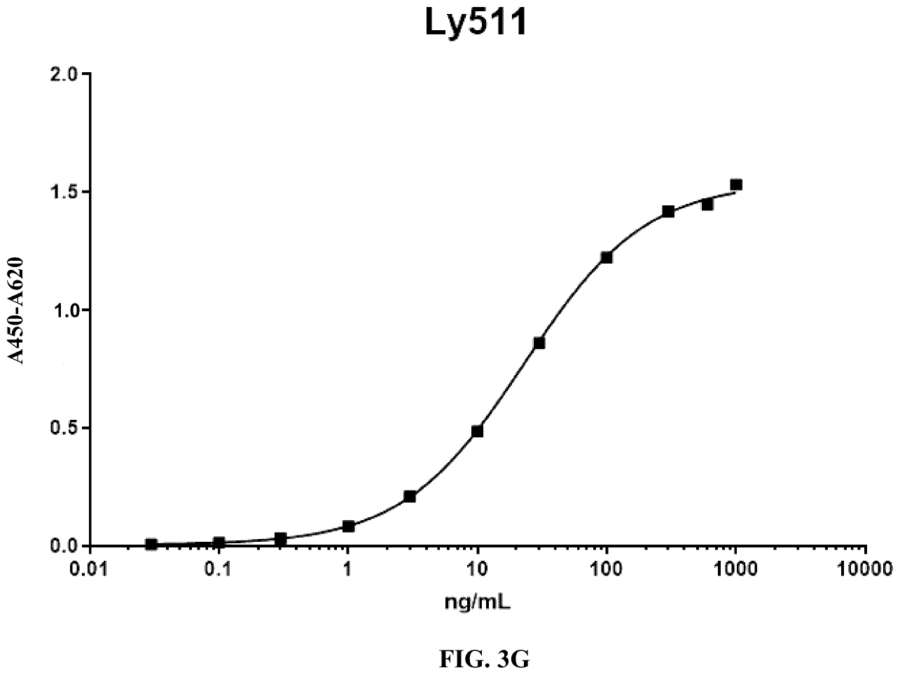

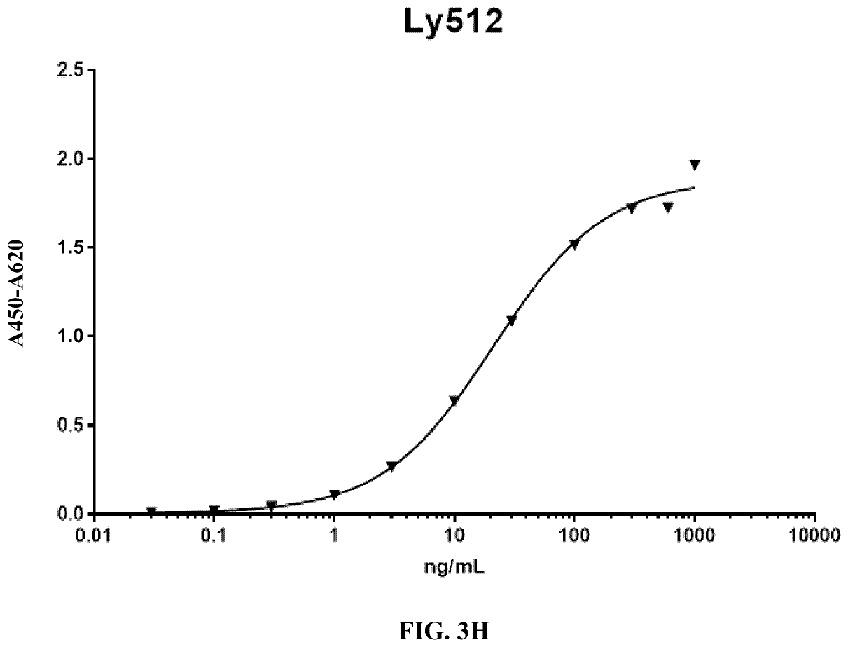

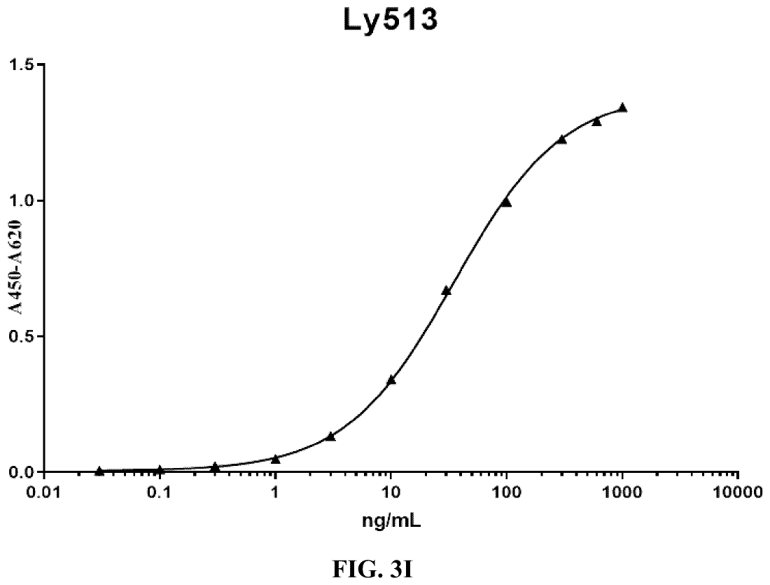

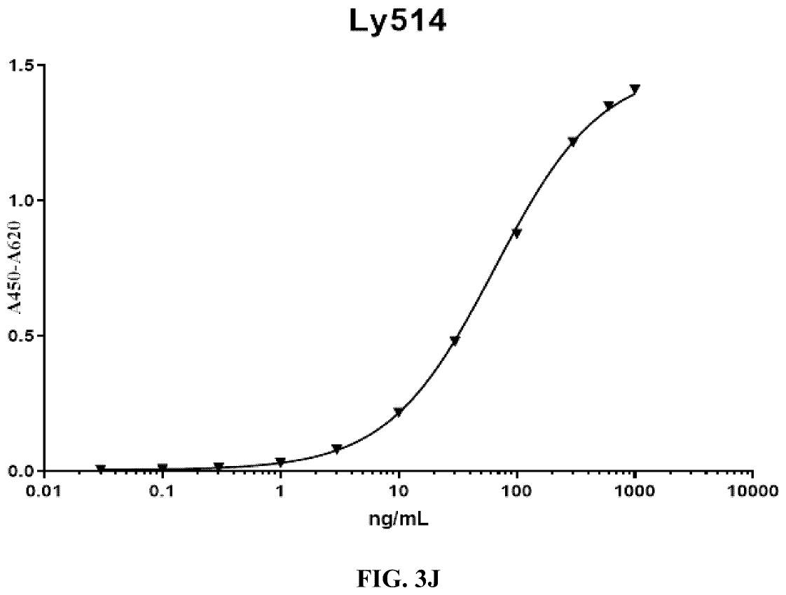

As shown in FIGS. 3A-3J, the exemplary anti-PD-1/CD137 bi-specific antibodies simultaneously binded to recombinant human CD137 and human PD-1 with apparent high affinity.

(ii) Agonistic Activity for CD137

To determine the agonist activity of these anti-PD-1/CD137 bi-specific antibodies, a CD137 reporter assay was developed, which involves reporter cells expressing human CD137 and downstream signaling for IL8 expression. GS-H2-huCD137 reporter cells and PD-1-expressing CHO cells were seeded in the assay plate at 3000 cells/well and 25000 cells/well respectively. Exemplary bi-specific antibodies were added to the assay plate. The assay plate was incubated in 37° C., 5% CO₂ incubator for 18-20 hours. After the 18-20 hour incubation, 8 μL of the supernatant from each well of the assay plate was collected and added to HTRF detection assay plate (Nunc). A Human Interleukin 8 (reporter of CD137 activation) detection assay was performed using a Human IL-8 Assay Kit (Cisbio, Cat #62IL8PEB). In particular, 16 μL assay volume was used. The results were read using Time Resolved Fluorescence by Tecan F200pro and the relative light unit data was recorded.

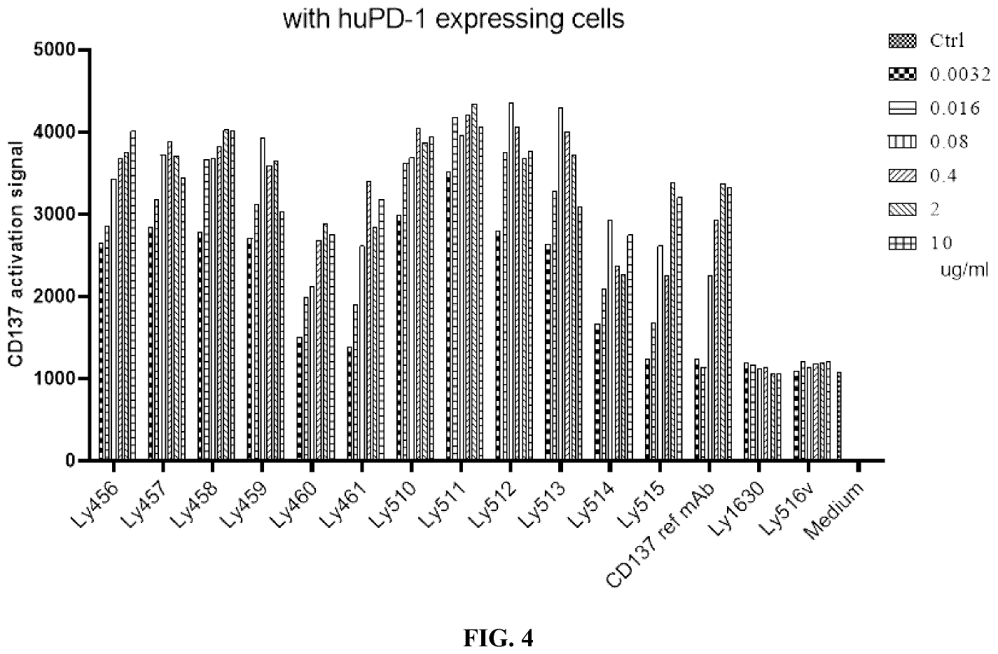

As shown in FIG. 4, the bi-specific antibodies in solution showed a various degree of CD137 agonist activity. A CD137 mAb with known strong agonistic activity (urelumab, WHO INN 9365) was used as a reference (CD137 ref mAb). The CD137 agonist activity was greatly enhanced in the co-culture assay for all the bi-specific antibodies, in particular Ly456, Ly457, Ly458, Ly459, Ly510, Ly511, Ly512 and Ly513. Binding to both CD137 and PD-1 by the tested exemplary bi-specific antibodies simultaneously in a microenvironment would affect individual binding due to, for example, avidity effects, which refer to the accumulated strength of multiple affinities of individual non-covalent binding interactions. The bi-specific antibodies showed increased activity when co-cultured with PD-1 expressing cells.

(iii) Blockage of PD-1/PD-L1 Interaction

To determine the ability of the antibodies in blocking PD-L1/PD-1 cellular function, a reporter assay system was used. The assay consisted of two genetically engineered cell lines: Raji-PD-L1 cells (Raji cells expressing human PD-L1) and Jurkat/NFκB-Luci/PD-1 cells (Jurkat cells expressing human PD-1 and a luciferase reporter driven by an NFkB response element). Briefly, Raji-PD-L1 cells, Jurkat/NFκB-Luci/PD-1 cells and CD137 expressing CHO cells were harvested and aliquoted at 50000 cells/well into a 96-well plate respectively. Then anti-CD3 antibody (1 μg/mL, final concentration) and exemplary bispecific antibodies were added into the 96-well plate. The plate was incubated for additional 6 hours at 37° C. then subjected for Bright-Glo™ Luciferase Assay using Kit from Promega #E2620. Addition of either an anti-PD-1 or anti-PD-L1 antibody that blocks the PD-1/PD-L1 interaction releases the inhibitory signal and results in NFκB-mediated luminescence.

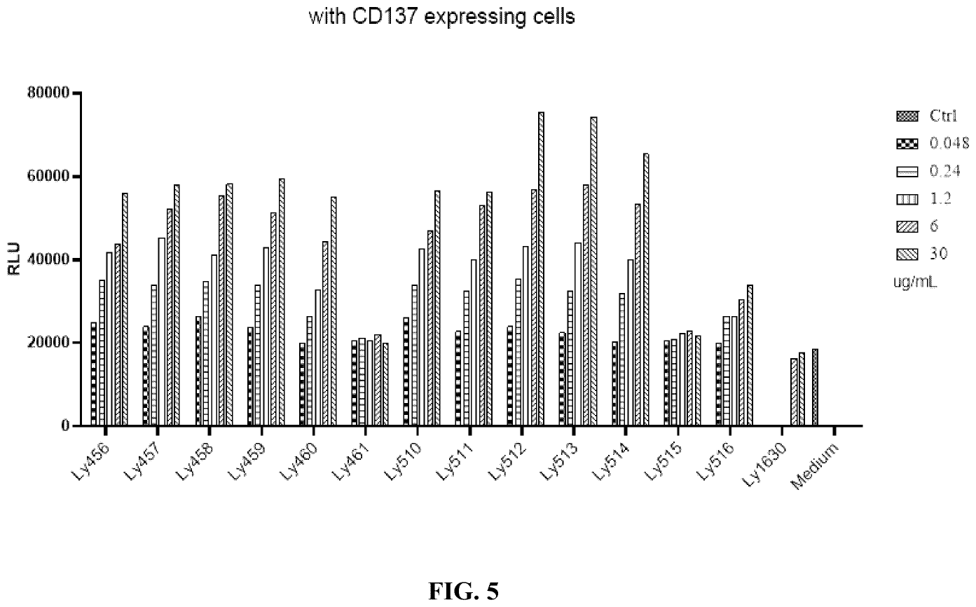

As shown in FIG. 5, the blocking activity was greatly enhanced in the co-culture assay. Binding to both CD137 and PD-1 by the tested bi-specific antibodies simultaneously in a microenvironment would affect individual binding due to at least the avidity effect. The bi-specific antibodies showed increased activity when co-cultured with CD137 expressing cells. Therefore, binding profile to human PD-1 and CD137 would affect the blocking activity of these bi-specific antibodies, with Ly456, Ly457, Ly458, Ly459, Ly460, Ly510, Ly511, Ly512, Ly513 and Ly514 showing more potent and stronger blocking activity.

(iv) Co-Stimulation Activity

Immune cells activation assay are performed to show the co-stimulation functionality of the bispecific antibodies.

(v) PBMC Activation

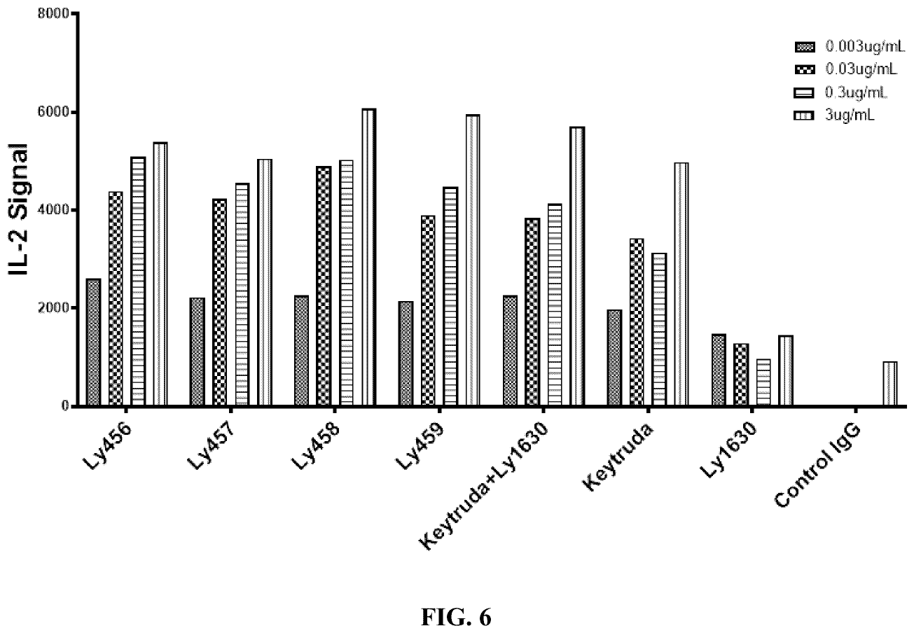

A PBMC activation assay was performed to show the co-stimulation functionality of exemplary bispecific antibodies. Briefly, 2×10⁵ PBMCs in culture medium containing SEB (final concentration at 0.01 μg/mL) were mixed with serial diluted antibody samples. The mixture was placed in plates and incubated at 37° C. with 5% CO₂ for 5 days. Cell culture supernatants were then collected for cytokine detection using a human IL-2 detection kit following the instruction manual. As shown in FIG. 6, the exemplary bispecific antibodies as indicated induced stronger IL-2 production by human PBMCs than Keytruda®, and anti-CD137 mAbs alone or in combination with Keytruda®. Therefore, binding to CD137 and PD-1 by the bi-specific antibodies simultaneously in a microenvironment induced higher levels of PBMC activation as demonstrated by IL-2 secretion, as compared with their parental mAbs, either taken alone or in combination.

(vi) Pharmacokinetic Studies of Anti-PD-1/CD137 Bi-Specific Antibodies

C57BL/6 mice (6-7 weeks old, 19-20 g, female, purchased from Vital River) were used for the study. Antibodies were formulated in DPBS and administered via tail vein injection at 5 mg/kg in a group of 4 mice.

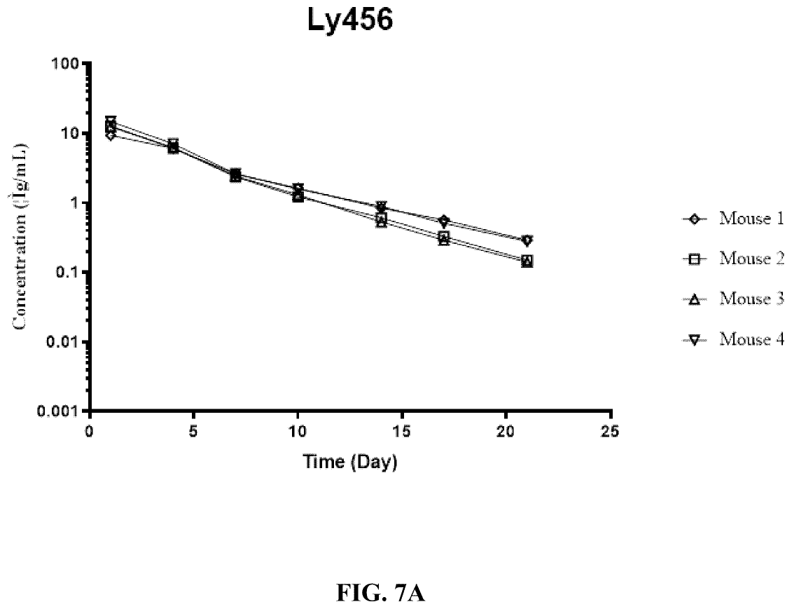

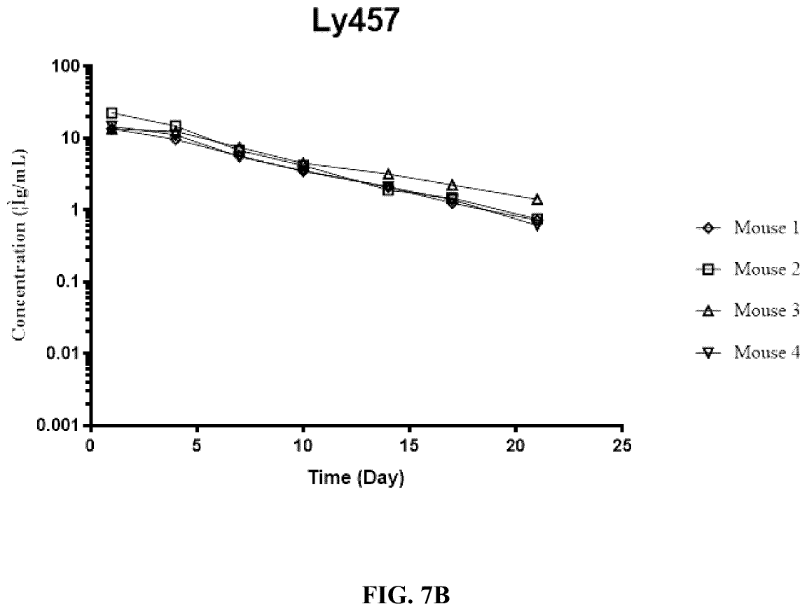

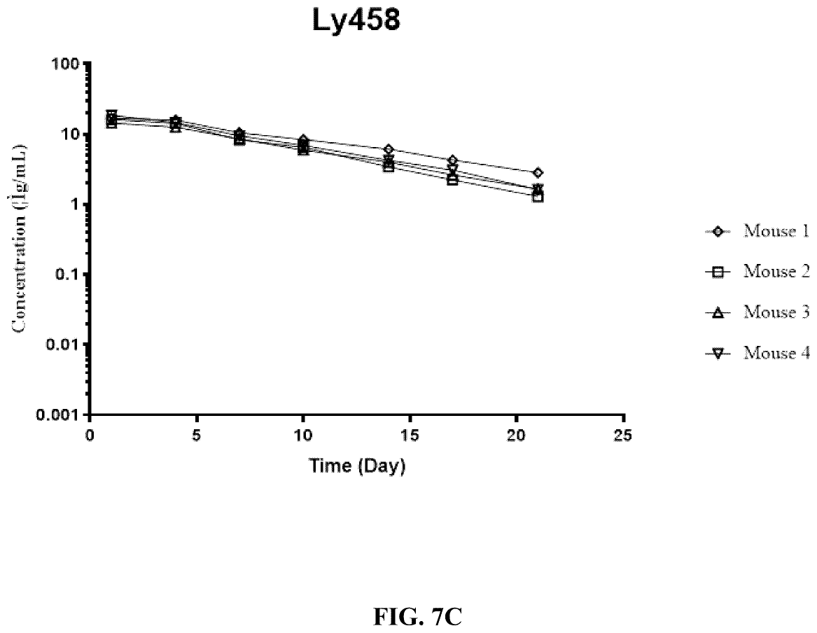

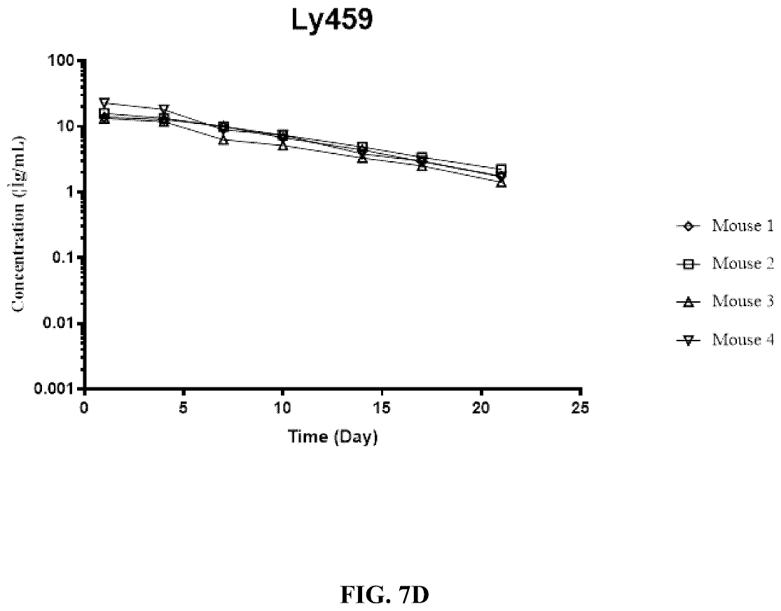

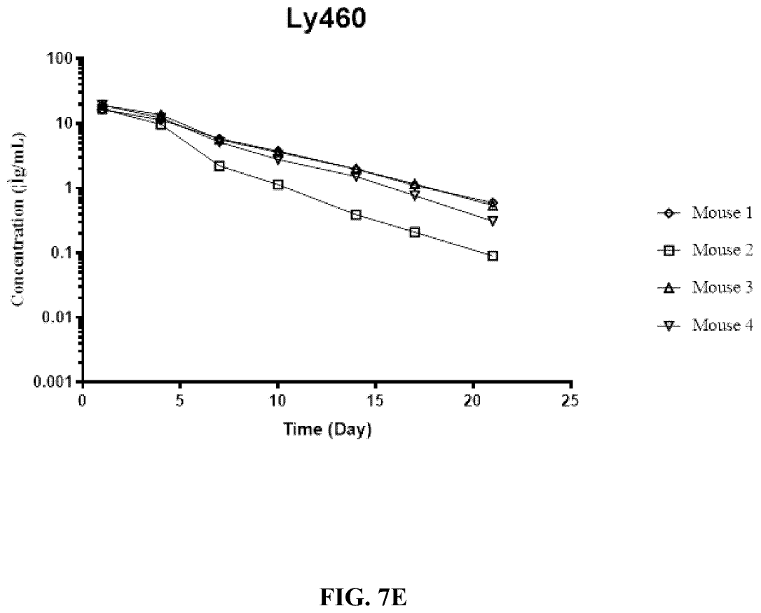

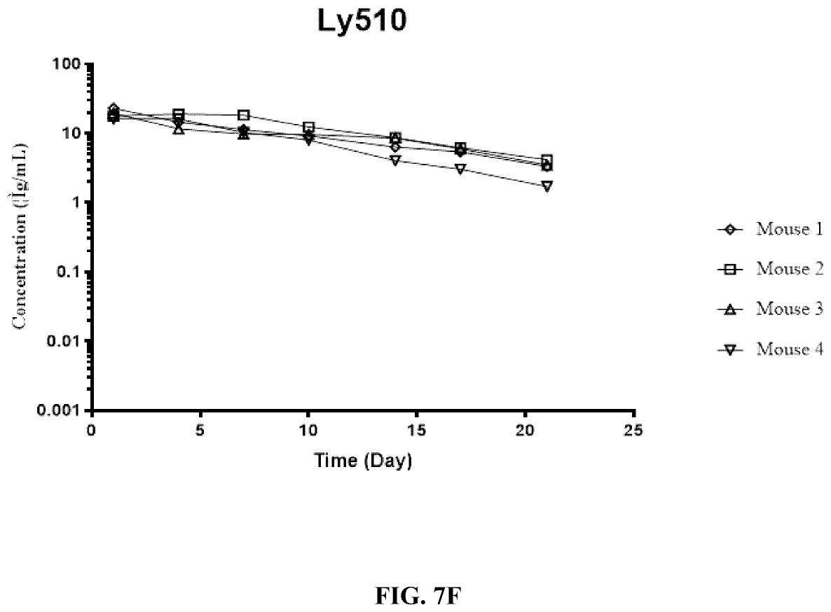

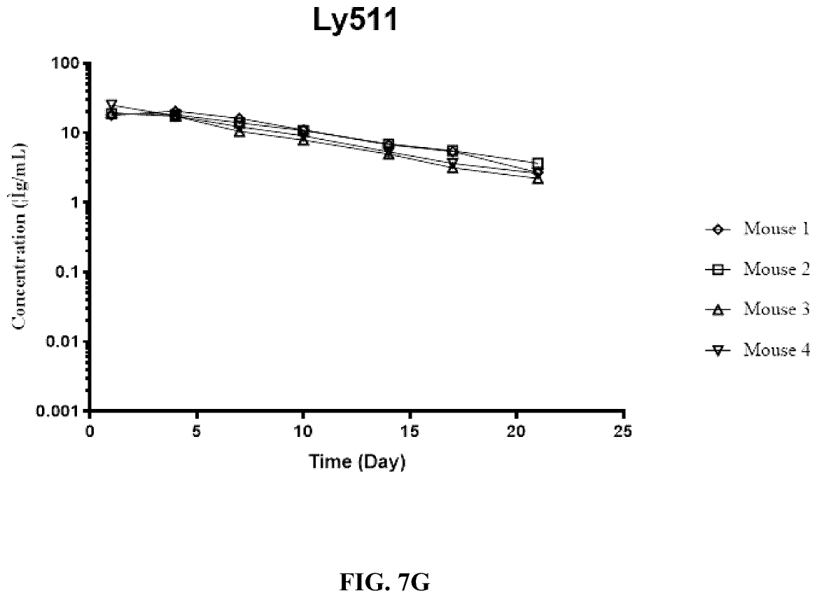

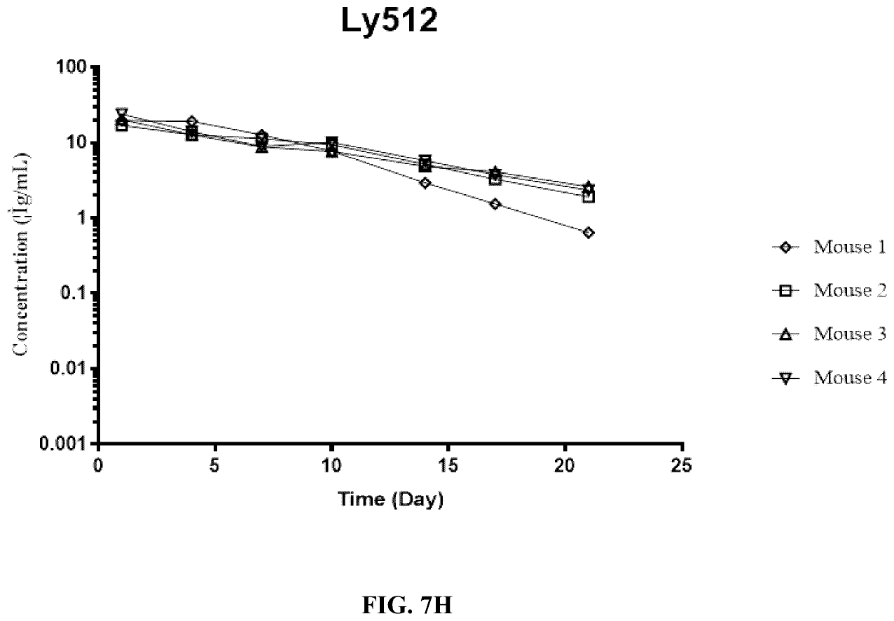

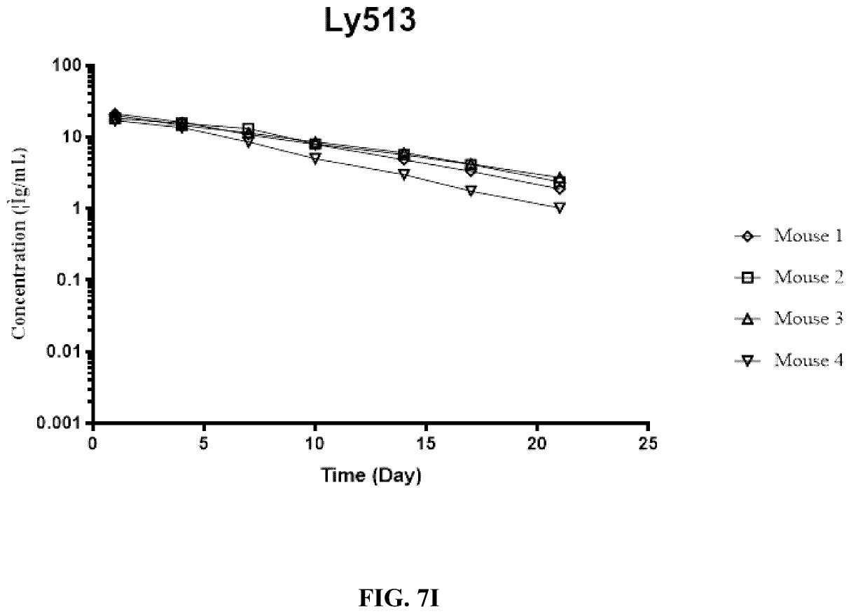

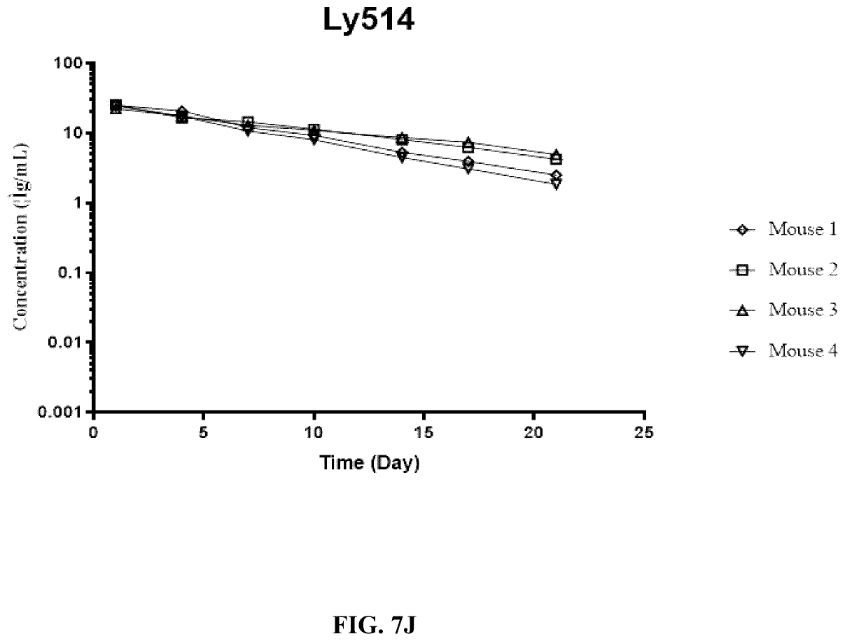

Blood sampling was done at pre-dose, Id, 4d, 7d, 10d, 14d, 17d and 21d by serial bleeding. 10 uL blood per time point was added to 40 uL of a PBS-BSA solution. The sample was then mixed well and centrifuged at 2000 g for 5 minutes at 4° C. The supernatant was put on dry ice immediately after collection and stored at approximately −70° C. until analysis. Blood antibody concentrations were determined by ELISA for their simultaneous binding to recombinant human PD-1 and human CD137, similar to the method described above. FIGS. 7A-7J showed the blood concentrations of the bispecific antibodies after a single intravenous injection of 5 mg/kg. These bispecific antibodies showed high and lasting circulation concentrations.

(vii) Anti-Tumor Activity

Exemplary anti-PD-1/CD137 antibodies were tested in mouse syngeneic tumor models in vivo to determine the anti-tumor efficacy and toxicity of these antibodies. Murine colon cancer MC38-hPD-L1 or B16-OVA tumor cells were subcutaneously implanted into homozygous human CD137/PD-1 double knock-in C57BL/6 mice. Mice were grouped when the tumor size was approximately 150±50 mm³ (n=6). Anti-PD-1/CD137 antibodies were administered by intraperitoneal injections and tumor sizes were measure during 4-6 weeks of antibody treatment. Tumor sizes were calculated as tumor volume using formula of 0.5×length×width²

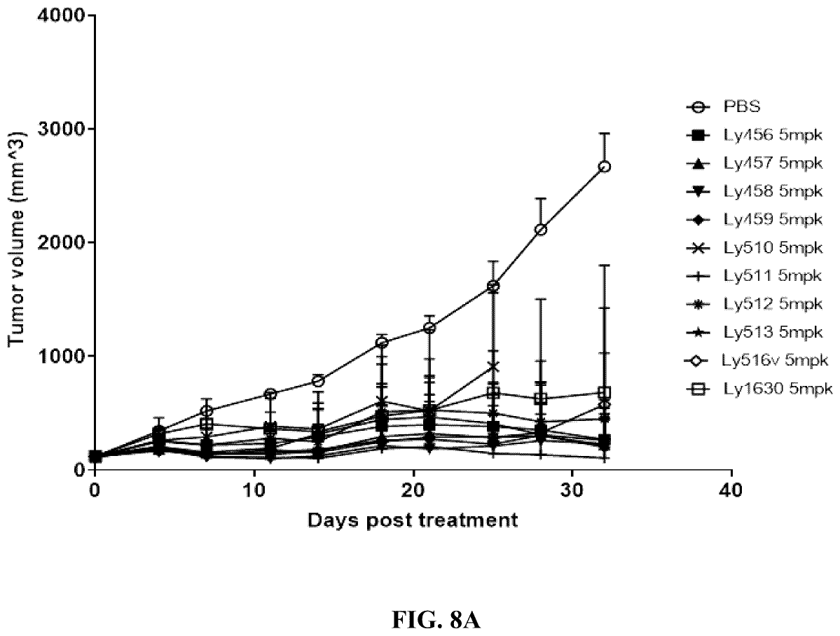

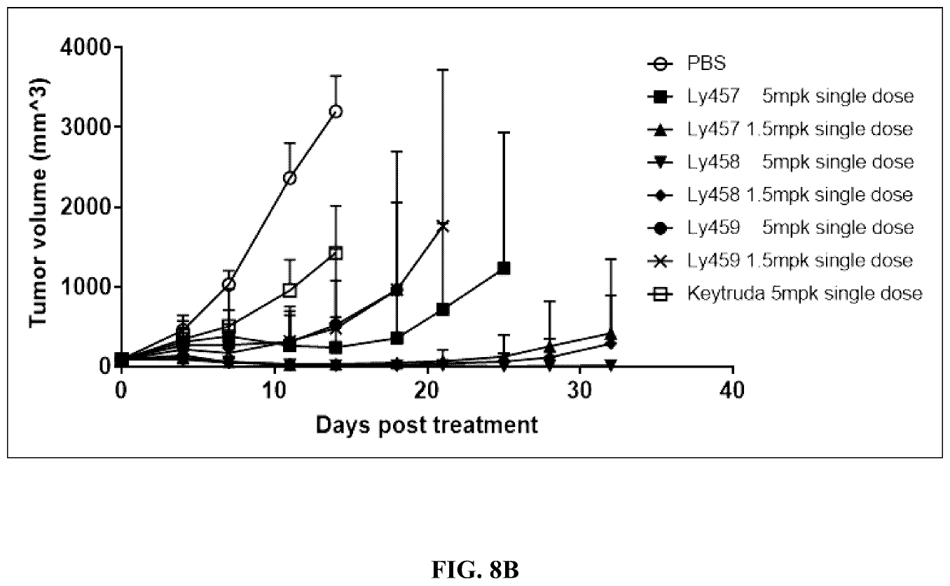

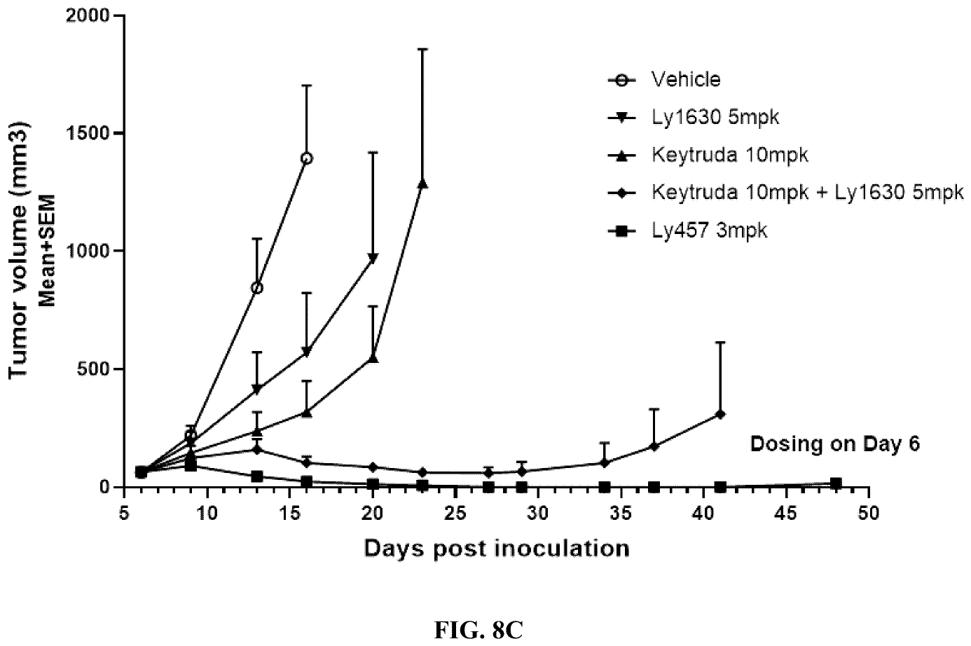

Anti-tumor efficacy was evaluated between tumor sizes of the control group and antibody treatment group as shown in FIGS. 8A-8C. Ly1630 or Ly516v parent antibodies were used as reference controls. Exemplary bispecific antibodies showed comparable or stronger efficacy relative to the parental antibodies in the MC38 model. In the B16-OVA model, Ly457, Ly458 and Ly459 exhibited stronger inhibition of tumor growth than PD-1 antibody Keytruda, either alone or in combination with the parent anti-CD137 antibody

### Example 2: Anti-PD-L1/CD137 Bi-Specific Antibodies

Anti-PD-L1/CD137 bi-specific antibodies were produced using the parent anti-CD137 antibody Ly1630 and anti-PD-L1 antibody Ly076, both of which are humanized antibodies. The V_{H} and V_{L} sequences of Ly1630 are provided in Example 1 above and those of Ly076 are provided below (CDRs determined pursuant to the Kabat scheme are in boldface)

Ly076
V_{L} (SEQ ID NO: 54):
DIQMTQSPSSLSASVGDRVTITCKASQNVYKKLEWYQQKPGKVPKVLIYH
TNILQTGVPSRFSGSGSGTDYTLTISSLQPEDVATYYCYQWNSGPTFGGG
TKVEIK
V_{H} (SEQ ID NO: 55):
QVQLVQSGAEVKKPGASVKVSCKASGYTFTDFWMSWVRQAPGQGLEWMGQ
IYPNTGTTHSNEKFKGRVTMTRDTSTSTVYMELSSLRSEDTAVYYCARSY
HISTTPNWFAYWGQGTLVTVSS

cDNAs encoding the V_{H} and V_{L} chains of both of the parent antibodies were used as the starting materials for constructing anti-CD137/PD-L1 bispecific antibodies. CHO-cell transient expression was carried out with plasmids configured for expressing polypeptide chains of the bi-specific antibodies. These antibodies were purified by protein A affinity chromatography. The amino acid sequences of the multiple-chains of exemplary anti-CD137/PD-L1 bispecific antibodies are provided below:

Ly299
First polypeptide (SEQ ID NO: 56):
QVQLVQSGAEVKKPGASVKVSCKASGYTFTDFWMSWVRQAPGQGLEWMGQIYPNTGTTHSNEKFKGRVT
MTRDTSTSTVYMELSSLRSEDTAVYYCARSYHISTTPNWFAYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGGGGGSGGGGSGGGGSGGGGSDIQMTQSPSSLSA
SVGDRVTITCRASQDIRSNLNWYQQKPGGAVKLLIYYTSRLHSGVPSRFSGSGSGTDYTLTISSLQPED
FATYFCQQSEKLPRTFGGGTKVEIRGGGGSGGGGSGGGGSGGGGSQVQLVQSGAEVKKPGASVKVSCKA
SGYTFAGFEMHWVRQAPGQGLEWMGAIDPKTGGTDYNQKFKDRVTMTRDTSISTAYMELSRLRSDDTAV
YYCARDLGYFDVWGQGTLVTVSS
Second polypeptide: (SEQ ID NO: 57)
DIQMTQSPSSLSASVGDRVTITCKASQNVYKKLEWYQQKPGKVPKVLIYHTNILQTGVPSRFSGSGSGT
DYTLTISSLQPEDVATYYCYQWNSGPTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGEC
Ly346
First polypeptide (SEQ ID NO: 58):
QVQLVQSGAEVKKPGASVKVSCKASGYTFTDFWMSWVRQAPGQGLEWMGQIYPNTGTTHSNEKFKGRVT
MTRDTSTSTVYMELSSLRSEDTAVYYCARSYHISTTPNWFAYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGGGGGSGGGGSGGGGSGGGGSQVQLVQSGAEVKK
PGASVKVSCKASGYTFAGFEMHWVRQAPGQGLEWMGAIDPKTGGTDYNQKFKDRVTMTRDTSISTAYME
LSRLRSDDTAVYYCARDLGYFDVWGQGTLVTVSSGGGGSGGGGSGGGGSGGGGSDIQMTQSPSSLSASV
GDRVTITCRASQDIRSNLNWYQQKPGGAVKLLIYYTSRLHSGVPSRFSGSGSGTDYTLTISSLQPEDFA
TYFCQQSEKLPRTFGGGTKVEIR
Second polypeptide: (SEQ ID NO: 57):
same as the second polypeptide of Ly299
Ly347
First polypeptide: (SEQ ID NO: 59):
QVQLVQSGAEVKKPGASVKVSCKASGYTFTDFWMSWVRQAPGQGLEWMGQIYPNTGTTHSNEKFKGRVT
MTRDTSTSTVYMELSSLRSEDTAVYYCARSYHISTTPNWFAYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPG
Second polypeptide (SEQ ID NO: 60):
DIQMTQSPSSLSASVGDRVTITCKASQNVYKKLEWYQQKPGKVPKVLIYHTNILQTGVPSRFSGSGSGT
DYTLTISSLQPEDVATYYCYQWNSGPTFGGGTKVEIKRTVAAPSVFIFPPSDEQLKSGTASVVCLLNNE
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSQVQLVQSGAEVKKPGASVKVSCKASGYTFAGFEMHWVRQAPGQ
GLEWMGAIDPKTGGTDYNQKFKDRVTMTRDTSISTAYMELSRLRSDDTAVYYCARDLGYFDVWGQGTLV
TVSSGGGGSGGGGSGGGGSGGGGSDIQMTQSPSSLSASVGDRVTITCRASQDIRSNLNWYQQKPGGAVK
LLIYYTSRLHSGVPSRFSGSGSGTDYTLTISSLQPEDFATYFCQQSEKLPRTFGGGTKVEIR
Ly348
First polypeptide: (SEQ ID NO: 59):
same first polypeptide as Ly347
Second polypeptide (SEQ ID NO: 61):
DIQMTQSPSSLSASVGDRVTITCKASQNVYKKLEWYQQKPGKVPKVLIYHTNILQTGVPSRFSGSGSGT
DYTLTISSLQPEDVATYYCYQWNSGPTFGGGTKVEIKRTVAAPSVFIFPPSDEQLKSGTASVVCLLNNE
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSDIQMTQSPSSLSASVGDRVTITCRASQDIRSNLNWYQQKPGGA
VKLLIYYTSRLHSGVPSRFSGSGSGTDYTLTISSLQPEDFATYFCQQSEKLPRTFGGGTKVEIRGGGGS
GGGGSGGGGSGGGGSQVQLVQSGAEVKKPGASVKVSCKASGYTFAGFEMHWVRQAPGQGLEWMGAIDPK
TGGTDYNQKFKDRVTMTRDTSISTAYMELSRLRSDDTAVYYCARDLGYFDVWGQGTLVTVSS

### Characterization of Anti-PD-L1/CD137 Bi-Specific Antibodies

(i) Binding Activity

Exemplary anti-PD-L1/CD137 bi-specific antibodies were analyzed by FACS for their binding properties to human PD-L1 or human CD137 expressed on CHO cells, following the procedures disclosed in Examples 1 above.

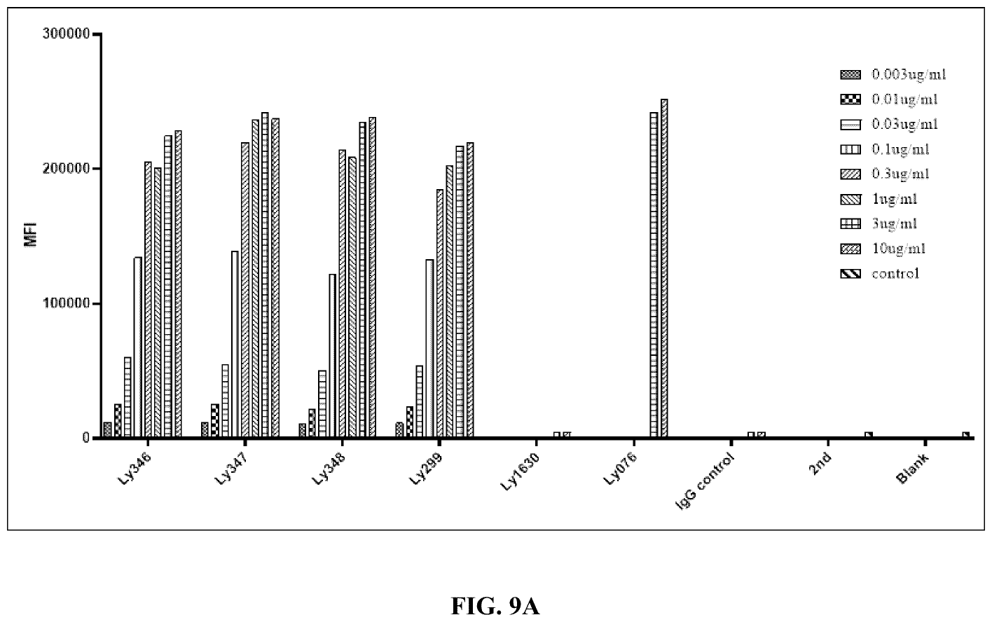

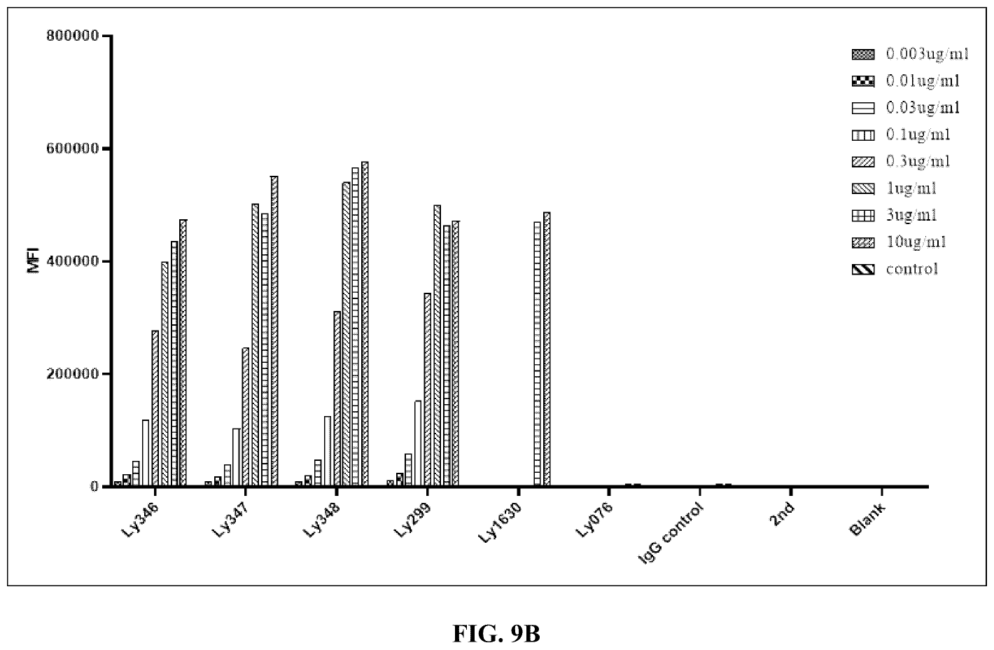

As shown in FIG. 9A, the tested exemplary anti-PD-L1/CD137 bi-specific antibodies exhibited similar binding affinity to human PD-L1 expressed on the CHO cells relative to Ly076. As shown in FIG. 9B, the bi-specific antibodies exhibited similar binding affinity to human CD137 expressed on CHO cells relative to Ly1630. Compared to the corresponding parental antibodies, the binding activity of bi-specific antibodies are substantially the same.

Exemplary anti-PD-L1/CD137 bi-specific antibodies were analyzed by ELISA for their simultaneous binding to recombinant human PD-L1 and human CD137. Briefly, the ECD portion of human CD137 protein (mouse IgG2a Fc tag) was diluted and coated onto an ELISA plate with a volume of 100 μL/well by incubation at 4° C. overnight. The next day, the plate was blocked with PBST-BSA buffer, then serially diluted samples of anti-PD-L1/CD137 bi-specific antibodies were pipetted into appropriate wells at 50 μL/well, and the plate was incubated for 1 h followed by Washing. Human PD-L1 ECD fragment (His tag) was added into the plate at 50 μL/well. After 1-hour incubation at room temperature, HRP-conjugated anti-His tag antibody was added into the plate at 100 μL/well and followed by anding HRP-conjugated secondary antibody. The plate was incubated for 1 hour at room temperature followed by washing. TMB substrate solution was added at 100 μL/well and the color development was stopped by adding 100 uL/well Stop Solution (2N H₂SO₄). Absorbance at 450 nm and 620 nm was read by Tecan F200 Pro plate reader. GraphPad 7.0, “[Agonist] vs. response—Variable slope (four parameters)” was used to plot the binding data and calculate binding EC50 values.

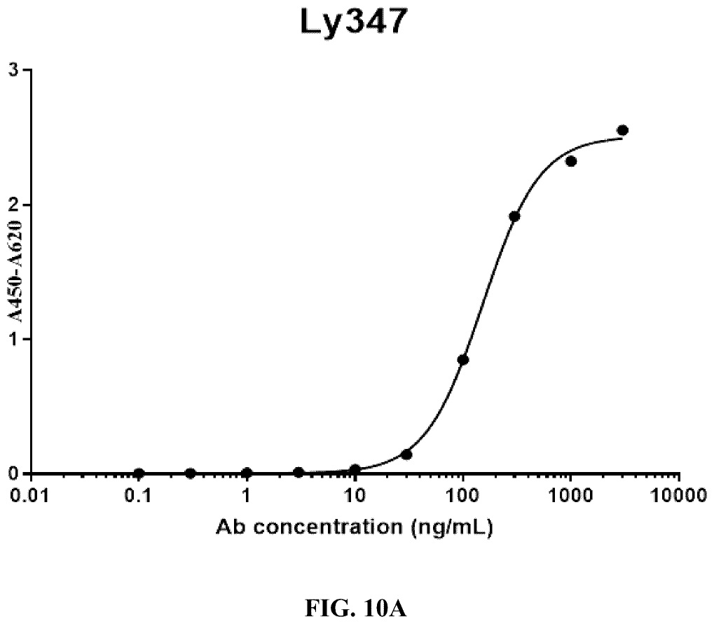

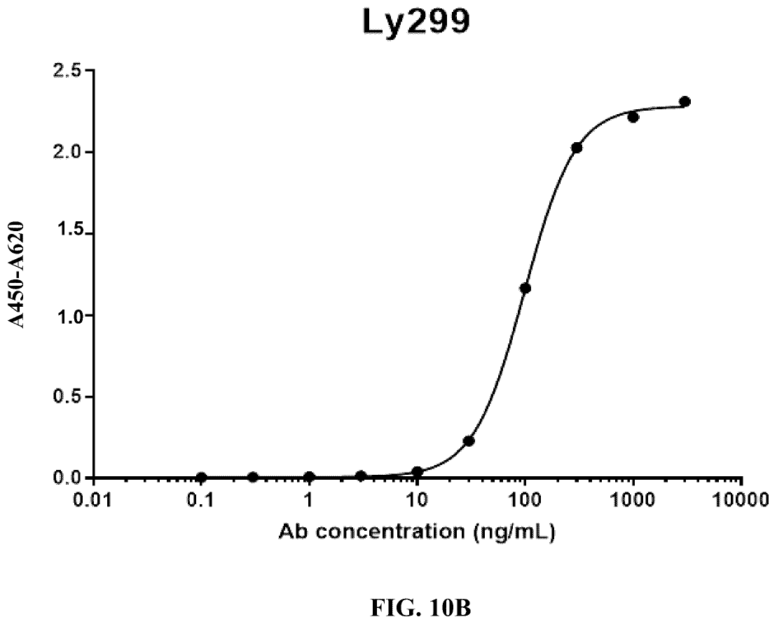

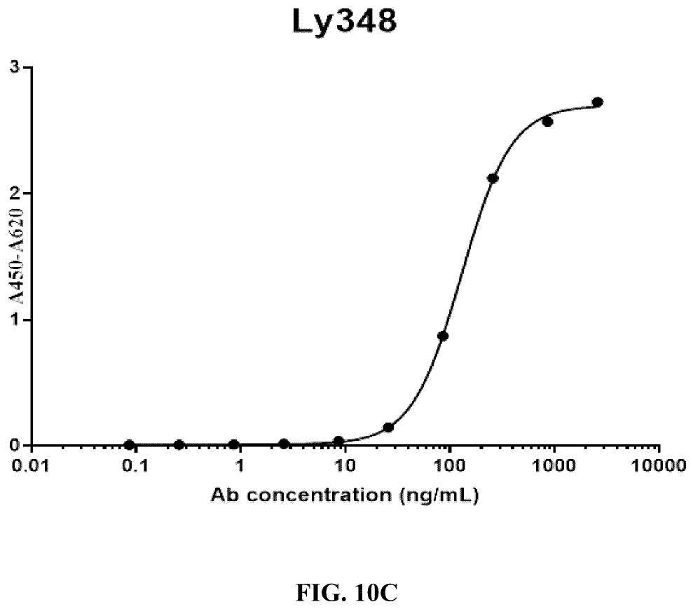

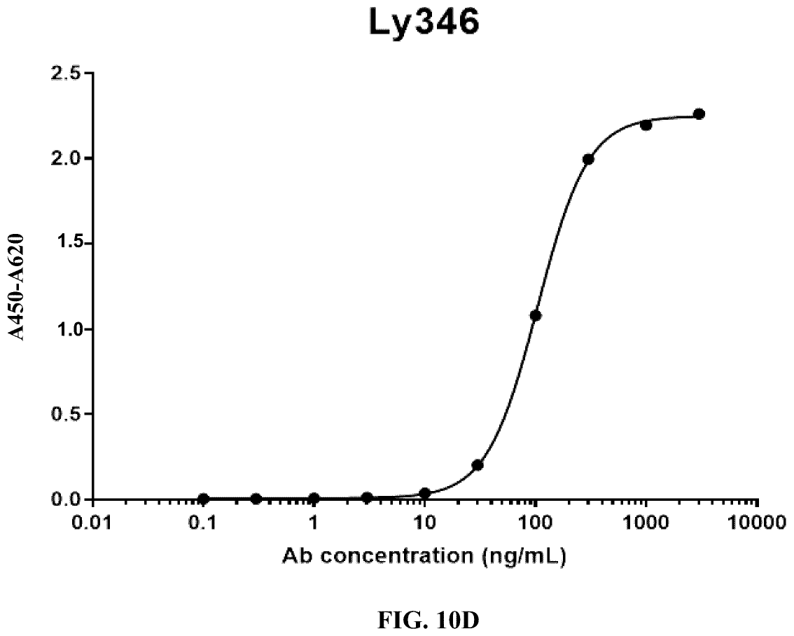

As shown in FIGS. 10A-10D, the exemplary anti-PD-L1/CD137 bi-specific antibodies simultaneously binded to recombinant human CD137 and human PD-L1 with apparent high affinities.

(ii) Agonistic Activity for CD137

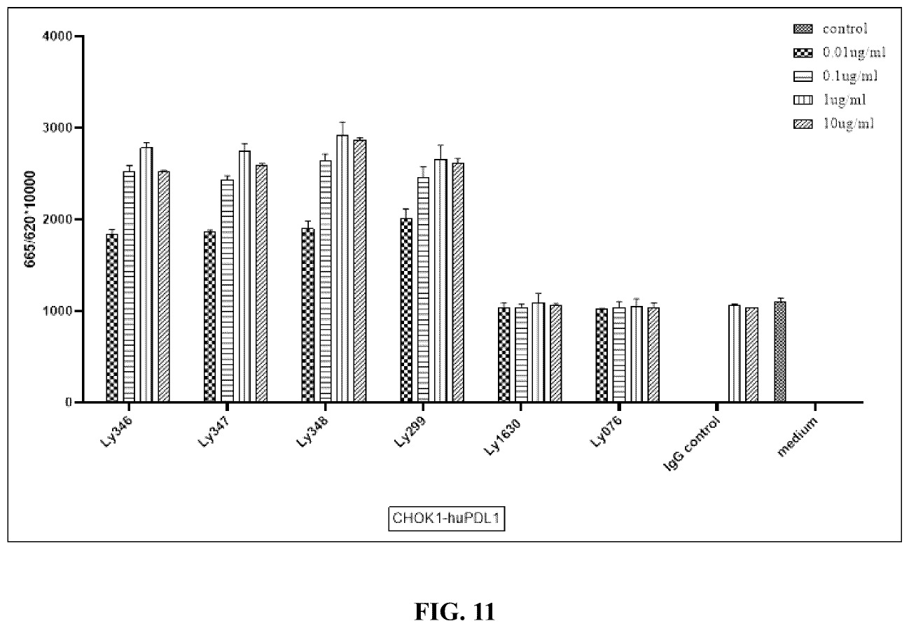

The CD137 reporter assay disclosed herein was used to determine the agonist activity of the bispecific antibodies, following the same procedures disclosed in Example 1 above. The CD137 reporter assay was performed in co-culture with PD-L1-expressing CHO cells. As shown in FIG. 11, the bi-specific antibodies showed agonist activity when co-cultured with PD-L1-expressing CHO cells, while CD137 parental antibody Ly1630 didn't, suggesting that the bispecific antibodies mediated CD137 stimulation is strictly PD-L1 dependent.

(iii) PBMC Activation

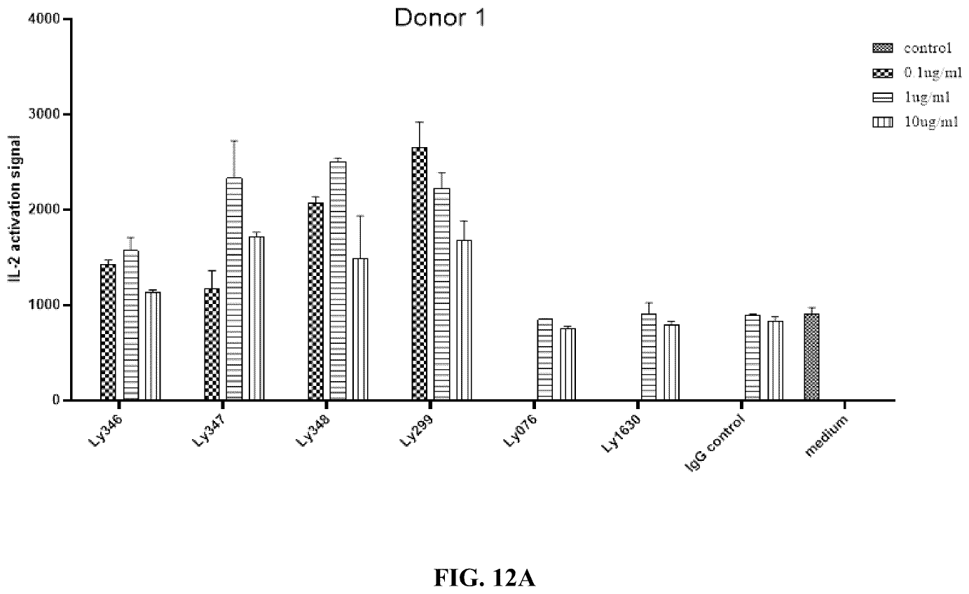

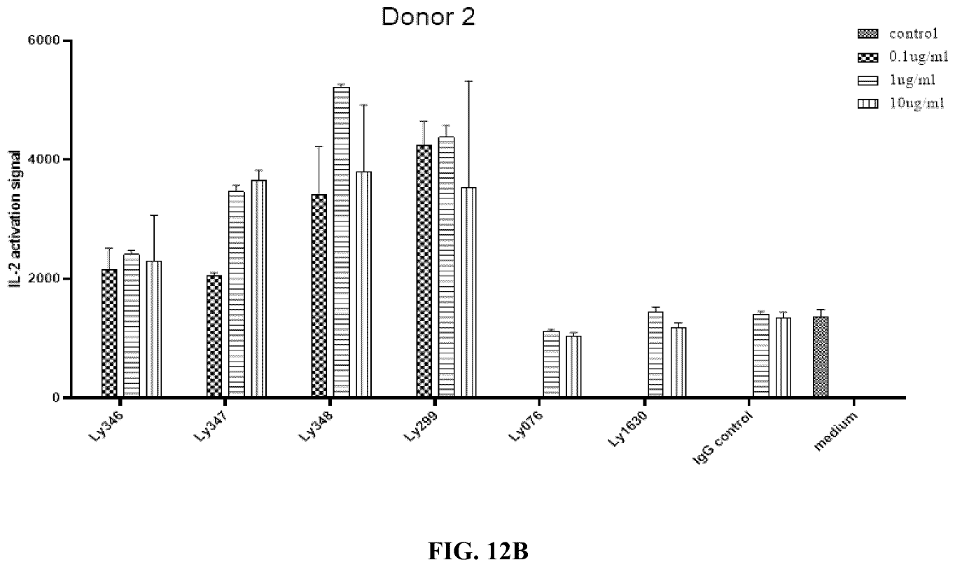

Human PBMC activation assays were performed to show the co-stimulation functionality of the exemplary bispecific antibodies. Briefly, 2×10⁵ PBMCs, 1×10⁴ PD-L1 expressing CHO cells and serial diluted antibody samples were added to plates (pre-coated 2 μg/mL OKT3) and incubated at 37° C. with 5% CO₂ for 3 days. Cell culture supernatants were then collected for cytokine detection using Human IL-2 detection kit (Cisbio) following the instruction manual. FIGS. 12A-12B show that the exemplary bispecific antibodies induced higher IL-2 production from human PBMCs in this assay, as compared to the parental antibodies. Therefore, binding to CD137 and PD-L1 by the bi-specific antibody molecules simultaneously in a microenvironment enhanced PBMC co-stimulation activity.

(iv) Pharmacokinetic Studies of Anti-PD-L1/CD137 Bi-Specific Antibodies

C57BL/6 mice (6-7 weeks old, 19-20 g, female, purchased from Vital River) were used for the study. Antibodies were formulated in PBS and administered via tail vein injection at 5 mg/kg in a group of 4 mice.

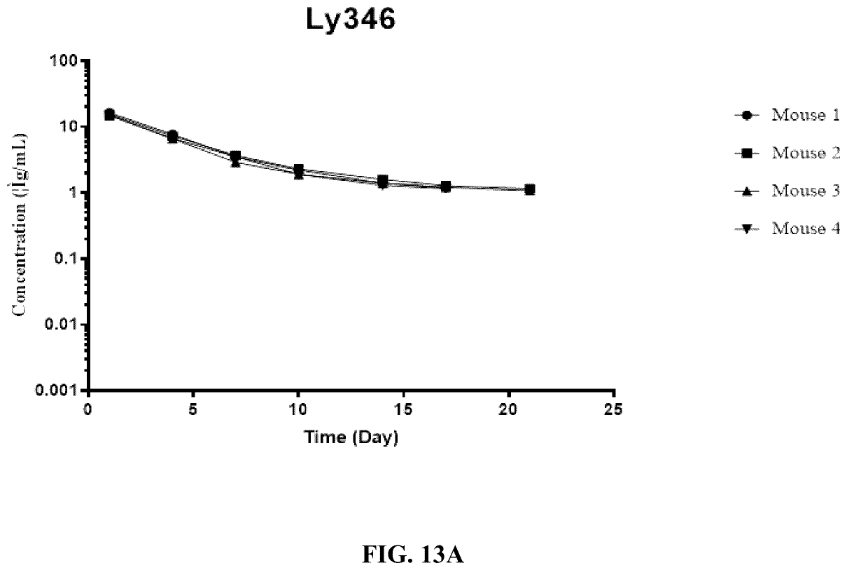

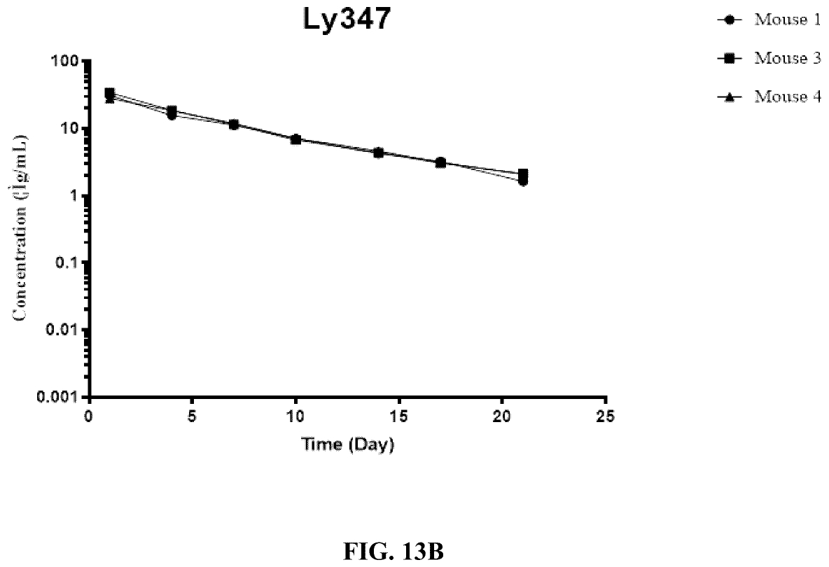

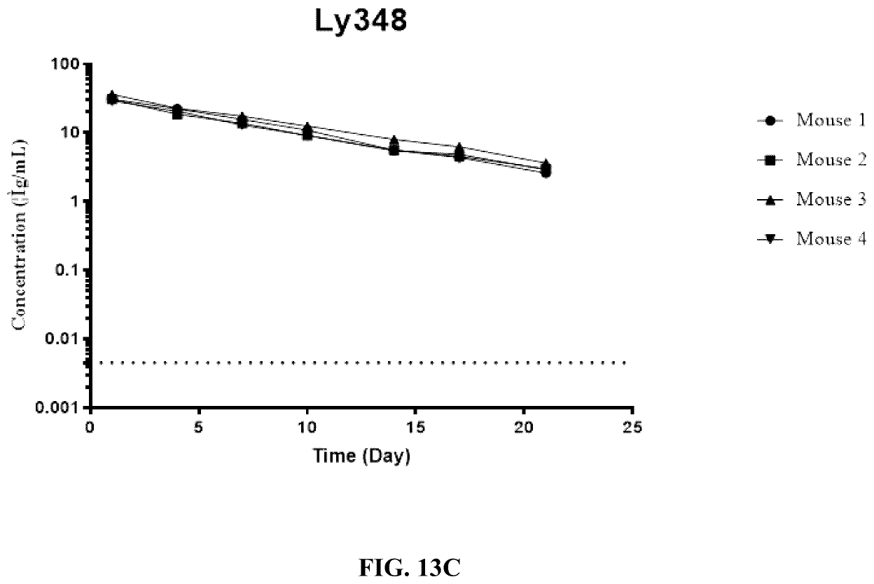

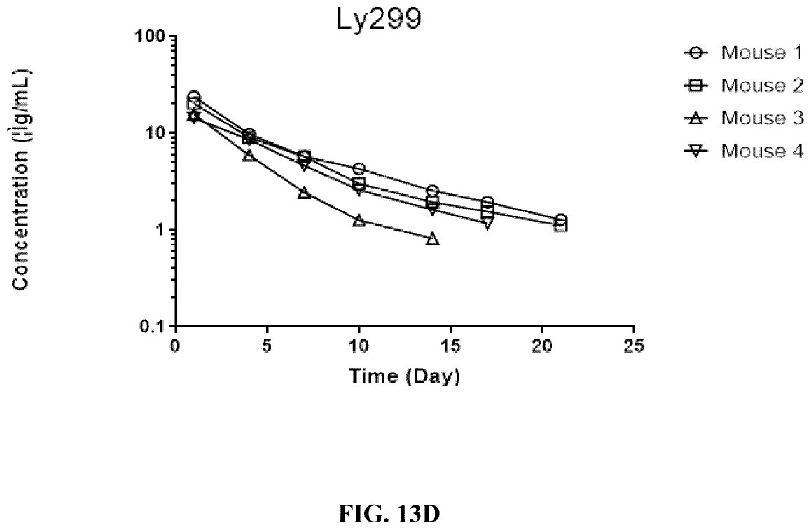

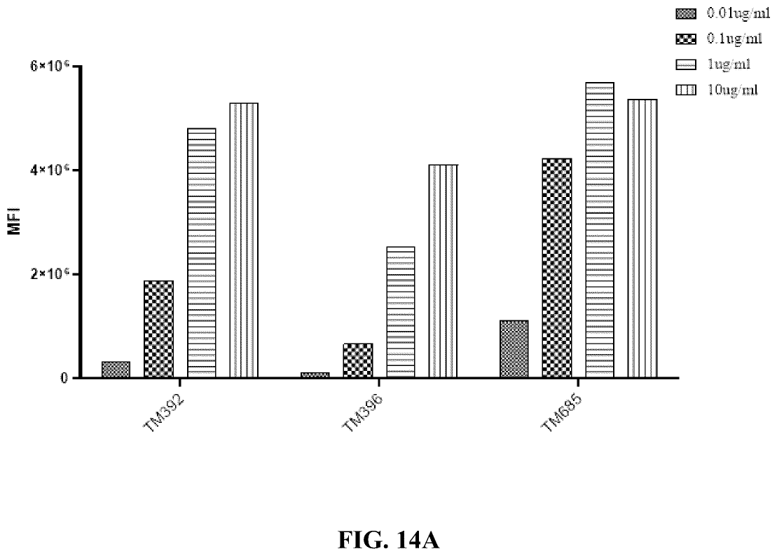

Blood sampling was done at pre-dose, Id, 4d, 7d, 10d, 14d, 17d and 21d by serial bleeding. 10 uL blood per time point was added to 40 uL of a PBS-BSA solution. The sample was then mixed well and centrifuged at 2000 g for 5 minutes at 4° C. The supernatant was put on dry ice immediately after collection and stored at approximately −70° C. until analysis. Blood antibody concentrations were determined by ELISA for simultaneous binding to PD-L1 and CD137 described above. FIGS. 13A-13D showed the blood concentrations of exemplary bispecific antibodies after a single intravenous injection of 5 mg/kg. These bispecific antibodies showed high and lasting circulation concentrations.

### Example 3: Anti-GITR/CD137 Bi-Specific Antibodies

(i) Construction of Anti-GITR Antibodies

Anti-human GITR antibodies were generated using standard murine hybridoma technology. Exemplary anti-GITR antibody, LYV392 and LYV396, were developed. The amino acid sequences of the V_{H} and V_{L} chains of antibody LYV392 and antibody LYV396 were analyzed and the CDRs were identified following the Kabat CDR definitions. The V_{H} and V_{L} sequences of LYV392 and LYV396 are provided below with the CDR regions identified in boldface:

>LYV392_VH
((SEQ ID NO: 62)
QVQLKESGPGLVQPSETLSLTCNVSGFSLTSYNVHWVRQPPGKGLEW
LGVIWSGVRTDYNSVLKSRLSIRWDSSKNQVFLKMNSLQSEDTATYY
CARGTYDANYHDVMDAWGQGASVTVSS
>LYV392_VL
((SEQ ID NO: 63)
DTVLTQSPALAVSPGERVTISCRASKSVRTGMHWYQQKPGQQPKLLV
YGASNLESGVPARFSGSGSGTDFTLTIDPVEADDIATYFCHQSWNHF
TFGSGTKLEIKR
>LYV396_VH
(SEQ ID NO: 64)
QVQLKESGPGLVQPSETLSLTCTVSGFSLTSYNVHWVRQPPGKGLEW
LGVIWSGVRTDYNSVLKPRLSISRDSSKNKVFLNMNSLQSEDTATYY
CARGTYDDNYHDVMDAWGQGASVTVSS
>LYV396_VL
(SEQ ID NO: 65)
DTVLTQSPALAVSPGERVTISCRASKSVRTGMHWYQQKPGQQPKLLI
YGASNLESGVPVRFSGSGSGTDFTLTIDPVEADDTATYFCQQSWNHF
TFGSGTKLEIKR

Humanized Anti-GITR Antibodies Derived from LYV392

Sequence alignments were performed to compare the LYV392 V_{H} and V_{L} to human germline V_{H} and V_{L} sequences, respectively, following methods known in the art. See, e.g., Glanville J. et al. PNAS 2009; 106 (48) 20216-21. Based on overall sequence identity, matching interface positions and similarly classed CDR canonical positions, a germline family was identified for each of the light and heavy chains as the desired acceptor frameworks, i.e., IGKV3-11*01 for the light chain and IGHV4-59*01 for the heavy chain. Human acceptors were identified as ANV21835.1 immunoglobulin kappa light chain and AAS86012.1 immunoglobulin heavy chain variable region, the amino acid sequences of which are shown below:

>AAS86012.1 Human VH acceptor sequence
(SEQ ID NO: 66)
LPDGVLSQVQLQESGPGLVKPSETLSLTCTVSGGSISSYYWSWIRQP
PGKGLEWIGYIYYSGSTNYNPSLKSRVTISVDTSKNQFSLKLSSVTA
ADTAVYYCARDQGYTYGGDAFDVWGQGTMVTVSSASTKGPSVFPLAP
SSKSTSGGTTALGCLVKDYFP
>ANV21835.1 Human VL acceptor sequence
(SEQ ID NO: 67)
MEAPAQLLFLLLLWLPDTTGEIVLTQSPATLSLSPGERATLSCRASQ
SVSSYLAWYQQKPGQAPRLLIYDASNRATGIPARFSGSGSGTDFTLT
ISSLEPEDFAVYYCQQRSNWRTFGQGTKLEIKRTVAAPSVFIFPPSD
EQLKSGTASVVCLLNNFYP

The CDRs of the parent LYV392 antibody were grafted into the corresponding CDR regions of the above-noted human V_{H} and V_{L} acceptor sequences to generate humanized LYV392_VH-1 and LYV392_VL-1 chains (grafted humanized antibody), the amino acid sequence of each of which is provided below (CDRs in boldface):

> LYV392_VH-1 (grafted LYV392_VH; SEQ ID NO: 68)
QVQLQESGPGLVKPSETLSLTCTVSGFSLTSYNVHWIRQPPGKGLEWI
GVIWSGVRTDYNSVLKSRVTISVDTSKNQFSLKLSSVTAADTAVYYCA
RGTYDANYHDVMDAWGQGTMVTVSS
> LYV392_VL-1 (grafted LYV392_VL; SEQ ID NO: 69)
EIVLTQSPATLSLSPGERATLSCRASKSVRTGMHWYQQKPGQAPRLLI
YGASNLESGIPARFSGSGSGTDFTLTISSLEPEDFAVYYCHQSWNHFT
FGQGTKLEIKR

Homology modeling of LYV392 antibody Fv fragments was carried out as follows. Briefly, the LYV392 VH and VL sequences were BLAST searched against the PDB antibody database to identify a suitable template for Fv fragments and especially for building the domain interface. Structural template 1A70 (FV FRAGMENT OF MOUSE MONOCLONAL ANTIBODY D1.3 (BALB/C, IGG1, K) R96L DELETION MUTANT ON VARIANT FOR CHAIN L GLU81->ASP AND CHAIN H LEU312->VAL) was selected, identity=63%. The amino acid sequence alignment between the LYV392 antibody (SEQ ID NO:70) and the 1A70 template (SEQ ID NO:71) is shown below.

| | |
|---|---|
| 1A7O | QVQLQESGPGLVAPSQSLSITCTVSGFSLTGYGVNWVRQPPGKGLEWLGMIWGDGNTDYN |
| LYV392 | QVQLKESGPGLVQPSETLSLTCNVSGFSLTSYNVHWVRQPPGKGLEWLGVIWSGVRTDYN |
| 1A7O | SALKSRLSISKDNSKSQVFLKMNSLHTDDTARYYCARER-----DYRLDYWGQGTTVTVS |
| LYV392 | SVLKSRLSIRWDSSKNQVELKMNSLQSEDTATYYCARGTYDANYHDVMDAWGQGASVTVS |
| 1A7O | SDIVLTQSPASLSASVGETVTITCRASGNIHNYLAWYQQKQGKSPQLLVYYTTTLADGVP |
| LYV392 | SDTVLTQSPA-LAVSPGERVTISCRASKSVRTGMHWYQQKPGQQPKLLVYGASNLESGVP |
| 1A7O | SRFSGSGSGTQYSLKINSLQPDDFGSYYCQHFWSTPTFGGGTKLEIK- |
| LYV392 | ARFSGSGSGTDFTLTIDPVEADDIATYFCHQSWNHFTFGSGTKLEIKR |

Homology models were built using customized Build Homology Models protocol. Disulfide bridges were specified and linked. Loops were optimized using DOPE method. Based on the homology model of 1A70, the V_{H} and V_{L} sequences of the LYV392 antibody were analyzed. Framework region (FR) residues that are expected to be important for the binding activity, including canonical FR residues and V_{H}-V_{L} interface residues of the antibody were identified. The framework residues in the inner core were further analyzed and residues of LYV392_VL-1 (grafted LYV392_VL) were identified for back mutations, including E1D, I2T, I48V, V85T, and Y87F.

>LYV392_VL-2
(SEQ ID NO: 72)
DTVLTQSPATLSLSPGERATLSCRASKSVRTGMHWYQQKPGQAPRLLV
YGASNLESGIPARFSGSGSGTDFTLTISSLEPEDFATYFCHQSWNHFT
FGQGTKLEIKR

Recombinant full-length human IgG/kappa of humanized LYV392 antibodies were constructed. The humanized LYV392 antibodies include:
TM676 (including a heavy chain of VH-1/IgG1 mut and a light chain of VL-1/kappa)
TM677 (including a heavy chain of VH-1/IgG1 mut and a light chain of VL-2/kappa)

The amino acid sequences of the heavy chain and light chains of chimeric antibody TM392 (LYV392 with human constant regions) and the humanized anti-GITR antibodies listed above are provided below:

TM392
Heavy chain (SEQ ID NO: 73):
QVQLKESGPGLVQPSETLSLTCNVSGFSLTSYNVHWVRQPPGKGLEWLG
VIWSGVRTDYNSVLKSRLSIRWDSSKNQVFLKMNSLQSEDTATYYCARG
TYDANYHDVMDAWGQGASVTVSSASTKGPSVFPLAPCSRSTSESTAALG
CLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKS
LSLSLGK
Light chain (SEQ ID NO: 74):
DTVLTQSPALAVSPGERVTISCRASKSVRTGMHWYQQKPGQQPKLLVYG
ASNLESGVPARFSGSGSGTDFTLTIDPVEADDIATYFCHQSWNHFTFGS
GTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTH
QGLSSPVTKSFNRGEC
TM676
Heavy chain (SEQ ID NO: 75):
QVQLQESGPGLVKPSETLSLTCTVSGFSLTSYNVHWIRQPPGKGLEWIG
VIWSGVRTDYNSVLKSRVTISVDTSKNQFSLKLSSVTAADTAVYYCARG
TYDANYHDVMDAWGQGTMVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGPSVFL
FPPKPKDTLMISRTPEVTCVVVDVEHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
Light chain (SEQ ID NO: 76):
EIVLTQSPATLSLSPGERATLSCRASKSVRTGMHWYQQKPGQAPRLLIY
GASNLESGIPARFSGSGSGTDFTLTISSLEPEDFAVYYCHQSWNHFTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
TM677
Heavy chain (SEQ ID NO: 75):
same as TM676
Light chain (SEQ ID NO: 77):
DTVLTQSPATLSLSPGERATLSCRASKSVRTGMHWYQQKPGQAPRLLVY
GASNLESGIPARFSGSGSGTDFTLTISSLEPEDFATYFCHQSWNHFTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

Humanized Anti-GITR Antibodies Derived from LYV396

Sequence alignments were performed to compare the LYV396 V_{H} and V_{L} to human germline V_{H} and V_{L} sequences, respectively, following methods known in the art. See, e.g., Glanville J. et al. PNAS 2009; 106 (48) 20216-21. Based on overall sequence identity, matching interface positions and similarly classed CDR canonical positions, a germline family was identified for each of the light and heavy chains as the desired acceptor frameworks, i.e., IGKV3-11*01 for the light chain and IGHV4-59*01 for the heavy chain. Human acceptors were identified as ANV21835.1 immunoglobulin kappa light chain and AAV40120.1 immunoglobulin heavy chain variable region, the amino acid sequences of which are shown below:

>AAV40120.1 Human VH Acceptor
(SEQ ID NO: 78)
MKHLWFFLLLVAAPRWVLSQVQLQESGPGLVKPSETLSLTCTVSGGSIS
SYYWSWIRQPPGKGLEWIGYIYYSGSTNYNPSLKSRVTISVDTSKNQFS
LKLSSVTAADTAVYYCARGRHDYGDYFDYWGQGTLVTVSSGSASAPTLF
PLVSCENSPSDTSK
>ANV21835.1 Human VL Acceptor
(SEQ ID NO: 79)
MEAPAQLLFLLLLWLPDTTGEIVLTQSPATLSLSPGERATLSCRASQSV
SSYLAWYQQKPGQAPRLLIYDASNRATGIPARFSGSGSGTDFTLTISSL
EPEDFAVYYCQQRSNWRTFGQGTKLEIKRTVAAPSVFIFPPSDEQLKSG
TASVVCLLNNFYP

The CDRs of the parent LYV396 antibody were grafted into the corresponding CDR regions of the above-noted human V_{H} and V_{L} acceptor sequences to generate humanized LYV396_VH-1 and LYV396_VL-1 chains (grafted humanized antibody), the amino acid sequence of each of which is provided below (CDRs in boldface):

>LYV396_VH-1 (grafted LYV396_VH, SEQ ID NO: 80)
QVQLQESGPGLVKPSETLSLTCTVSGFSLTSYNVHWIRQPPGKGLEWIG
VIWSGVRTDYNSVLKPRVTISVDTSKNQFSLKLSSVTAADTAVYYCARG
TYDDNYHDVMDAWGQGTLVTVSS
>LYV396_VL-1 (grafted LYV396_VL; SEQ ID NO: 81)
EIVLTQSPATLSLSPGERATLSCRASKSVRTGMHWYQQKPGQAPRLLIY
GASNLESGIPARFSGSGSGTDFTLTISSLEPEDFAVYYCQQSWNHFTFG
QGTKLEIKR

Recombinant full human IgG/kappa of humanized LYV396 antibodies were constructed. The humanized LYV396 antibodies include:
TM685 (including a heavy chain of LYV396_VH-1/IgG1mut and a light chain of LYV396_VL-1/kappa),

The amino acid sequences of the heavy chain and light chain of the chimeric antibody TM396 (IgG4) and the anti-GITR humanized antibodies derived from LYV396 listed above are provided below:

TM396
Heavy chain (SEQ ID NO: 82):
QVQLKESGPGLVQPSETLSLTCTVSGFSLTSYNVHWVRQPPGKGLEWLG
VIWSGVRTDYNSVLKPRLSISRDSSKNKVFLNMNSLQSEDTATYYCARG
TYDDNYHDVMDAWGQGASVTVSSASTKGPSVFPLAPCSRSTSESTAALG
CLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKS
LSLSLGK
Light Chain (SEQ ID NO: 83):
DTVLTQSPALAVSPGERVTISCRASKSVRTGMHWYQQKPGQQPKLLIYG
ASNLESGVPVRFSGSGSGTDFTLTIDPVEADDTATYFCQQSWNHFTFGS
GTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTH
QGLSSPVTKSFNRGEC
TM685
Heavy Chain ((SEQ ID NO: 84):
QVQLQESGPGLVKPSETLSLTCTVSGFSLTSYNVHWIRQPPGKGLEWIG
VIWSGVRTDYNSVLKPRVTISVDTSKNQFSLKLSSVTAADTAVYYCARG
TYDDNYHDVMDAWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGPSVFL
FPPKPKDTLMISRTPEVTCVVVDVEHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
Light Chain (SEQ ID NO: 85):
EIVLTQSPATLSLSPGERATLSCRASKSVRTGMHWYQQKPGQAPRLLIY
GASNLESGIPARFSGSGSGTDFTLTISSLEPEDFAVYYCQQSWNHFTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

### (ii) Characterization of Anti-GITR Antibodies

(a) Binding Activity to Cell Surface GITR

FACS analysis was performed to evaluate the binding properties of exemplary anti-GITR humanized antibodies. Briefly, CHO cells over-expressing human GITR were harvested using trypsin-EDTA partial digestion followed by centrifugation at 1000 g for 3 minutes. The cells were re-suspended in cold PBS-BSA (2%) at 2×10⁶/mL and aliquoted to 100 μL/tube. The anti-GITR humanized antibodies were diluted in PBS-BSA (final concentrations were 0.01, 0.1, 1 and 10 μg/mL) and 50 μL of each was added to the CHO-GITR cells. The cell solutions were mixed and incubated at 4° C. in the dark for 2 hours. The cells were then washed with PBS-BSA twice. Secondary antibody conjugates (goat F(ab′)2 anti-human IgG—Fc (PE), pre-adsorbed, Abcam #ab98596) at 1/500 dilution and 100 μL/well was added and the cells were mixed and incubated 4° C. in dark for 1 hour. The cells were then washed twice with PBS-BSA, followed by fixation in 2% PFA/PBS, and were then subjected to FACS analysis.

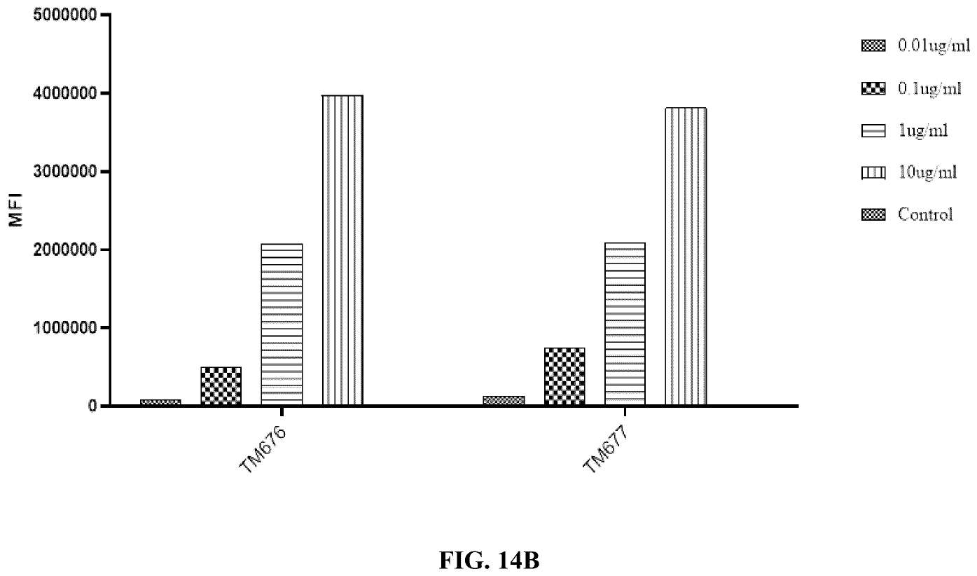

Binding of the antibodies to human GITR expressing CHO cells were evaluated and the mean fluorescence intensity is plotted in histograms or dot plots as shown in FIGS. 14A-14B. Both humanized versions of the anti-GITR antibody LYV392 showed similar binding activity to the cell surface GITR as chimeric TM392. Humanized LYV396 antibody TM685 showed more potent binding than chimeric TM396.
(b) Agonistic Activity for GITR

To determine the agonist activity of the anti-GITR antibodies, a GITR reporter assay was developed, which involves reporter cells over-expressing human GITR. This GS-H2-huGITR reporter cells were re-suspended and diluted to 5×10⁴ cells/mL with assay buffer (MEM containing 1% FBS). The cells were added at 100 μL/well, such that the final cell number was 5000 cells/well in the assay plate (Nunc, Cat #167425). Samples were added at 100 uL/well test sample at 2× final concentrations to the assay plate. The assay plate was incubated in 37° C., 5% CO₂ incubator for 18-20 hours. After the 18-20 hour incubation, 8 μL of the supernatant from each well of the assay plate was collected and added to HTRF detection assay plate (Nunc). A Human Interleukin 8 (reporter of GITR activation) detection assay was performed using a Human IL-8 Assay Kit (Cisbio, Cat #62IL8PEB). In particular, 16 μL assay volume was used. The results were read using Time Resolved Fluorescence by Tecan F200pro and the relative light unit data was recorded.

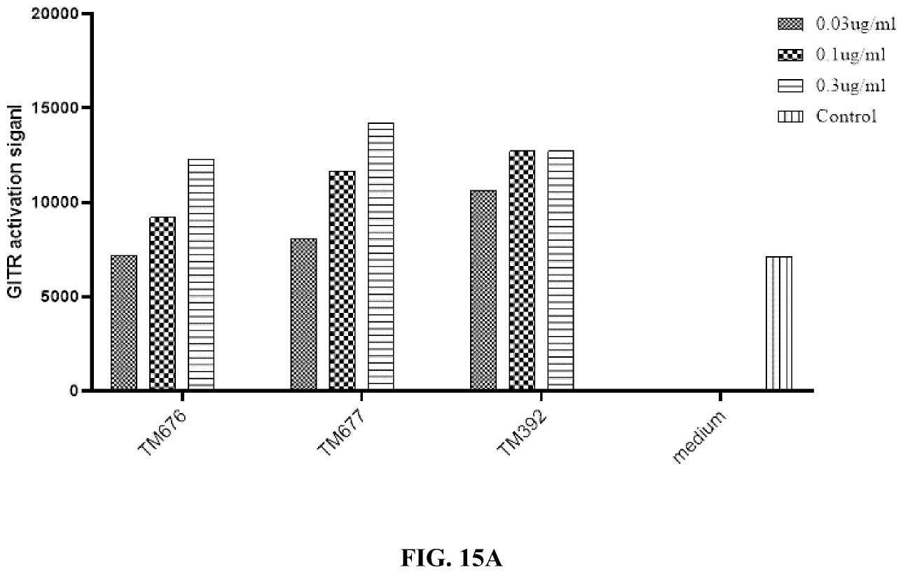

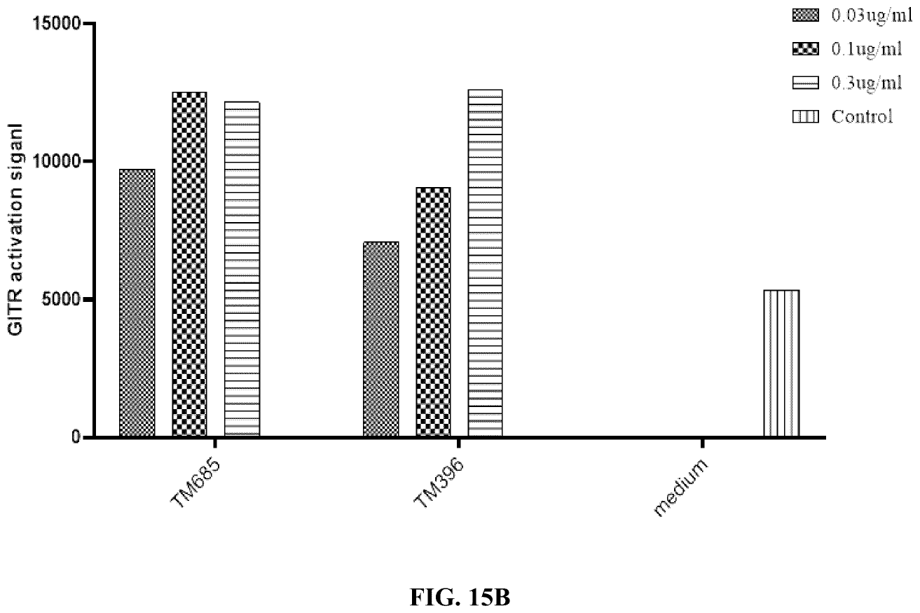

As shown in FIGS. 15A-15B, the anti-GITR antibodies stimulated human GITR activation as evidenced by secretion levels of IL-8 in the reporter assays. The chimeric antibodies TM392 and TM396, and the humanized antibodies TM676, TM677 and TM685 showed highly potent GITR agonist activity.

(c) Anti-Tumor Activity

Exemplary humanized anti-GITR antibodies were tested in mouse syngeneic tumor models in vivo to determine the anti-tumor efficacy and toxicity of these antibodies. Murine colon cancer MC38 tumor cells were subcutaneously implanted into homozygous human GITR knock-in C57BL/6 mice. Mice were grouped when the tumor size was approximately 150±50 mm³ (n=6). Humanized anti-GITR antibodies were administered by intraperitoneal injections and tumor sizes were measure during 4-6 weeks of antibody treatment. Tumor sizes were calculated as tumor volume using formula of 0.5×length×width².

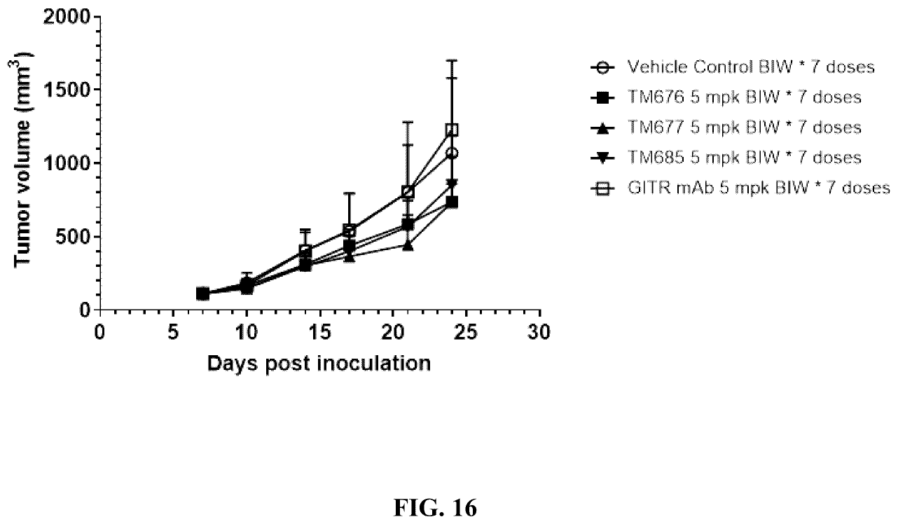

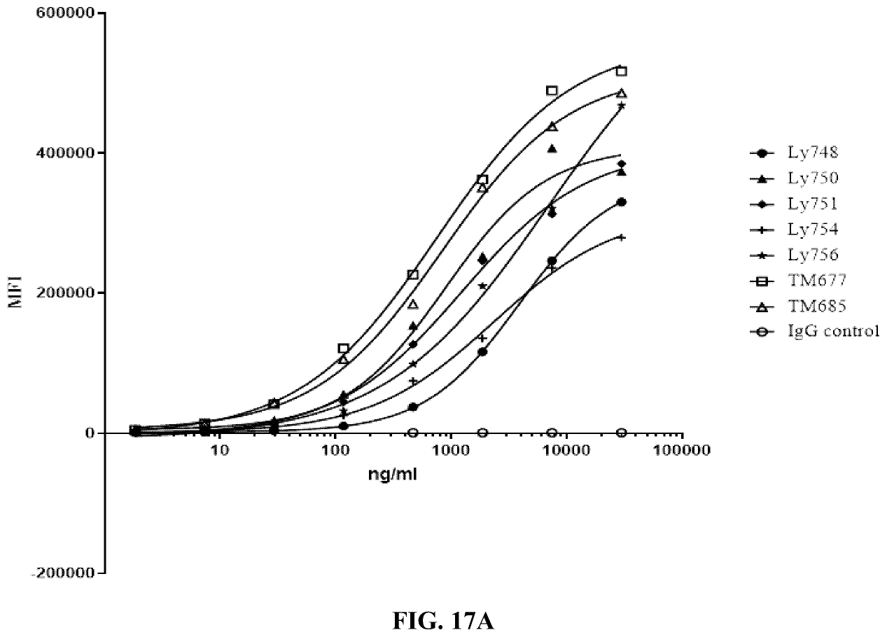

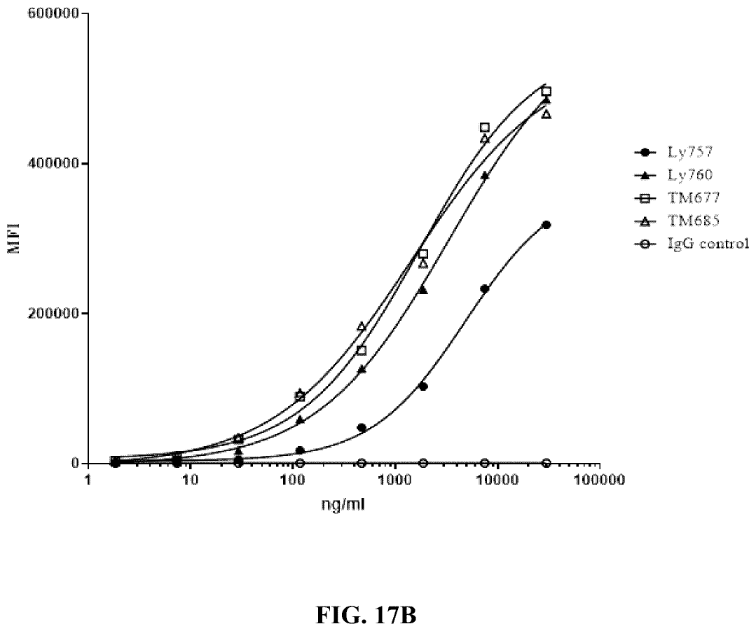

Anti-tumor efficacy was evaluated between tumor sizes of the control group and antibody treatment group as shown in FIG. 16. Exemplary clones TM676, TM677 and TM685 showed anti-tumor activities as compared with the control and a GITR reference mAb (described in WO 2011/028683) groups.

(iii) Preparation of Anti-GITR/CD137 Bi-Specific Antibodies

Anti-GITR/CD137 bi-specific antibodies were produced using the anti-CD137 antibody Ly1630 and anti-GJTR antibodies TM677 and TM685, all of which are humanized antibodies. cDNAs encoding the V_{H} and V_{L} chains of those anti-CD137 and anti-GITR antibodies (sequences provided above) were used as the starting materials for constructing anti-GITR/CD137 bispecific antibodies. CHO-cell transient expression was carried out with plasmids configured for expressing polypeptide chains of the bi-specific antibodies. These antibodies were purified by protein A affinity chromatography.

The amino acid sequences of the polypeptides of the bi-specific antibodies are provided below:

Ly746
First polypeptide (SEQ ID NO: 86):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSDTVLTQSPATLSLSPGERA
TLSCRASKSVRTGMHWYQQKPGQAPRLLVYGASNLESGIPARFSGSGSGTDFTLTISSLEPEDFATYFC
HQSWNHFTFGQGTKLEIKGGGGSGGGGSGGGGSGGGGSQVQLQESGPGLVKPSETLSLTCTVSGFSLTS
YNVHWIRQPPGKGLEWIGVIWSGVRTDYNSVLKSRVTISVDTSKNQFSLKLSSVTAADTAVYYCARGTY
DANYHDVMDAWGQGTMVTVSS
Second polypeptide (SEQ ID NO: 14):
same as the second polypeptide of Ly461
Ly747
First polypeptide (SEQ ID NO: 87):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSQVQLQESGPGLVKPSETLS
LTCTVSGFSLTSYNVHWIRQPPGKGLEWIGVIWSGVRTDYNSVLKSRVTISVDTSKNQFSLKLSSVTAA
DTAVYYCARGTYDANYHDVMDAWGQGTMVTVSSGGGGSGGGGSGGGGSGGGGSDTVLTQSPATLSLSPG
ERATLSCRASKSVRTGMHWYQQKPGQAPRLLVYGASNLESGIPARFSGSGSGTDFTLTISSLEPEDFAT
YFCHQSWNHFTFGQGTKLEIK
Second polypeptide: (SEQ ID NO: 14):
same as the second polypeptide of Ly461
Ly748
First polypeptide: (SEQ ID NO: 88):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Second polypeptide (SEQ ID NO: 89):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSDTVLTQSPATLSLSPGERATLSCRASKSVRTGMHWYQQKPGQ
APRLLVYGASNLESGIPARFSGSGSGTDFTLTISSLEPEDFATYFCHQSWNHFTFGQGTKLEIKGGGGS
GGGGSGGGGSGGGGSQVQLQESGPGLVKPSETLSLTCTVSGFSLTSYNVHWIRQPPGKGLEWIGVIWSG
VRTDYNSVLKSRVTISVDTSKNQFSLKLSSVTAADTAVYYCARGTYDANYHDVMDAWGQGTMVTVSS
Ly749
First polypeptide: (SEQ ID NO: 88):
same as the first polypeptide of Ly748
Second polypeptide (SEQ ID NO: 90):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSQVQLQESGPGLVKPSETLSLTCTVSGFSLTSYNVHWIRQPPG
KGLEWIGVIWSGVRTDYNSVLKSRVTISVDTSKNQFSLKLSSVTAADTAVYYCARGTYDANYHDVMDAW
GQGTMVTVSSGGGGSGGGGSGGGGSGGGGSDTVLTQSPATLSLSPGERATLSCRASKSVRTGMHWYQQK
PGQAPRLLVYGASNLESGIPARFSGSGSGTDFTLTISSLEPEDFATYFCHQSWNHFTFGQGTKLEIK
Ly750
First polypeptide (SEQ ID NO: 91):
QVQLQESGPGLVKPSETLSLTCTVSGFSLTSYNVHWIRQPPGKGLEWIGVIWSGVRTDYNSVLKSRVTI
SVDTSKNQFSLKLSSVTAADTAVYYCARGTYDANYHDVMDAWGQGTMVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSGGGGSGGGGSDIQMTQSPSSLSAS
VGDRVTITCRASQDIRSNLNWYQQKPGGAVKLLIYYTSRLHSGVPSRFSGSGSGTDYTLTISSLQPEDE
ATYFCQQSEKLPRTFGGGTKVEIRGGGGSGGGGSGGGGSGGGGSQVQLVQSGAEVKKPGASVKVSCKAS
GYTFAGFEMHWVRQAPGQGLEWMGAIDPKTGGTDYNQKFKDRVTMTRDTSISTAYMELSRLRSDDTAVY
YCARDLGYFDVWGQGTLVTVSS
Second polypeptide: (SEQ ID NO: 78):
same as the light chain of TM677
Ly751
First polypeptide (SEQ ID NO: 92):
QVQLQESGPGLVKPSETLSLTCTVSGFSLTSYNVHWIRQPPGKGLEWIGVIWSGVRTDYNSVLKSRVTI
SVDTSKNQFSLKLSSVTAADTAVYYCARGTYDANYHDVMDAWGQGTMVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSGGGGSGGGGSQVQLVQSGAEVKKP
GASVKVSCKASGYTFAGFEMHWVRQAPGQGLEWMGAIDPKTGGTDYNQKFKDRVTMTRDTSISTAYMEL
SRLRSDDTAVYYCARDLGYFDVWGQGTLVTVSSGGGGSGGGGSGGGGSGGGGSDIQMTQSPSSLSASVG
DRVTITCRASQDIRSNLNWYQQKPGGAVKLLIYYTSRLHSGVPSRFSGSGSGTDYTLTISSLQPEDFAT
YFCQQSEKLPRTFGGGTKVEIR
Second polypeptide (SEQ ID NO: 77):
same as the light chain of TM677
Ly752
First polypeptide (SEQ ID NO: 93):
QVQLQESGPGLVKPSETLSLTCTVSGFSLTSYNVHWIRQPPGKGLEWIGVIWSGVRTDYNSVLKSRVTI
SVDTSKNQFSLKLSSVTAADTAVYYCARGTYDANYHDVMDAWGQGTMVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
Second polypeptide (SEQ ID NO: 94):
DTVLTQSPATLSLSPGERATLSCRASKSVRTGMHWYQQKPGQAPRLLVYGASNLESGIPARFSGSGSGT
DFTLTISSLEPEDFATYFCHQSWNHFTFGQGTKLEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSDIQMTQSPSSLSASVGDRVTITCRASQDIRSNLNWYQQKPGGA
VKLLIYYTSRLHSGVPSRFSGSGSGTDYTLTISSLQPEDFATYFCQQSEKLPRTFGGGTKVEIRGGGGS
GGGGSGGGGSGGGGSQVQLVQSGAEVKKPGASVKVSCKASGYTFAGFEMHWVRQAPGQGLEWMGAIDPK
TGGTDYNQKFKDRVTMTRDTSISTAYMELSRLRSDDTAVYYCARDLGYFDVWGQGTLVTVSS
Ly753
First polypeptide (SEQ ID NO: 93):
same as the first polypeptide of Ly752
Second polypeptide (SEQ ID NO: 95):
DTVLTQSPATLSLSPGERATLSCRASKSVRTGMHWYQQKPGQAPRLLVYGASNLESGIPARFSGSGSGT
DFTLTISSLEPEDFATYFCHQSWNHFTFGQGTKLEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSQVQLVQSGAEVKKPGASVKVSCKASGYTFAGFEMHWVRQAPGQ
GLEWMGAIDPKTGGTDYNQKFKDRVTMTRDTSISTAYMELSRLRSDDTAVYYCARDLGYFDVWGQGTLV
TVSSGGGGSGGGGSGGGGSGGGGSDIQMTQSPSSLSASVGDRVTITCRASQDIRSNLNWYQQKPGGAVK
LLIYYTSRLHSGVPSRFSGSGSGTDYTLTISSLQPEDFATYFCQQSEKLPRTFGGGTKVEIR
Ly754
First polypeptide (SEQ ID NO: 96):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSEIVLTQSPATLSLSPGERA
TLSCRASKSVRTGMHWYQQKPGQAPRLLIYGASNLESGIPARFSGSGSGTDFTLTISSLEPEDFAVYYC
QQSWNHFTFGQGTKLEIKGGGGSGGGGSGGGGSGGGGSQVQLQESGPGLVKPSETLSLTCTVSGFSLTS
YNVHWIRQPPGKGLEWIGVIWSGVRTDYNSVLKPRVTISVDTSKNQFSLKLSSVTAADTAVYYCARGTY
DDNYHDVMDAWGQGTLVTVSS
Second polypeptide (SEQ ID NO: 14):
same as the second polypeptide of Ly461
Ly755
First polypeptide (SEQ ID NO: 97):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSQVQLQESGPGLVKPSETLS
LTCTVSGFSLTSYNVHWIRQPPGKGLEWIGVIWSGVRTDYNSVLKPRVTISVDTSKNQFSLKLSSVTAA
DTAVYYCARGTYDDNYHDVMDAWGQGTLVTVSSGGGGSGGGGSGGGGSGGGGSEIVLTQSPATLSLSPG
ERATLSCRASKSVRTGMHWYQQKPGQAPRLLIYGASNLESGIPARFSGSGSGTDFTLTISSLEPEDFAV
YYCQQSWNHFTFGQGTKLEIK
Second polypeptide: (SEQ ID NO: 14):
same as the second polypeptide of Ly461.
Ly756
First polypeptide (SEQ ID NO: 88):
same as the first polypeptide of Ly748.
Second polypeptide (SEQ ID NO: 98):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSEIVLTQSPATLSLSPGERATLSCRASKSVRTGMHWYQQKPGQ
APRLLIYGASNLESGIPARFSGSGSGTDFTLTISSLEPEDFAVYYCQQSWNHFTFGQGTKLEIKGGGGS
GGGGSGGGGSGGGGSQVQLQESGPGLVKPSETLSLTCTVSGFSLTSYNVHWIRQPPGKGLEWIGVIWSG
VRTDYNSVLKPRVTISVDTSKNQFSLKLSSVTAADTAVYYCARGTYDDNYHDVMDAWGQGTLVTVSS
Ly757
First polypeptide (SEQ ID NO: 88):
same as the first polypeptide of Ly748.
Second polypeptide (SEQ ID NO: 99):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSQVQLQESGPGLVKPSETLSLTCTVSGFSLTSYNVHWIRQPPG
KGLEWIGVIWSGVRTDYNSVLKPRVTISVDTSKNQFSLKLSSVTAADTAVYYCARGTYDDNYHDVMDAW
GQGTLVTVSSGGGGSGGGGSGGGGSGGGGSEIVLTQSPATLSLSPGERATLSCRASKSVRTGMHWYQQK
PGQAPRLLIYGASNLESGIPARFSGSGSGTDFTLTISSLEPEDFAVYYCQQSWNHFTFGQGTKLEIK
Ly758
First polypeptide (SEQ ID NO: 100):
QVQLQESGPGLVKPSETLSLTCTVSGFSLTSYNVHWIRQPPGKGLEWIGVIWSGVRTDYNSVLKPRVTI
SVDTSKNQFSLKLSSVTAADTAVYYCARGTYDDNYHDVMDAWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSGGGGSGGGGSDIQMTQSPSSLSAS
VGDRVTITCRASQDIRSNLNWYQQKPGGAVKLLIYYTSRLHSGVPSRFSGSGSGTDYTLTISSLQPEDF
ATYFCQQSEKLPRTFGGGTKVEIRGGGGSGGGGSGGGGSGGGGSQVQLVQSGAEVKKPGASVKVSCKAS
GYTFAGFEMHWVRQAPGQGLEWMGAIDPKTGGTDYNQKFKDRVTMTRDTSISTAYMELSRLRSDDTAVY
YCARDLGYFDVWGQGTLVTVSS
Second polypeptide (SEQ ID NO: 85):
same as the light chain of TM685
Ly759
First polypeptide (SEQ ID NO: 101):
QVQLQESGPGLVKPSETLSLTCTVSGFSLTSYNVHWIRQPPGKGLEWIGVIWSGVRTDYNSVLKPRVTI
SVDTSKNQFSLKLSSVTAADTAVYYCARGTYDDNYHDVMDAWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSGGGGSGGGGSQVQLVQSGAEVKKP
GASVKVSCKASGYTFAGFEMHWVRQAPGQGLEWMGAIDPKTGGTDYNQKFKDRVTMTRDTSISTAYMEL
SRLRSDDTAVYYCARDLGYFDVWGQGTLVTVSSGGGGSGGGGSGGGGSGGGGSDIQMTQSPSSLSASVG
DRVTITCRASQDIRSNLNWYQQKPGGAVKLLIYYTSRLHSGVPSRFSGSGSGTDYTLTISSLQPEDFAT
YFCQQSEKLPRTFGGGTKVEIR
Second polypeptide (SEQ ID NO: 85):
same as the light chain of TM685
Ly760
First polypeptide (SEQ ID NO: 102):
QVQLQESGPGLVKPSETLSLTCTVSGFSLTSYNVHWIRQPPGKGLEWIGVIWSGVRTDYNSVLKPRVTI
SVDTSKNQFSLKLSSVTAADTAVYYCARGTYDDNYHDVMDAWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
Second polypeptide (SEQ ID NO: 103):
EIVLTQSPATLSLSPGERATLSCRASKSVRTGMHWYQQKPGQAPRLLIYGASNLESGIPARFSGSGSGT
DFTLTISSLEPEDFAVYYCQQSWNHFTFGQGTKLEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSDIQMTQSPSSLSASVGDRVTITCRASQDIRSNLNWYQQKPGGA
VKLLIYYTSRLHSGVPSRFSGSGSGTDYTLTISSLQPEDFATYFCQQSEKLPRTFGGGTKVEIRGGGGS
GGGGSGGGGSGGGGSQVQLVQSGAEVKKPGASVKVSCKASGYTFAGFEMHWVRQAPGQGLEWMGAIDPK
TGGTDYNQKFKDRVTMTRDTSISTAYMELSRLRSDDTAVYYCARDLGYFDVWGQGTLVTVSS
Ly761
First polypeptide (SEQ ID NO: 102):
same as the first polypeptide of Ly760
Second polypeptide (SEQ ID NO: 104):
EIVLTQSPATLSLSPGERATLSCRASKSVRTGMHWYQQKPGQAPRLLIYGASNLESGIPARFSGSGSGT
DFTLTISSLEPEDFAVYYCQQSWNHFTFGQGTKLEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKS
FNRGECGGGGSGGGGSGGGGSGGGGSQVQLVQSGAEVKKPGASVKVSCKASGYTFAGFEMHWVRQAPGQ
GLEWMGAIDPKTGGTDYNQKFKDRVTMTRDTSISTAYMELSRLRSDDTAVYYCARDLGYFDVWGQGTLV
TVSSGGGGSGGGGSGGGGSGGGGSDIQMTQSPSSLSASVGDRVTITCRASQDIRSNLNWYQQKPGGAVK
LLIYYTSRLHSGVPSRFSGSGSGTDYTLTISSLQPEDFATYFCQQSEKLPRTFGGGTKVEIR

### Characterization of Anti-GITR/CD137 Bi-Specific Antibodies

(i) Binding Activity

Anti-GITR/CD137 bi-specific antibodies were analyzed by FACS for their binding properties to human GITR and/or human CD137 expressed on CHO cells. Briefly, cultured cells were harvested, counted and cell viability was evaluated using the Trypan Blue exclusion method. Viable cells were then adjusted to 2×10⁶ cells per mL in PBS containing 2% BSA. 100 μL of this cell suspension were further aliquoted per well into a V-bottom 96-well plate. 50 μL of the bi-specific antibodies or corresponding IgG control were added to the cell-containing wells to obtain final concentrations of 0.1 μg/mL to 10 μg/mL. After incubation for 2 hours at 4° C., cells were centrifuged (3 min, 1000× g), washed with 250 μL/well BSA-containing FACS Stain Buffer, resuspended and incubated for an additional 1 hour at 4° C. with 100 μL/well fluorochrome-conjugated anti-IgG antibody for detection of the bisepecific antibody. Cells were then washed with 250 μL/well BSA-containing FACS Stain Buffer, resuspended in 100 μL/well FACS Stain Buffer, acquired and analyzed using a FACS machine. Binding of the bispecific antibodies to human GITR or human CD137 expressing CHO cells were evaluated and the mean fluorescence intensity is plotted in histograms or dot plots.

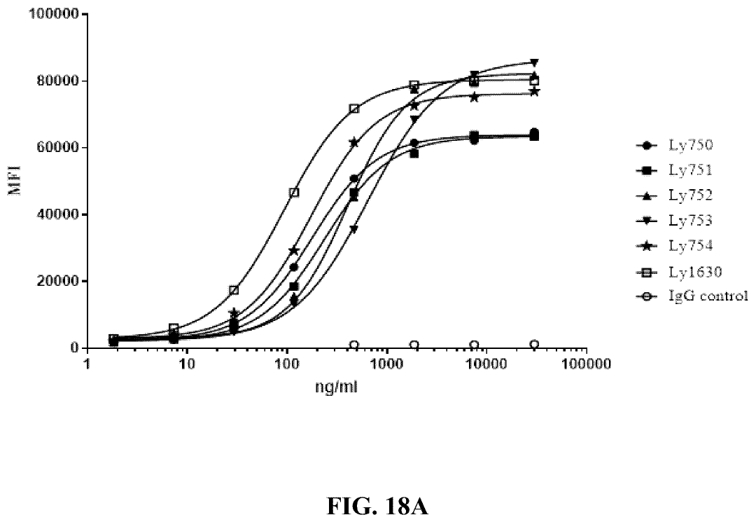

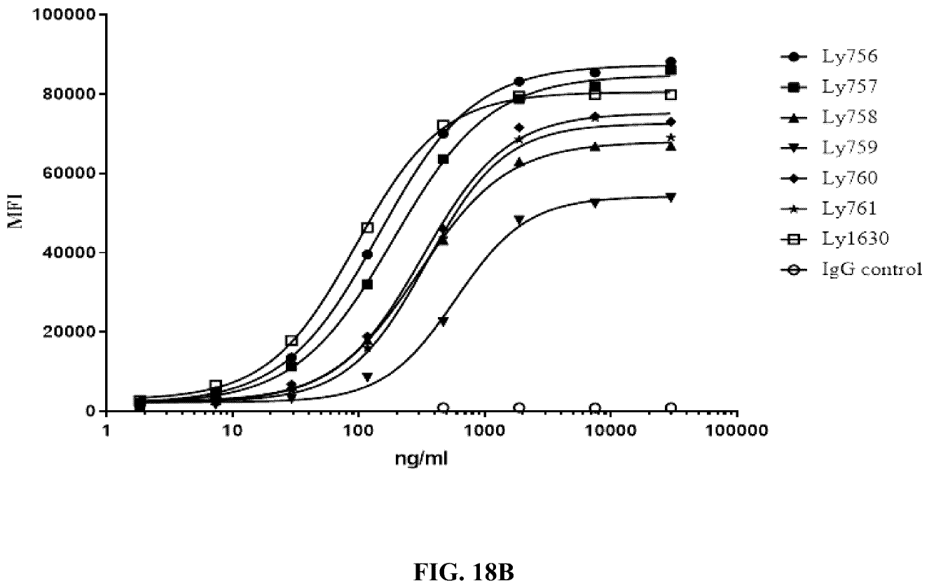

As shown in FIGS. 17A and 17B, the exemplary anti-GITR/CD137 bi-specific antibodies exhibited a various range of binding affinity to human GITR expressed on the CHO cells. As shown in FIGS. 18A and 18B, the bi-specific antibodies exhibited different levels of binding affinity to human CD137 expressed on CHO cells.

(ii) Agonistic Activity for CD137

To determine the agonist activity of these anti-GITR/CD137 bi-specific antibodies, a CD137 reporter assay was developed, which involves reporter cells over-expressing human CD137. The CD137 reporter assay was performed in co-culture with GITR-expressing CHO cells following the procedures as described in Example 1.

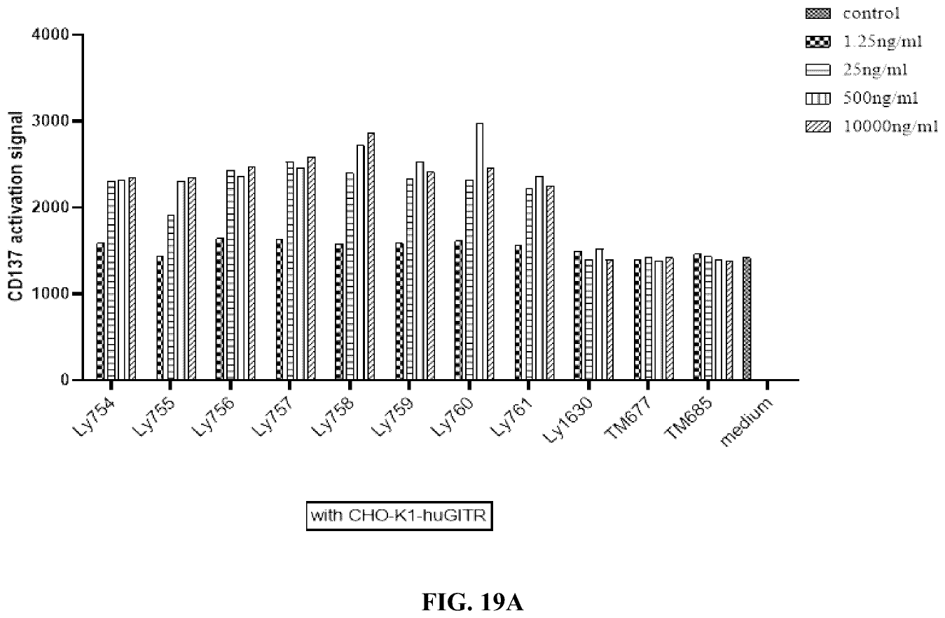

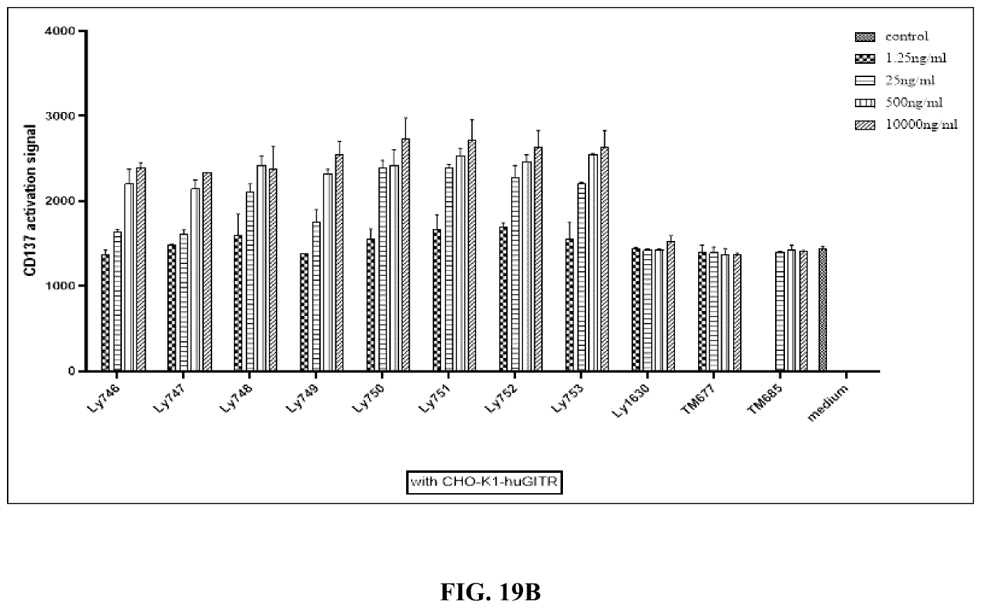

As shown in FIGS. 19A and 19B, the tested exemplary bi-specific antibodies showed CD137 agonist activity in presence of human GITR expressing cells, as compared to the nondetectable activity of their parental anti-CD137 mAb. Binding to both CD137 and GITR by the tested bi-specific antibodies simultaneously in a microenvironment would affect individual binding due to the avidity effect, which refers to the accumulated strength of multiple affinities of individual non-covalent binding interactions.

(iii) Agonistic Activity for GITR

To determine the agonist activity of these anti-GITR/CD137 bi-specific antibodies, a GITR reporter assay was developed, which involves reporter cells over-expressing human GITR. The GITR reporter assay was performed in co-culture with CD137-expressing CHO cells following the procedures as described in Example 1.

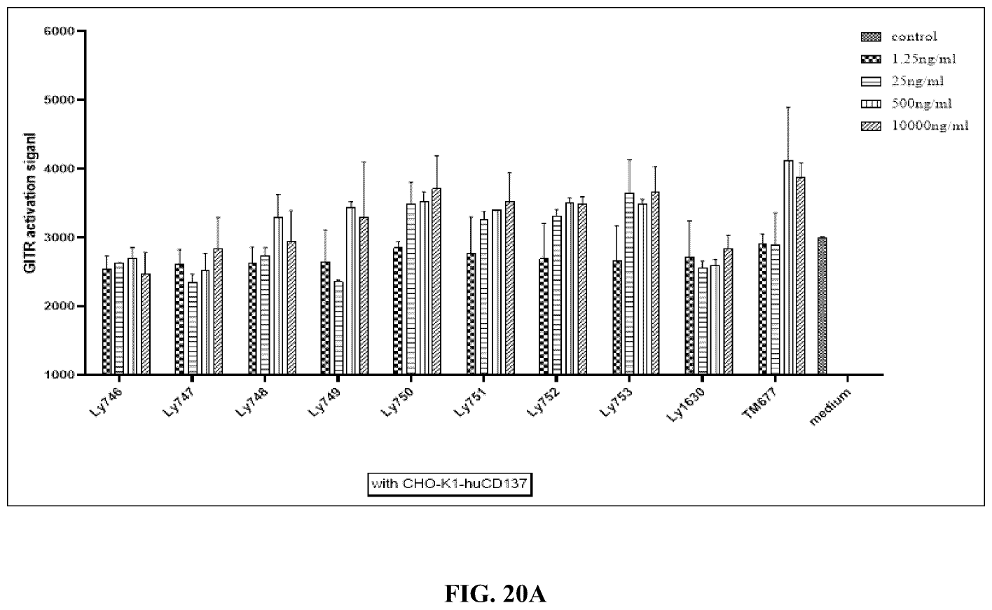

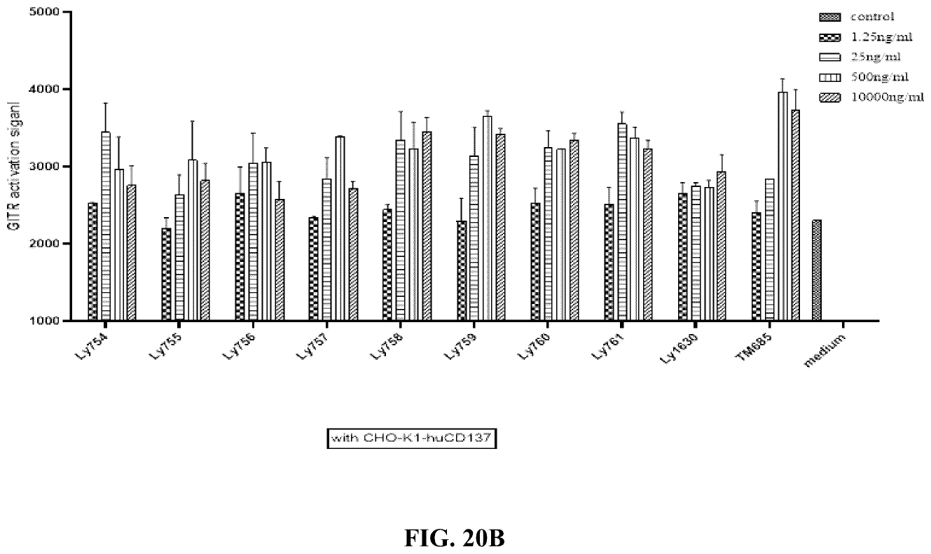

As shown in FIGS. 20A and 20B, the tested examplary bi-specific antibodies showed a range of GITR agonist activity in presence of human CD137 expressing cells, as compared with that of their parental anti-GITR mAbs.

(iv) PBMC Activation

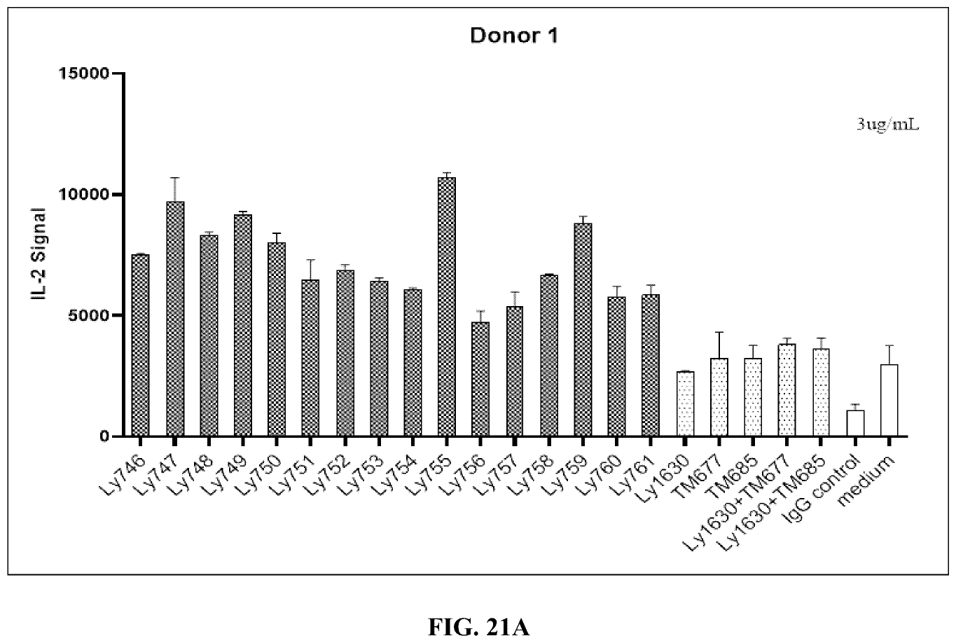

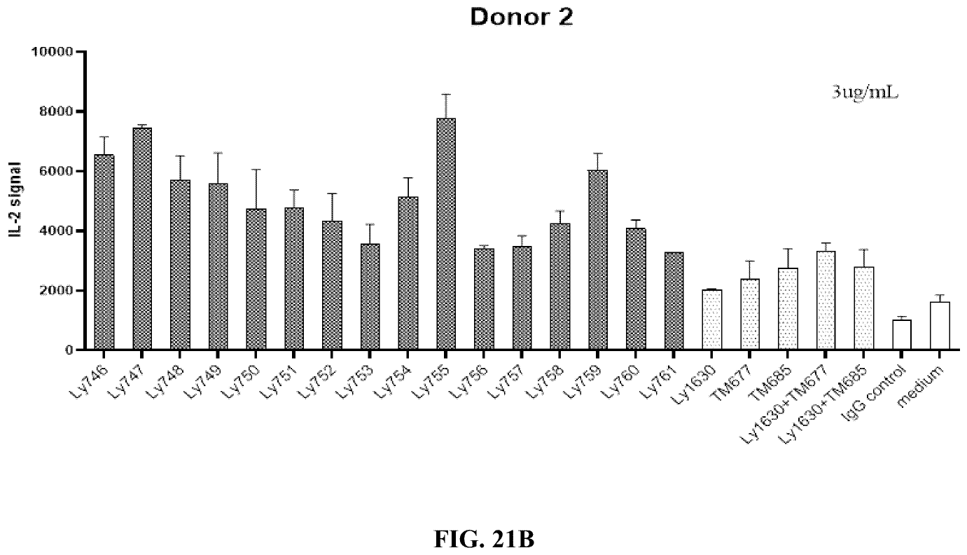

A PBMC activation assay was performed to show the co-stimulation functionality of the bispecific antibodies. Briefly, 2×10⁵ PBMCs in culture medium added SEB (final concentration at 0.01 μg/mL) and serial diluted antibody samples were added to plates and incubated at 37° C. with 5% CO₂ for 5 days. Cell culture supernatants were then collected for cytokine detection using Human IL-2 detection kit following the instruction manual. FIGS. 21A and 21B shows that the exemplary bispecific antibodies induced stronger IL-2 production from human PBMCs than anti-GITR or anti-CD137 mAbs alone or even in combination. Therefore, binding to CD137 and GITR by antibody molecules simultaneously in a microenvironment would enhance T cell co-stimulation activity of these bi-specific antibodies compared with their parental mAbs.

(v) Pharmacokinetic Studies of Anti-GITR/CD137 Bi-Specific Antibodies

C57BL/6 mice (6-7 weeks old, 19-20 g, female, purchased from Vital River) were used for the study. Antibodies were formulated in DPBS and administered via tail vein injection at 5 mg/kg in a group of 4 mice.

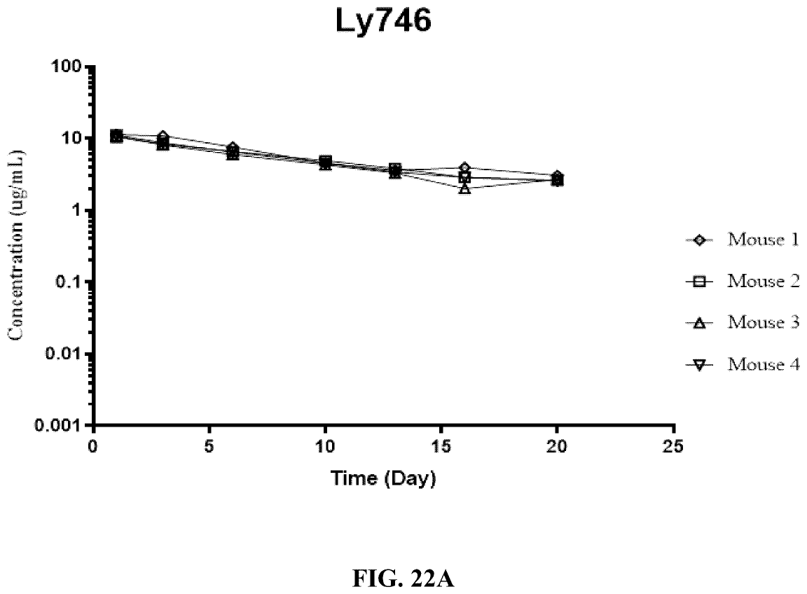

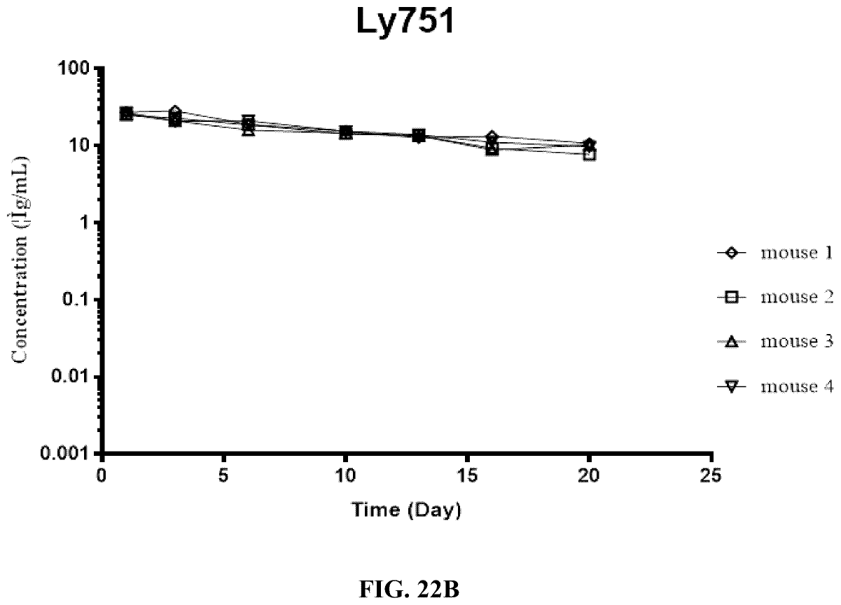

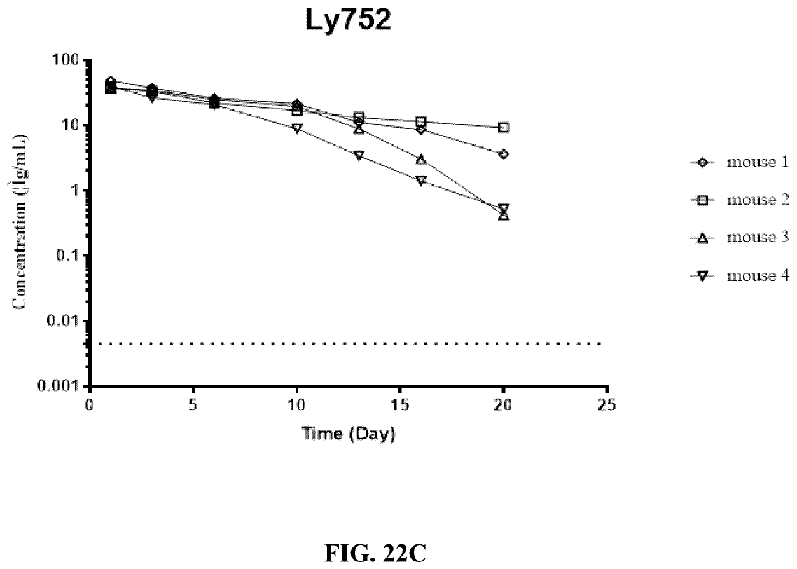

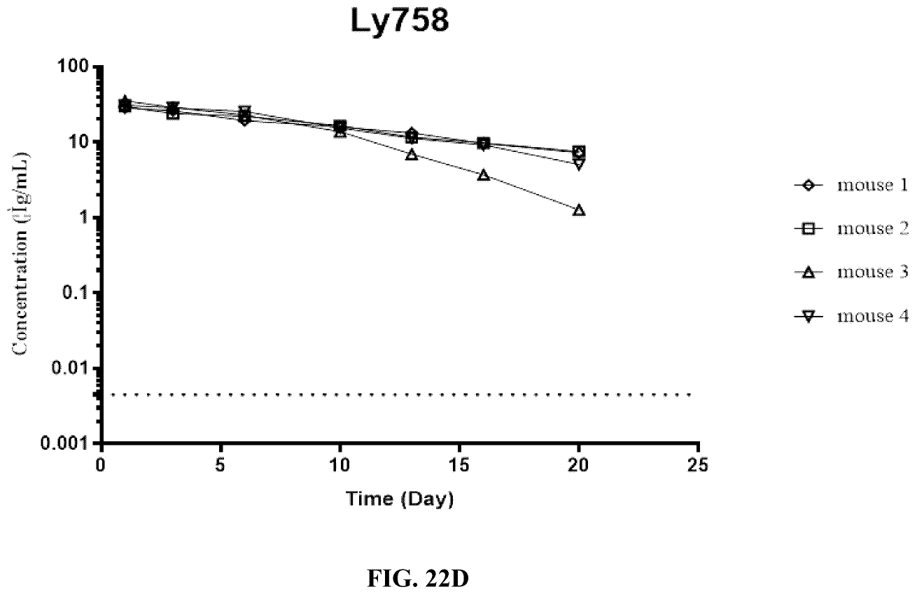

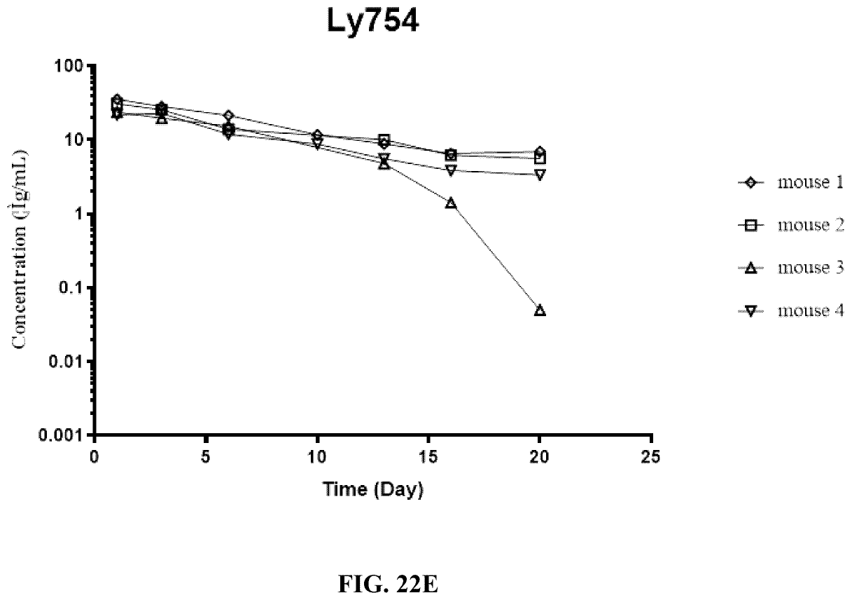

Blood sampling was done at pre-dose, Id, 4d, 7d, 10d, 14d, 17d and 21d by serial bleeding. 10 uL blood per time point was added to 40 uL of a PBS-BSA solution. The sample was then mixed well and centrifuged at 2000 g for 5 minutes at 4° C. The supernatant was put on dry ice immediately after collection and stored at approximately −70° C. until analysis. Blood antibody concentrations were determined by ELISA for simultaneous binding to GITR and CD137. FIGS. 22A-22E showed the blood concentrations of the bispecific antibodies after a single intravenous injection of 5 mg/kg. These bispecific antibodies showed high and lasting circulation concentrations.

(vi) Anti-Tumor Activity

Exemplary anti-GITR/CD137 bi-specific antibodies were tested in mouse syngeneic tumor models to determine their anti-tumor efficacy and toxicity. Briefly, murine melanoma B16-OVA tumor cells were subcutaneously implanted into mice received irradiation and mixed bone marrow transplantation from human CD137 knock-in C57BL/6 mice and human GITR knock-in C57BL/6 mice. Mice were grouped when the tumor size was approximately 150±50 mm³ (n=6). Exemplary anti-GITR/CD137 bi-specific antibodies were administered by intraperitoneal injections and tumor sizes were measure during 4-6 weeks of antibody treatment. Tumor sizes were calculated as tumor volume using formula of 0.5×length×width².

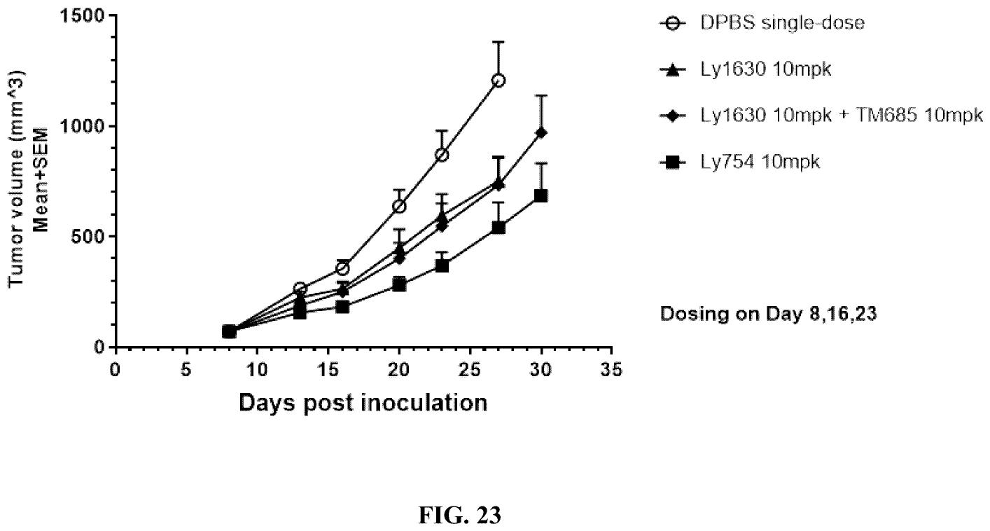

Anti-tumor efficacy was evaluated between tumor sizes of the control group and antibody treatment group as shown in FIG. 23. All exemplary bi-specific antibody Ly754 showed better anti-tumor activities as compared with the anti-PD1 and anti-CD137 parent clones, either taken alone or in combination.

### Example 4: Anti-CD40/CD137 Bi-Specific Antibodies

Anti-CD40/CD137 bi-specific antibodies were produced using the anti-CD137 antibody Ly1630 and anti-CD40 antibodies Ly253-G2. Structural information of Ly1630 is provided in Example 1 above. The amino acid sequences of Ly253-G2 are provided below.

Ly253-G2
Heavy chain (SEQ ID NO: 105):
QVQLVQSGAEVKKPGASVKVSCKASGYTFTGYYMHWVRQAPGQGLEWMG
WINPDSGGTNYAQKFQGRVTMTRDTSISTAYMELNRLRSDDTAVYYCAR
DQPLGYCTNGVCSYFDYWGQGTLVTVSSASTKGPSVFPLAPCSRSTSES
TAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISK
TKGQPREPQVYTLPPSREEMTKNQVSLTCLVKGFYPSDIAVEWESNGQP
ENNYKTTPPMLDSDGSFFLYSKLTVDKSRWQQGNVFSCSVMHEALHNHY
TQKSLSLSPG
V_{L} (SEQ ID NO: 106)
DIQMTQSPSSVSASVGDRVTITCRASQGIYSWLAWYQQKPGKAPNLLIY
TASTLQSGVPSRFSGSGSGTDFTLTISSLQPEDFATYYCQQANIFPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

cDNAs encoding the V_{H} and V_{L} chains of these anti-CD40 antibody and anti-CD137 antibody were used as the starting materials for making the bi-specific antibodies. CHO-cell transient expression was carried out with plasmids configured for expressing polypeptide chains of the bi-specific antibodies. These antibodies were purified by protein A affinity chromatography.

The amino acid sequences of the heavy chain (HC) and the light chain (LC) of the resultant bi-specific antibodies are provided below:

Ly738
First polypeptide (SEQ ID NO: 107):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSDIQMTQSPSSVSASVGDRV
TITCRASQGIYSWLAWYQQKPGKAPNLLIYTASTLQSGVPSRFSGSGSGTDFTLTISSLQPEDFATYYC
QQANIFPLTFGGGTKVEIKGGGGSGGGGSGGGGSGGGGSQVQLVQSGAEVKKPGASVKVSCKASGYTFT
GYYMHWVRQAPGQGLEWMGWINPDSGGTNYAQKFQGRVTMTRDTSISTAYMELNRLRSDDTAVYYCARD
QPLGYCTNGVCSYFDYWGQGTLVTVSS
Second polypeptide (SEQ ID NO: 14):
same as the second polypeptide of Ly461
Ly739
First polypeptide (SEQ ID NO: 108):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSQVQLVQSGAEVKKPGASVK
VSCKASGYTFTGYYMHWVRQAPGQGLEWMGWINPDSGGTNYAQKFQGRVTMTRDTSISTAYMELNRLRS
DDTAVYYCARDQPLGYCTNGVCSYFDYWGQGTLVTVSSGGGGSGGGGSGGGGSGGGGSDIQMTQSPSSV
SASVGDRVTITCRASQGIYSWLAWYQQKPGKAPNLLIYTASTLQSGVPSRFSGSGSGTDFTLTISSLQP
EDFATYYCQQANIFPLTFGGGTKVEIK
Second polypeptide (SEQ ID NO: 14):
same as the second polypeptide of Ly461
Ly740
First polypeptide: (SEQ ID NO: 88):
same as the first polypeptide of Ly748
Second polypeptide (SEQ ID NO: 109):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSDIQMTQSPSSVSASVGDRVTITCRASQGIYSWLAWYQQKPGK
APNLLIYTASTLQSGVPSRFSGSGSGTDFTLTISSLQPEDFATYYCQQANIFPLTFGGGTKVEIKGGGG
SGGGGSGGGGSGGGGSQVQLVQSGAEVKKPGASVKVSCKASGYTFTGYYMHWVRQAPGQGLEWMGWINP
DSGGTNYAQKFQGRVTMTRDTSISTAYMELNRLRSDDTAVYYCARDQPLGYCTNGVCSYFDYWGQGTLV
TVSS
Ly741
First polypeptide: (SEQ ID NO: 88):
same as the first polypeptide of Ly740
Second polypeptide (SEQ ID NO: 110):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSQVQLVQSGAEVKKPGASVKVSCKASGYTFTGYYMHWVRQAPG
QGLEWMGWINPDSGGTNYAQKFQGRVTMTRDTSISTAYMELNRLRSDDTAVYYCARDQPLGYCTNGVCS
YFDYWGQGTLVTVSSGGGGSGGGGSGGGGSGGGGSDIQMTQSPSSVSASVGDRVTITCRASQGIYSWLA
WYQQKPGKAPNLLIYTASTLQSGVPSRFSGSGSGTDFTLTISSLQPEDFATYYCQQANIFPLTFGGGTK
VEIK
Ly742
First polypeptide (SEQ ID NO: 111):
QVQLVQSGAEVKKPGASVKVSCKASGYTFTGYYMHWVRQAPGQGLEWMGWINPDSGGTNYAQKFQGRVT
MTRDTSISTAYMELNRLRSDDTAVYYCARDQPLGYCTNGVCSYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGGGGGSGGGGSGGGGSGGGGSDIQMTQSPS
SLSASVGDRVTITCRASQDIRSNLNWYQQKPGGAVKLLIYYTSRLHSGVPSRFSGSGSGTDYTLTISSL
QPEDFATYFCQQSEKLPRTFGGGTKVEIRGGGGSGGGGSGGGGSGGGGSQVQLVQSGAEVKKPGASVKV
SCKASGYTFAGFEMHWVRQAPGQGLEWMGAIDPKTGGTDYNQKFKDRVTMTRDTSISTAYMELSRLRSD
DTAVYYCARDLGYFDVWGQGTLVTVSS
Second polypeptide (SEQ ID NO: 112)
DIQMTQSPSSVSASVGDRVTITCRASQGIYSWLAWYQQKPGKAPNLLIYTASTLQSGVPSRFSGSGSGT
DFTLTISSLQPEDFATYYCQQANIFPLTFGGGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGEC
Ly743
First polypeptide (SEQ ID NO: 113):
QVQLVQSGAEVKKPGASVKVSCKASGYTFTGYYMHWVRQAPGQGLEWMGWINPDSGGTNYAQKFQGRVT
MTRDTSISTAYMELNRLRSDDTAVYYCARDQPLGYCTNGVCSYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGGGGGSGGGGSGGGGSGGGGSQVQLVQSGA
EVKKPGASVKVSCKASGYTFAGFEMHWVRQAPGQGLEWMGAIDPKTGGTDYNQKFKDRVTMTRDTSIST
AYMELSRLRSDDTAVYYCARDLGYFDVWGQGTLVTVSSGGGGSGGGGSGGGGSGGGGSDIQMTQSPSSL
SASVGDRVTITCRASQDIRSNLNWYQQKPGGAVKLLIYYTSRLHSGVPSRFSGSGSGTDYTLTISSLQP
EDFATYFCQQSEKLPRTFGGGTKVEIR
Second polypeptide (SEQ ID NO: 112):
same as the second polypeptide of Ly742
Ly744
First polypeptide: (SEQ ID NO: 114):
QVQLVQSGAEVKKPGASVKVSCKASGYTFTGYYMHWVRQAPGQGLEWMGWINPDSGGTNYAQKFQGRVT
MTRDTSISTAYMELNRLRSDDTAVYYCARDQPLGYCTNGVCSYFDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Second polypeptide (SEQ ID NO: 115):
DIQMTQSPSSVSASVGDRVTITCRASQGIYSWLAWYQQKPGKAPNLLIYTASTLQSGVPSRFSGSGSGT
DFTLTISSLQPEDFATYYCQQANIFPLTFGGGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSDIQMTQSPSSLSASVGDRVTITCRASQDIRSNLNWYQQKPGG
AVKLLIYYTSRLHSGVPSRFSGSGSGTDYTLTISSLQPEDFATYFCQQSEKLPRTFGGGTKVEIRGGGG
SGGGGSGGGGSGGGGSQVQLVQSGAEVKKPGASVKVSCKASGYTFAGFEMHWVRQAPGQGLEWMGAIDP
KTGGTDYNQKFKDRVTMTRDTSISTAYMELSRLRSDDTAVYYCARDLGYFDVWGQGTLVTVSS
Ly745
First polypeptide (SEQ ID NO: 114):
same as the first polypeptide of Ly744
Second polypeptide (SEQ ID NO: 116):
DIQMTQSPSSVSASVGDRVTITCRASQGIYSWLAWYQQKPGKAPNLLIYTASTLQSGVPSRFSGSGSGT
DFTLTISSLQPEDFATYYCQQANIFPLTFGGGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSQVQLVQSGAEVKKPGASVKVSCKASGYTFAGFEMHWVRQAPG
QGLEWMGAIDPKTGGTDYNQKFKDRVTMTRDTSISTAYMELSRLRSDDTAVYYCARDLGYFDVWGQGTL
VTVSSGGGGSGGGGSGGGGSGGGGSDIQMTQSPSSLSASVGDRVTITCRASQDIRSNLNWYQQKPGGAV
KLLIYYTSRLHSGVPSRFSGSGSGTDYTLTISSLQPEDFATYFCQQSEKLPRTFGGGTKVEIR

### Characterization of Anti-CD40/CD137 Bi-Specific Antibodies

(i) Binding Activity

Anti-CD40/CD137 bi-specific antibodies were analyzed by FACS for their binding properties to human CD40 and/or human CD137 expressed on CHO cells. Briefly, cultured cells were harvested, counted and cell viability was evaluated using the Trypan Blue exclusion method. Viable cells were then adjusted to 2×10⁶ cells per mL in PBS containing 2% BSA. 100 μL of this cell suspension were further aliquoted per well into a V-bottom 96-well plate. 50 μL of the bi-specific antibodies or corresponding IgG control were added to the cell-containing wells to obtain final concentrations of 0.1 μg/mL to 10 μg/mL. After incubation for 2 hours at 4° C., cells were centrifuged (3 min, 1000×g), washed with 250 μL/well BSA-containing FACS Stain Buffer, resuspended and incubated for an additional 1 hour at 4° C. with 100 μL/well fluorochrome-conjugated anti-IgG antibody for detection of the bisepecific antibody. Cells were then washed with 250 μL/well BSA-containing FACS Stain Buffer, resuspended in 100 μL/well FACS Stain Buffer, acquired and analyzed using a FACS machine. Binding of the bispecific antibodies to human CD40 or human CD137 expressing CHO cells were evaluated and the mean fluorescence intensity is plotted in histograms or dot plots.

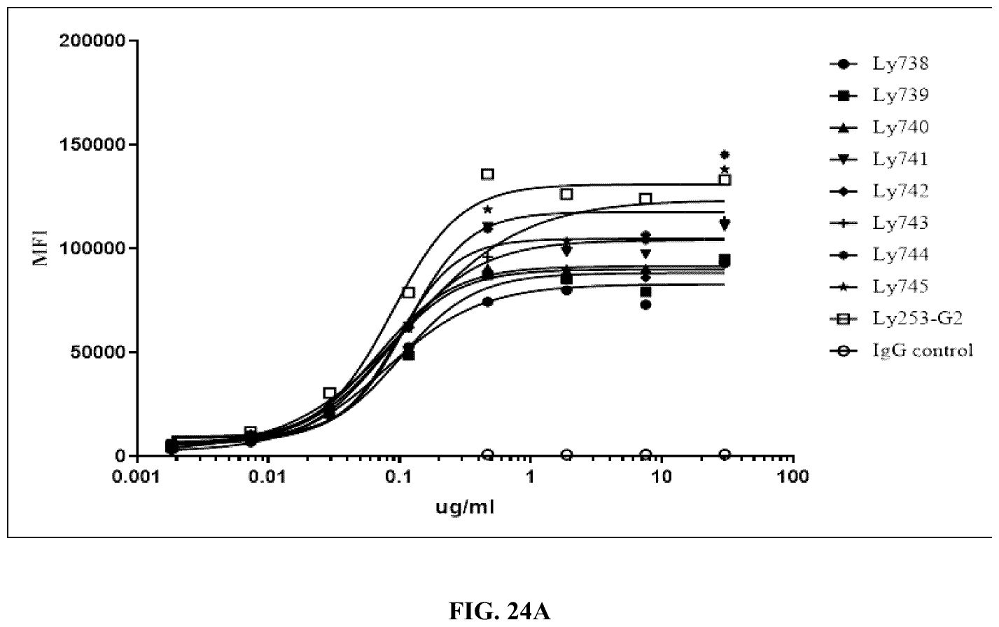

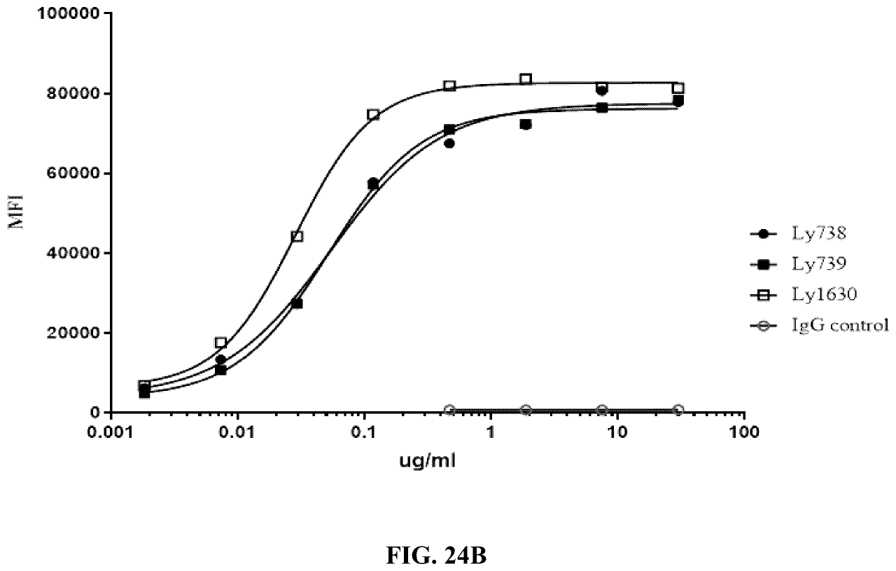

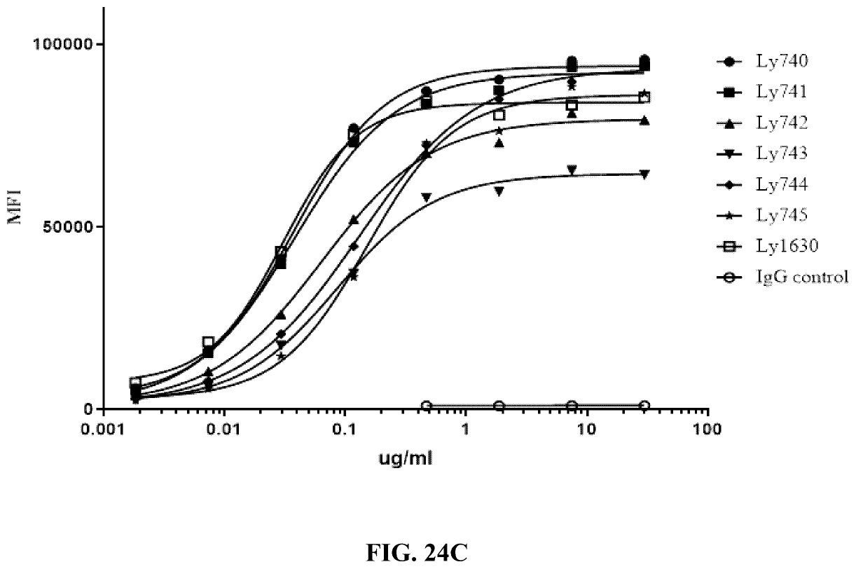

As shown in FIG. 24A, the exemplary anti-CD40/CD137 bi-specific antibodies exhibited similar binding affinity to human CD40 expressed on the CHO cells over-expressing such as their parental mAb Ly253-G2. As shown in FIGS. 24B and 24C, the bi-specific antibodies exhibited binding affinity to human CD137 expressed on CHO cells. Compared to the corresponding parental antibody, the binding activity of bi-specific antibodies remain minimally changed.

The bispecific antibodies are evaluated for their in vitro and in vivo activity, including agonistic activity in the CD40 and CD137 reporter assay systems, co-stimulation assays, and anti-tumor activity in mouse models.

### Example 5: Bi-Specific Antibodies to OX40 and CD137

### Preparation of Anti-OX40/CD137 Bi-Specific Antibodies

Anti-OX40/CD137 bi-specific antibodies were produced using the anti-CD137 antibody Ly1630 and anti-OX40 Ly598. The structural information of Ly1630 is provided in Example 1 above. The amino acid sequences of Ly598 are provided below

Ly598
Heavy chain
(SEQ ID NO: 117)
EVQLVQSGAEVKKPGASVKVSCKASGYTFTDSYMSWVRQAPGQGLEW
IGDMYPDNGDSSYNQKFRERVTITRDTSTSTAYLELSSLRSEDTAVY
YCVLAPRWYFSVWGQGTLVTVSSASTKGPSVFPLAPSSKSTSGGTAA
LGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALP
APIEKTISKAKGQPREPQVYTLPPSREEMTKNQVSLTCLVKGFYPSD
IAVEWESNGQPENNYKTTPPVLDSDGSFFLYSKLTVDKSRWQQGNVF
SCSVMHEALHNHYTQKSLSLSPGK
Light chain
(SEQ ID NO: 118)
DIQMTQSPSSLSASVGDRVTITCRASQDISNYLNWYQQKPGKAPKLL
IYYTSRLRSGVPSRFSGSGSGTDFTLTISSLQPEDFATYYCQQGHTL
PPTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

cDNAs encoding the V_{H} and V_{L} chains of the parent anti-CD137 and anti-OX40 antibodies were used as the starting materials for making the bi-specific antibodies. CHO-cell transient expression was carried out with plasmids configured for expressing polypeptide chains of the bi-specific antibodies. These antibodies were purified by protein A affinity chromatography.

The amino acid sequences of the polypeptides of the bi-specific antibodies are provided below:

Ly762
First polypeptide (SEQ ID NO: 119):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSDIQMTQSPSSLSASVGDRV
TITCRASQDISNYLNWYQQKPGKAPKLLIYYTSRLRSGVPSRFSGSGSGTDFTLTISSLQPEDFATYYC
QQGHTLPPTFGQGTKVEIKGGGGSGGGGSGGGGSGGGGSEVQLVQSGAEVKKPGASVKVSCKASGYTFT
DSYMSWVRQAPGQGLEWIGDMYPDNGDSSYNQKFRERVTITRDTSTSTAYLELSSLRSEDTAVYYCVLA
PRWYFSVWGQGTLVTVSS
Second polypeptide (SEQ ID NO: 14):
same as the second chain of Ly461
Ly763
First polypeptide (SEQ ID NO: 120):
QVQLVQSGAEVKKPGASVKVSCKASGYTFAGFEMHWVRQAPGQGLEWMGAIDPKTGGTDYNQKFKDRVT
MTRDTSISTAYMELSRLRSDDTAVYYCARDLGYFDVWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGGGGGSGGGGSGGGGSGGGGSEVQLVQSGAEVKKPGASVK
VSCKASGYTFTDSYMSWVRQAPGQGLEWIGDMYPDNGDSSYNQKFRERVTITRDTSTSTAYLELSSLRS
EDTAVYYCVLAPRWYFSVWGQGTLVTVSSGGGGSGGGGSGGGGSGGGGSDIQMTQSPSSLSASVGDRVT
ITCRASQDISNYLNWYQQKPGKAPKLLIYYTSRLRSGVPSRFSGSGSGTDFTLTISSLQPEDFATYYCQ
QGHTLPPTFGQGTKVEIK
Second polypeptide (SEQ ID NO: 14):
same as the light chain of Ly461
Ly764
First polypeptide: (SEQ ID NO: 88):
same as the first polypeptide of Ly748
Second polypeptide (SEQ ID NO: 121):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSDIQMTQSPSSLSASVGDRVTITCRASQDISNYLNWYQQKPGK
APKLLIYYTSRLRSGVPSRFSGSGSGTDFTLTISSLQPEDFATYYCQQGHTLPPTFGQGTKVEIKGGGG
SGGGGSGGGGSGGGGSEVQLVQSGAEVKKPGASVKVSCKASGYTFTDSYMSWVRQAPGQGLEWIGDMYP
DNGDSSYNQKFRERVTITRDTSTSTAYLELSSLRSEDTAVYYCVLAPRWYFSVWGQGTLVTVSS
Ly765
First polypeptide (SEQ ID NO: 88):
same as the first polypeptide of Ly748
Second polypeptide (SEQ ID NO: 122):
DIQMTQSPSSLSASVGDRVTITCRASQDIRSNLNWYQQKPGGAVKLLIYYTSRLHSGVPSRFSGSGSGT
DYTLTISSLQPEDFATYFCQQSEKLPRTFGGGTKVEIRRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSEVQLVQSGAEVKKPGASVKVSCKASGYTFTDSYMSWVRQAPG
QGLEWIGDMYPDNGDSSYNQKFRERVTITRDTSTSTAYLELSSLRSEDTAVYYCVLAPRWYFSVWGQGT
LVTVSSGGGGSGGGGSGGGGSGGGGSDIQMTQSPSSLSASVGDRVTITCRASQDISNYLNWYQQKPGKA
PKLLIYYTSRLRSGVPSRFSGSGSGTDFTLTISSLQPEDFATYYCQQGHTLPPTFGQGTKVEIK
Ly766
First polypeptide (SEQ ID NO: 123):
EVQLVQSGAEVKKPGASVKVSCKASGYTFTDSYMSWVRQAPGQGLEWIGDMYPDNGDSSYNQKFRERVT
ITRDTSTSTAYLELSSLRSEDTAVYYCVLAPRWYFSVWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGGGGGSGGGGSGGGGSGGGGSDIQMTQSPSSLSASVGDR
VTITCRASQDIRSNLNWYQQKPGGAVKLLIYYTSRLHSGVPSRFSGSGSGTDYTLTISSLQPEDFATYF
CQQSEKLPRTFGGGTKVEIRGGGGSGGGGSGGGGSGGGGSQVQLVQSGAEVKKPGASVKVSCKASGYTF
AGFEMHWVRQAPGQGLEWMGAIDPKTGGTDYNQKFKDRVTMTRDTSISTAYMELSRLRSDDTAVYYCAR
DLGYFDVWGQGTLVTVSS
Second polypeptide: (SEQ ID NO: 118):
same as the light chain of Ly598
Ly767
First polypeptide (SEQ ID NO: 136):
EVQLVQSGAEVKKPGASVKVSCKASGYTFTDSYMSWVRQAPGQGLEWIGDMYPDNGDSSYNQKFRERVT
ITRDTSTSTAYLELSSLRSEDTAVYYCVLAPRWYFSVWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGGGGGSGGGGSGGGGSGGGGSQVQLVQSGAEVKKPGASV
KVSCKASGYTFAGFEMHWVRQAPGQGLEWMGAIDPKTGGTDYNQKFKDRVTMTRDTSISTAYMELSRLR
SDDTAVYYCARDLGYFDVWGQGTLVTVSSGGGGSGGGGSGGGGSGGGGSDIQMTQSPSSLSASVGDRVT
ITCRASQDIRSNLNWYQQKPGGAVKLLIYYTSRLHSGVPSRFSGSGSGTDYTLTISSLQPEDFATYFCQ
QSEKLPRTFGGGTKVEIR
Second polypeptide(SEQ ID NO: 118):
same as the light chain of Ly598
Ly768
First polypeptide: (SEQ ID NO: 125):
EVQLVQSGAEVKKPGASVKVSCKASGYTFTDSYMSWVRQAPGQGLEWIGDMYPDNGDSSYNQKFRERVT
ITRDTSTSTAYLELSSLRSEDTAVYYCVLAPRWYFSVWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
Second polypeptide (SEQ ID NO: 126):
DIQMTQSPSSLSASVGDRVTITCRASQDISNYLNWYQQKPGKAPKLLIYYTSRLRSGVPSRFSGSGSGT
DFTLTISSLQPEDFATYYCQQGHTLPPTFGQGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSDIQMTQSPSSLSASVGDRVTITCRASQDIRSNLNWYQQKPGG
AVKLLIYYTSRLHSGVPSRFSGSGSGTDYTLTISSLQPEDFATYFCQQSEKLPRTFGGGTKVEIRGGGG
SGGGGSGGGGSGGGGSQVQLVQSGAEVKKPGASVKVSCKASGYTFAGFEMHWVRQAPGQGLEWMGAIDP
KTGGTDYNQKFKDRVTMTRDTSISTAYMELSRLRSDDTAVYYCARDLGYFDVWGQGTLVTVSS
Ly769
First polypeptide (SEQ ID NO: 125):
same as the first polypeptide of Ly768
Second polypeptide (SEQ ID NO: 127):
DIQMTQSPSSLSASVGDRVTITCRASQDISNYLNWYQQKPGKAPKLLIYYTSRLRSGVPSRFSGSGSGT
DFTLTISSLQPEDFATYYCQQGHTLPPTFGQGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECGGGGSGGGGSGGGGSGGGGSQVQLVQSGAEVKKPGASVKVSCKASGYTFAGFEMHWVRQAPG
QGLEWMGAIDPKTGGTDYNQKFKDRVTMTRDTSISTAYMELSRLRSDDTAVYYCARDLGYFDVWGQGTL
VTVSSGGGGSGGGGSGGGGSGGGGSDIQMTQSPSSLSASVGDRVTITCRASQDIRSNLNWYQQKPGGAV
KLLIYYTSRLHSGVPSRFSGSGSGTDYTLTISSLQPEDFATYFCQQSEKLPRTFGGGTKVEIR

### Characterization of Anti-OX40/CD137 Bi-Specific Antibodies

(i) Binding Activity

Anti-OX40/CD137 bi-specific antibodies were analyzed by FACS for their binding properties to human OX40 and/or human CD137 expressed on CHO cells. Briefly, cultured cells were harvested, counted and cell viability was evaluated using the Trypan Blue exclusion method. Viable cells were then adjusted to 2×10⁶ cells per mL in PBS containing 2% BSA. 100 μL of this cell suspension were further aliquoted per well into a V-bottom 96-well plate. 50 μL of the bi-specific antibodies or corresponding IgG control were added to the cell-containing wells to obtain final concentrations of 0.1 μg/mL to 10 μg/mL. After incubation for 2 hours at 4° C., cells were centrifuged (3 min, 1000×g), washed with 250 μL/well BSA-containing FACS Stain Buffer, resuspended and incubated for an additional 1 hour at 4° C. with 100 μL/well fluorochrome-conjugated anti-IgG antibody for detection of the bisepecific antibody. Cells were then washed with 250 μL/well BSA-containing FACS Stain Buffer, resuspended in 100 μL/well FACS Stain Buffer, acquired and analyzed using a FACS machine. Binding of the bispecific antibodies to human OX40 or human CD137 expressing CHO cells were evaluated and the mean fluorescence intensity is plotted in histograms or dot plots.

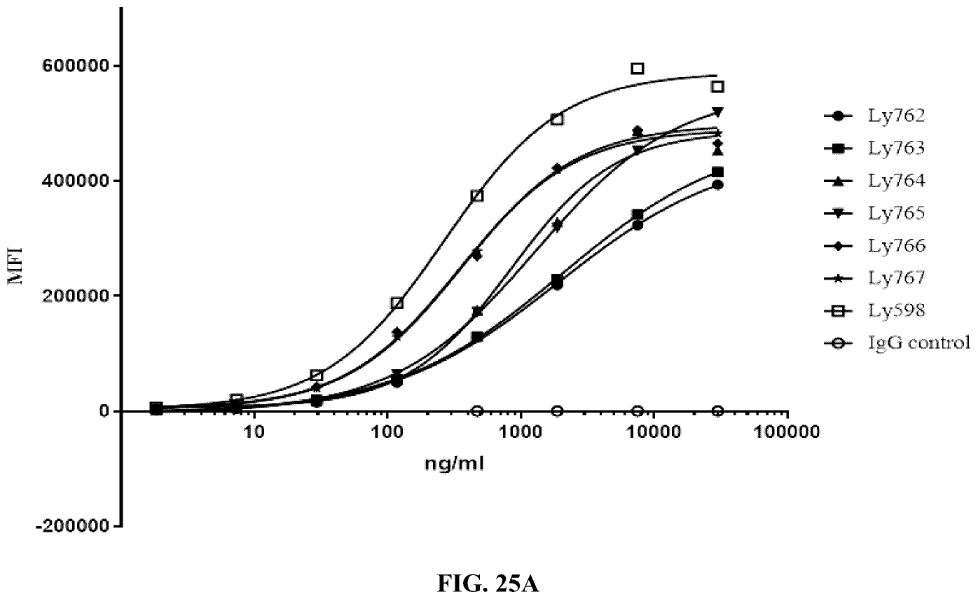

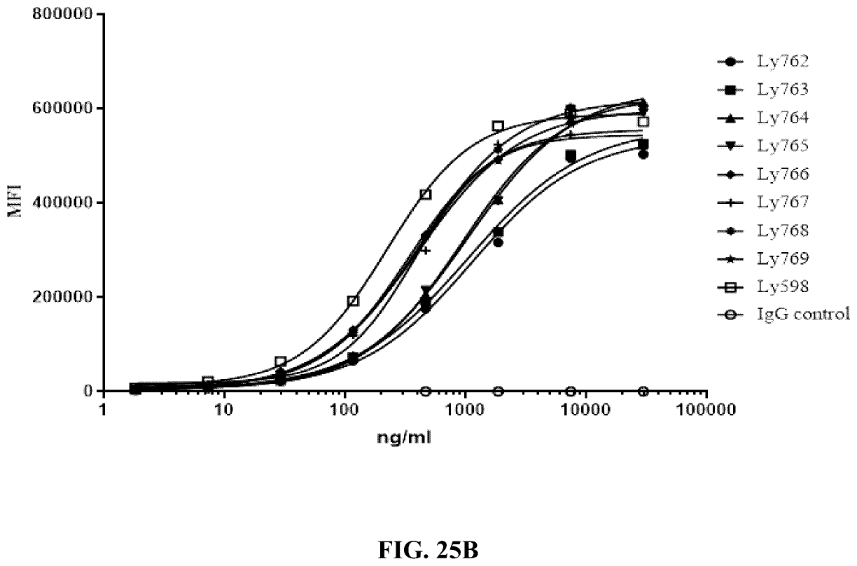

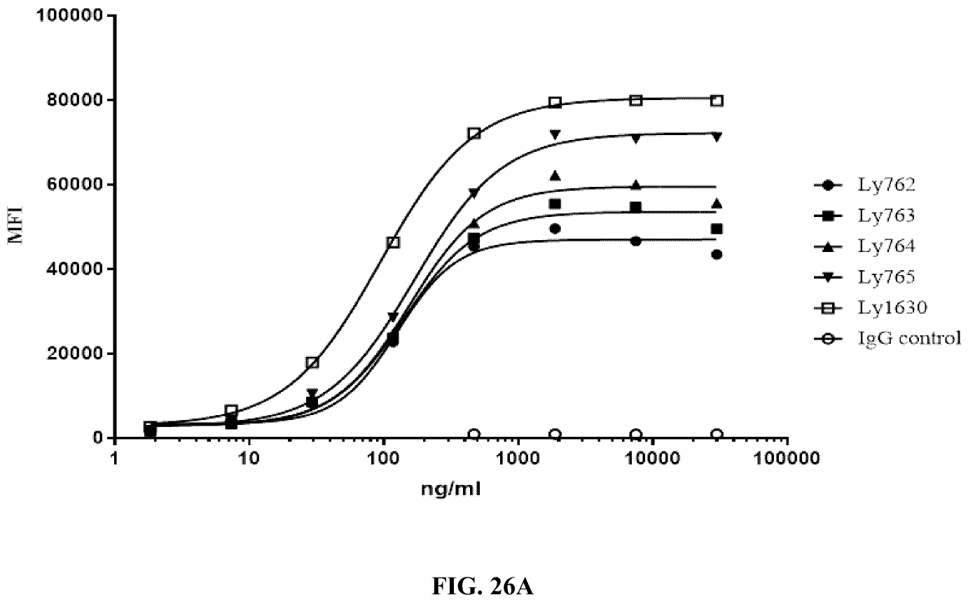

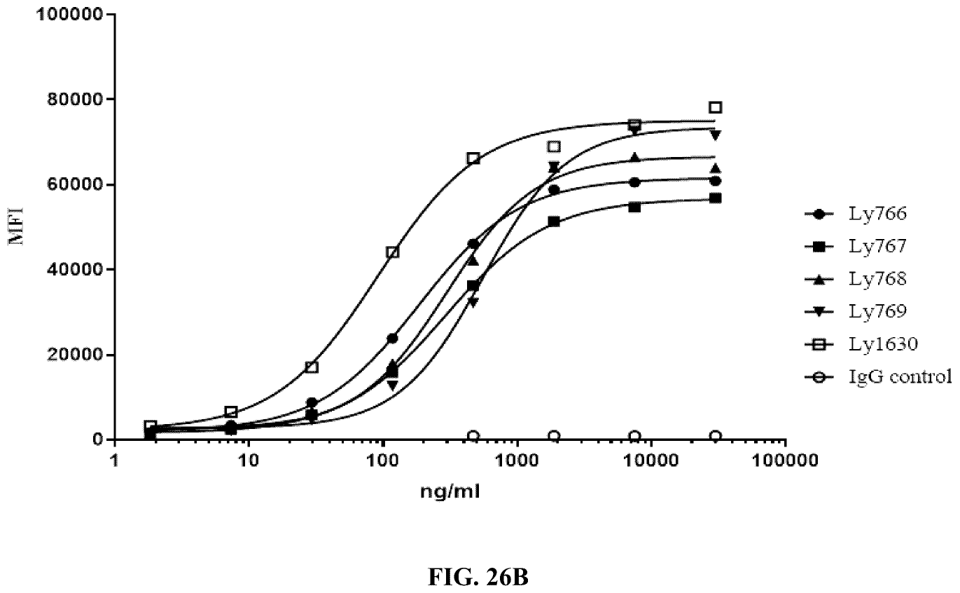

The exemplary anti-OX40/CD137 bi-specific antibodies exhibited similar or reduced binding affinity to human OX40 (FIGS. 25A and 25B) or human CD137 (FIGS. 26A and 26B) expressed on the CHO cells, as compared with their parental mAb clones.

(ii) Agonistic Activity for CD137

To determine the agonist activity of these anti-OX40/CD137 bi-specific antibodies, a CD137 reporter assay was developed, which involves reporter cells over-expressing human CD137. The CD137 reporter assay was performed in co-culture with OX40-expressing CHO cells following the procedures as described in Example 1.

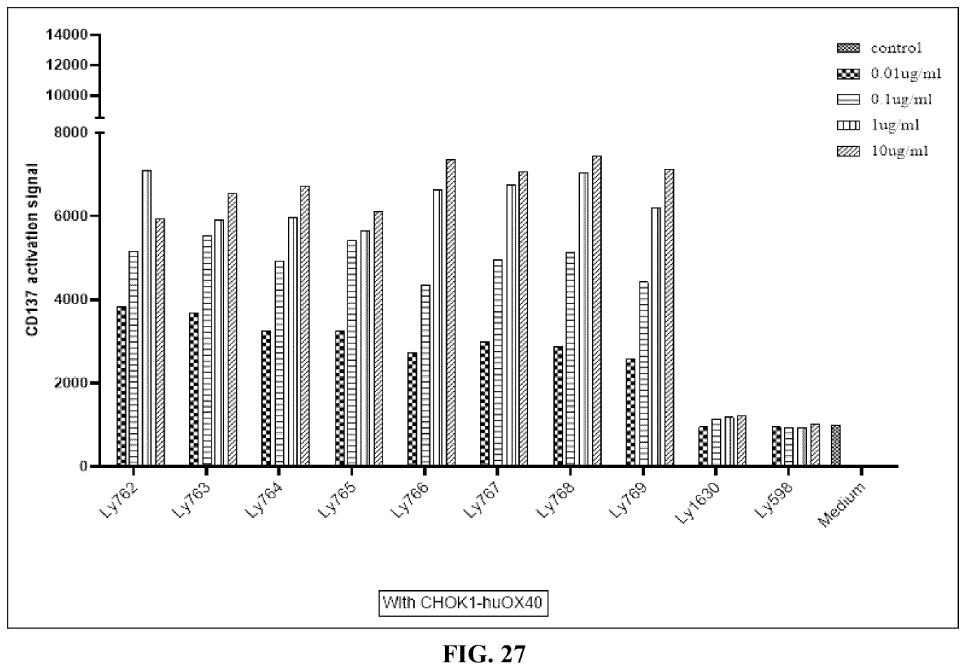

As shown in FIG. 27, the agonist activity of exemplary bi-specific antibodies was greatly enhanced in the co-culture assay, as compared with their parental mAbs, which didn't show any agonist activity. Binding to both CD137 and OX40 by the tested exemplary bi-specific antibodies simultaneously in a microenvironment would affect individual binding due to at least the avidity effect. The bi-specific antibodies showed increased activity dependent on their binding activity to OX40. Therefore, binding profile to human OX40 and CD137 would affect the agonist activity of these bi-specific antibodies.

The bispecific antibodies are evaluated for their agonistic activity in a OX40 reporter assay system.

(iii) PBMC Activation

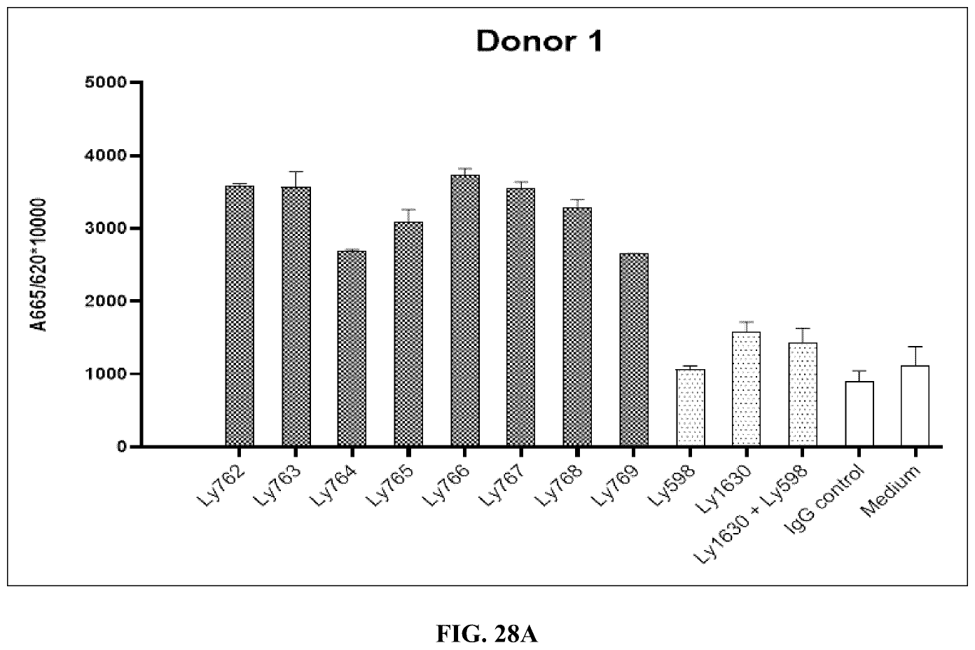

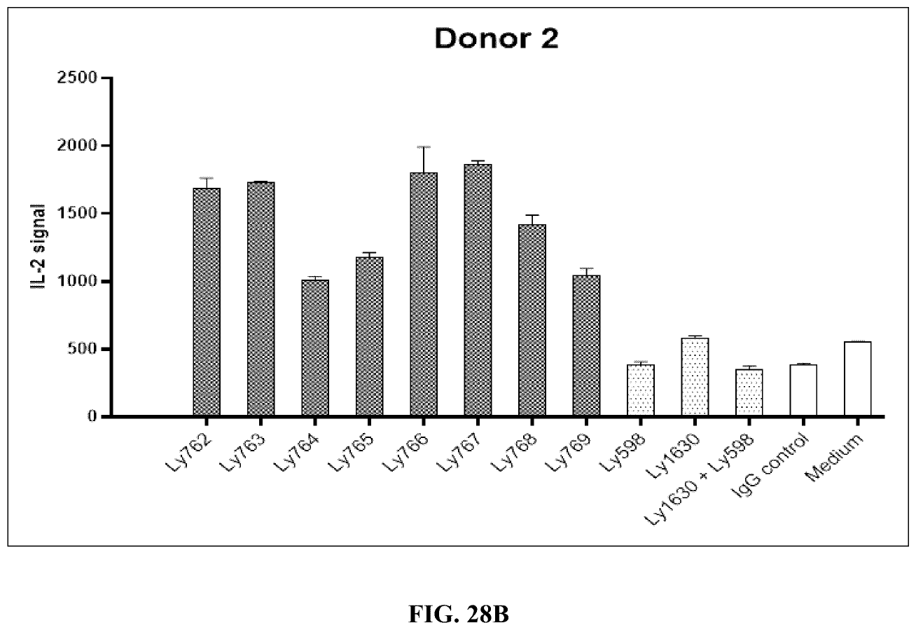

A PBMC activation assay was performed to show the co-stimulation functionality of the bispecific antibodies. Briefly, 2×10⁵ PBMCs in 100 μL culture medium added SEB (final concentration at 0.01 μg/mL) and serial diluted antibody samples in 100 μL culture medium were added to plates and incubated at 37° C. with 5% CO₂ for 5 days. Cell culture supernatants were collected for cytokine detection after 5 days stimulation using Human IL-2 detection kit (Ref) following the instruction manual. FIGS. 28A and 28B shows that the exemplary bispecific antibodies induced stronger IL-2 production from human PBMCs than anti-OX40 or anti-CD137 mAb clones alone or in combination. Therefore, binding to CD137 and OX40 by antibody molecules simultaneously in a microenvironment would enhance PBMC stimulation activity of these bi-specific antibodies compared with their parental mAbs.

(iv) Pharmacokinetic Studies of Anti-OX40/CD137 Bi-Specific Antibodies

C57BL/6 mice (6-7 weeks old, 19-20 g, female, purchased from Vital River) were used for the study. Antibodies were formulated in PBS and administered via tail vein injection at 5 mg/kg in a group of 4 mice.

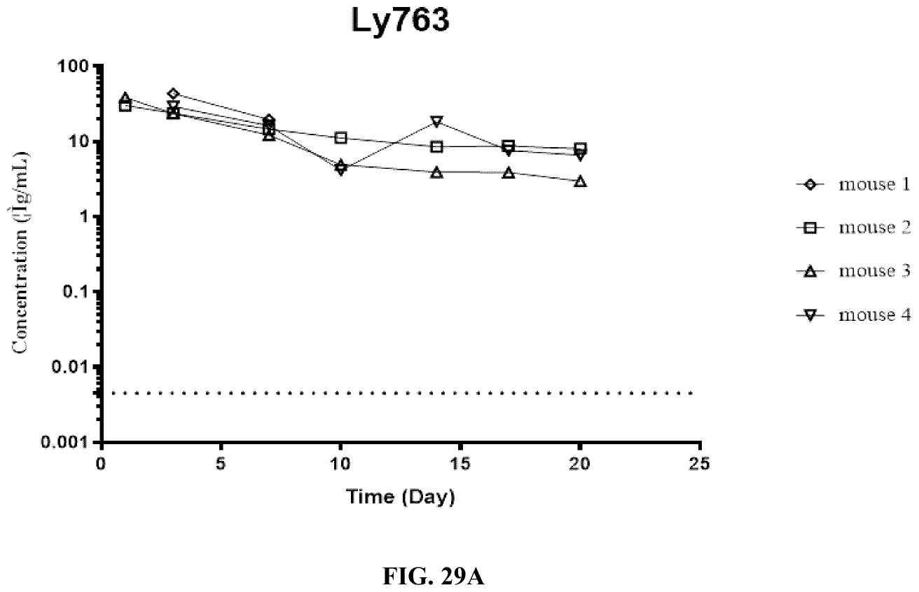

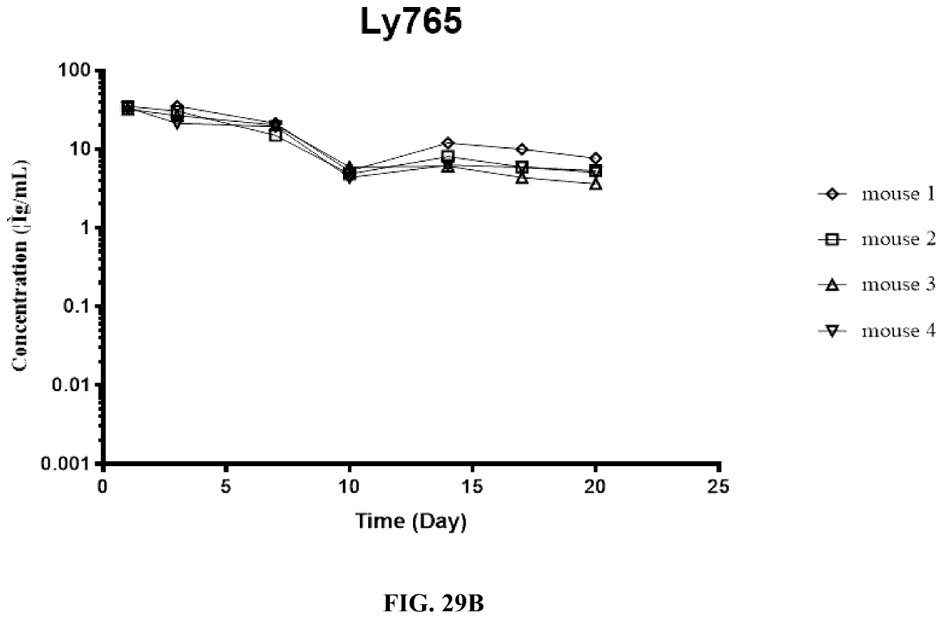

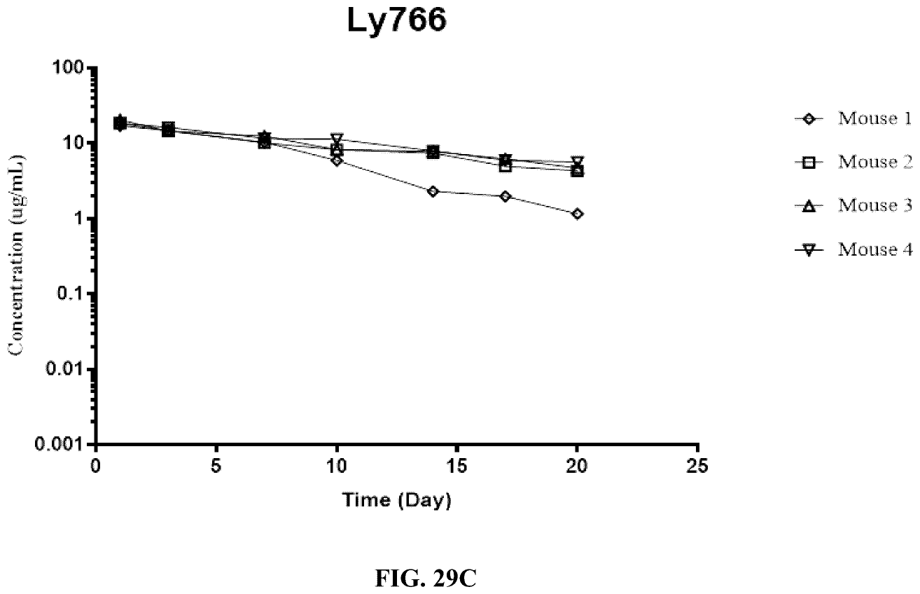

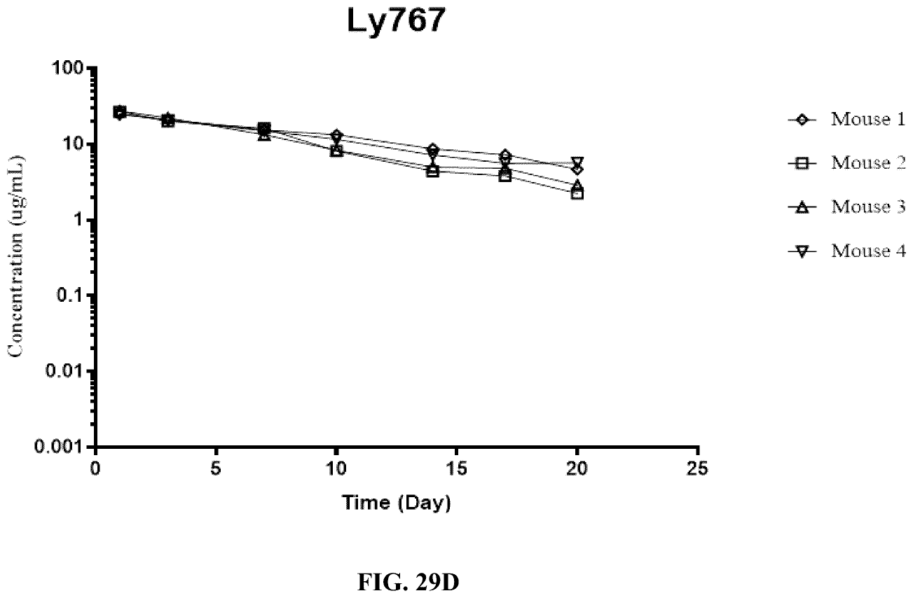

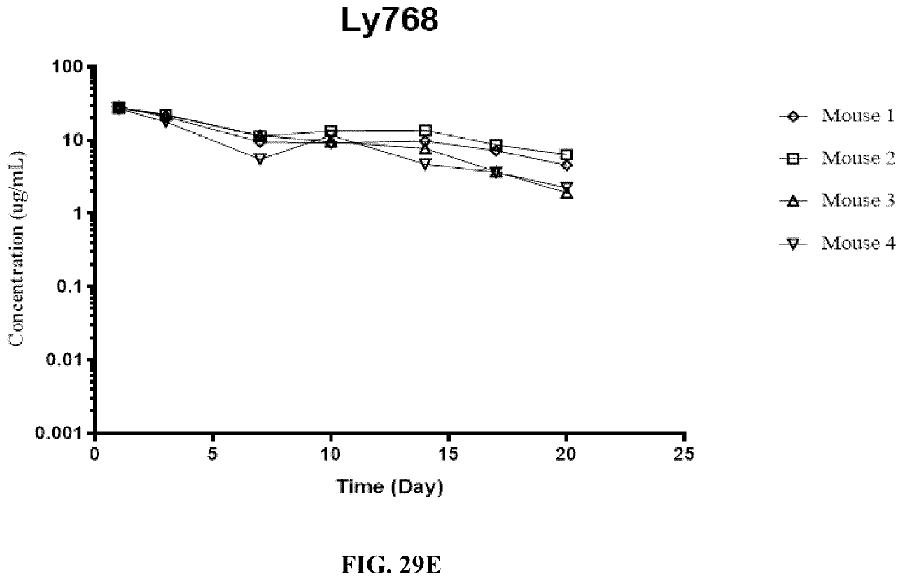

Blood sampling was done at pre-dose, Id, 4d, 7d, 10d, 14d, 17d and 21d by serial bleeding. 10 uL blood per time point was added to 40 uL of a PBS-BSA solution. The sample was then mixed well and centrifuged at 2000 g for 5 minutes at 4° C. The supernatant was put on dry ice immediately after collection and stored at approximately −70° C. until analysis. Blood antibody concentrations were determined by bridge ELISA to detect simultaneous CD137 and OX40 binding. FIGS. 29A-29E showed the blood concentrations of the bispecific antibodies after a single intravenous injection of 5 mg/kg. These bispecific antibodies showed high and lasting circulation concentrations.

The bispecific antibodies are evaluated for their in vitro and in vivo activity, including agonistic activity in co-stimulation assays and anti-tumor activity in mouse models.

(v) Anti-Tumor Activity

Exemplary anti-OX40/CD137 antibodies were tested in vivo to determine their anti-tumor efficacy and toxicity. Briefly, human PBMCs were collected from healthy volunteers and were injected with human melanoma A375 cells into mice subcutaneously. Mice were grouped by body weight the day PBMC and A375 cells were inoculated. Anti-GITR/CD137 antibodies were administered by intraperitoneal injections and tumor sizes were measure during 3-4 weeks of antibody treatment. Tumor sizes were calculated as tumor volume using formula of 0.5×length×width²

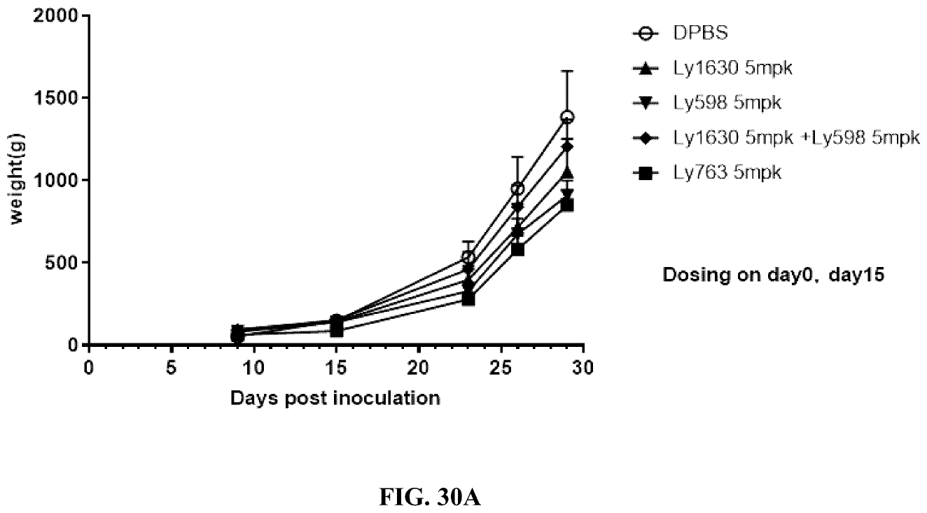

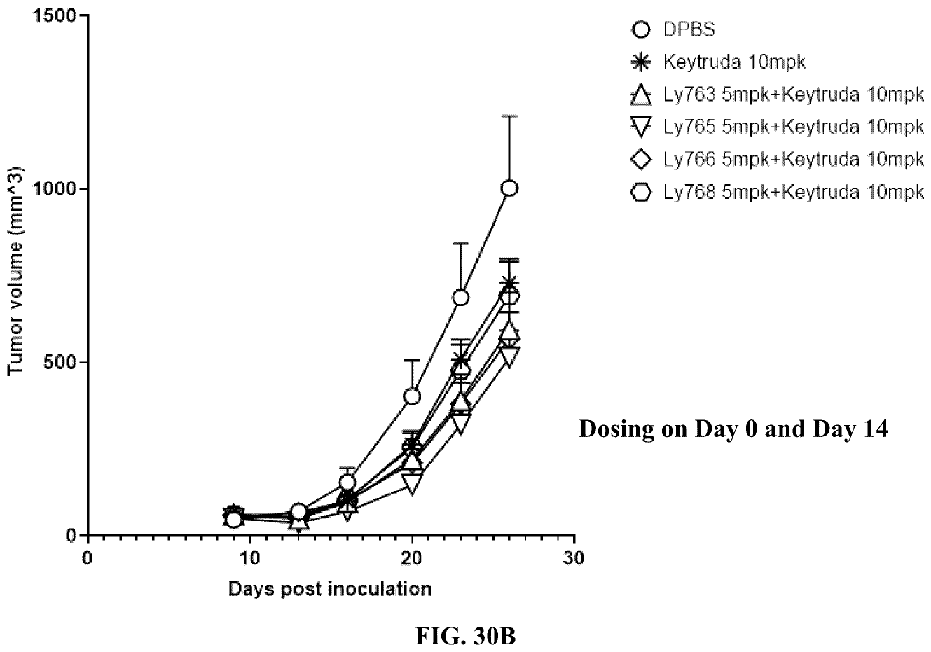

Anti-tumor efficacy was evaluated between tumor sizes of the control group and antibody treatment group as shown in FIGS. 30A and 30B. Ly763 exhibited stronger inhibition of tumor growth than PD-1 antibody Keytruda®. The combination of Ly763 or Ly765 and Keytruda® exhibited stronger inhibition of tumor growth than Keytruda® monotherapy

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A bi-specific antibody, comprising:
(a) a first antibody moiety that binds human CD137, and
(b) a second antibody moiety that binds an antigen selected from the group consisting of PD-1, PD-L1, GITR, CD40 and OX40.

2. The bi-specific antibody of claim 1, wherein either the first antibody moiety or the second antibody moiety is in a single-chain antibody (scFv) format.

3. The bi-specific antibody of claim 2, wherein the other antibody moiety is in a full-length antibody format comprising a heavy chain and a light chain.

4. The bi-specific antibody of claim 2 or claim 3, wherein the first antibody moiety that binds human CD137 is a scFv; wherein the second antibody moiety that binds PD-1, PD-L1, GITR, CD40 or OX40 comprises a first polypeptide comprising an antibody heavy chain and a second polypeptide comprising an antibody light chain, and wherein the scFv is fused to either the first polypeptide or the second polypeptide.

5. The bi-specific antibody of claim 2 or claim 3, wherein the second antibody moiety that binds PD-1, PD-L1, GITR, CD40 or OX40 is a scFv; wherein the first antibody moiety that binds human CD137 comprises a first polypeptide comprising an antibody heavy chain and a second polypeptide comprising an antibody light chain, and wherein the scFv is fused to either the first polypeptide or the second polypeptide.

6. The bi-specific antibody of claim 1, wherein the bi-specific antibody comprises:
(i) a first polypeptide, which comprises a heavy chain of the first antibody moiety fused to a light chain of the second antibody moiety;
(ii) a second polypeptide, which comprises a light chain of the first antibody moiety; and
(iii) a third polypeptide, which comprises a heavy chain of the second antibody moiety.

7. The bi-specific antibody of claim 1, wherein the bi-specific antibody comprises:
(i) a first polypeptide, which comprises a heavy chain of the second antibody moiety fused to a light chain of the first antibody moiety;
(ii) a second polypeptide, which comprises a light chain of the second antibody moiety; and
(iii) a third polypeptide, which comprises a heavy chain of the first antibody moiety.

8. The bi-specific antibody of claim 6 or claim 7, wherein in (iii), the heavy chain of the second antibody moiety or the first antibody moiety comprises a V_{H} and a heavy chain constant domain, which optionally is CH1.

9. The bi-specific antibody of any one of claims 1-8, wherein the first antibody moiety that binds human CD137 has the same heavy chain and light chain CDRs as reference antibody Ly1630, optionally wherein the first antibody moiety that binds human CD137 has the same V_{H} and/or V_{L} as reference antibody Ly1630.

10. The bi-specific antibody of any one of claims 1-9, wherein the second antibody moiety binds PD-1.

11. The bi-specific antibody of claim 10, wherein the second antibody moiety comprises the same heavy chain CDRs as reference antibody Ly516, and/or wherein the second antibody moiety comprises the same light chain CDRs as reference antibody Ly516, optionally wherein the second antibody moiety comprises the same V_{H} and/or V_{L} as reference antibody Ly516.

12. The bi-specific antibody of claim 11, which is selected from the group consisting of Ly456, Ly457, Ly458, Ly459, Ly460, Ly461, Ly510, Ly511, Ly512, Ly513, Ly514 and Ly515.

13. The bi-specific antibody of any one of claims 1-9, wherein the second antibody moiety binds PD-L1.

14. The bi-specific antibody of claim 9, wherein the second antibody moiety comprises the same heavy chain CDRs as reference antibody Ly076, and/or wherein the second antibody moiety comprises the same light chain CDRs as reference antibody Ly076, optionally wherein the second antibody moiety comprises the same V_{H} and/or V_{L} as reference antibody Ly076.

15. The bi-specific antibody of claim 14, which is selected from the group consisting of Ly299, Ly346, Ly347, and Ly348.

16. The bi-specific antibody of any one of claims 1-B, wherein the second antibody moiety binds GITR.

17. The bi-specific antibody of claim 16, wherein the second antibody moiety is an anti-GITR antibody set forth in any one of claims 26-39.

18. The bi-specific antibody of claim 17, which is selected from the group consisting of Ly746, Ly747, Ly748, Ly749, Ly750, Ly751, Ly752, Ly753, Ly754, Ly755, Ly756, Ly757, Ly758, Ly759, Ly760, Ly761, Ly1523, Ly1524, Ly1525, and Ly1526.

19. The bi-specific antibody of any one of claims 1-9, wherein the second antibody moiety binds CD40.

20. The bi-specific antibody of claim 19, wherein the second antibody moiety comprises the same heavy chain CDRs as reference antibody Ly253, and/or wherein the second antibody moiety comprises the same light chain CDRs as reference antibody Ly253, optionally wherein the second antibody moiety comprises the same V_{H} and/or V_{L} as reference antibody Ly253.

21. The bi-specific antibody of claim 20, which is selected from the group consisting of Ly738, Ly739, Ly740, Ly741, Ly742, Ly743, Ly744, and Ly745.

22. The bi-specific antibody of any one of claims 1-9, wherein the second antibody moiety binds OX40.

23. The bi-specific antibody of claim 22, wherein the second antibody moiety comprises the same heavy chain CDRs as reference antibody Ly598; and/or wherein the second antibody moiety comprises the same light chain CDRs as the reference antibody Ly598, and optionally wherein the second antibody moiety comprises the same V_{H} and/or V_{L} as reference antibody Ly598.

24. The bi-specific antibody of claim 23, which is selected from the group consisting of Ly762, Ly763, Ly764, Ly765, Ly766, Ly767, Ly768, Ly1519, Ly1520, Ly1521, and Ly1522.

25. The bi-specific antibody of claim 1, which is any of the bi-specific antibodies set forth in any one of Examples 1-5.

26. An isolated antibody specific to human glucocorticoid-induced TNFR-related protein (GITR) (anti-GITR antibody), wherein the anti-GITR antibody comprises:
(a) a heavy chain variable region (V_{H}) comprising heavy chain complementary determining regions (CDRs) 1, 2, and 3, which are either identical to those of a reference antibody, or contain no more than five amino acid residue variations relative to the reference antibody, wherein the reference antibody is Lyv392 or Lyv396; and
(b) a light chain variable region (V_{L}), comprising light chain complementary determining regions (CDRs) 1, 2, and 3, which are either identical to those of the reference antibody or contain no more than five amino acid residue variations relative to the reference antibody.

27. The isolated anti-GITR antibody of claim 26, wherein the anti-GITR antibody is a humanized antibody comprising a human V_{H} framework and a human V_{L} framework.

28. The isolated anti-GITR antibody of claim 27, wherein the human V_{H} framework region is from IGHV4-59*01, and/or wherein the human V_{L} framework is from IGKV3-11*01.

29. The isolated anti-GITR antibody of claim 27 or claim 28, wherein the V_{L} comprises one or more mutations in the human V_{L} framework.

30. The isolated anti-GITR antibody of claim 29, wherein the one or more mutations in the V_{L} framework are back mutations based on amino acid residues in the reference antibody Lyv392 at corresponding positions.

31. The anti-GITR antibody of claim 30, wherein the one or more back mutations comprise E1D, I2T, I48V, V85T, Y87F, or a combination thereof.

32. The anti-GITR antibody of claim 27, wherein the V_{L} comprises the amino acid sequence of SEQ ID NO:69, SEQ ID NO:72, or SEQ ID NO:81.

33. The isolated anti-GITR antibody of any one of claims 27-32, wherein the V_{H} comprises the amino acid sequence of SEQ ID NO:68 or SEQ ID NO:80.

34. The isolated anti-GITR antibody of claim 27, wherein the antibody comprises:
(a) a V_{H} chain comprising the amino acid sequence of SEQ ID NO:68 and a V_{L} chain comprising the amino acid sequence of SEQ ID NO:69;
(b) a V_{H} chain comprising the amino acid sequence of SEQ ID NO:68 and a V_{L} chain comprising the amino acid sequence of SEQ ID NO:72; or
(c) a V_{H} chain comprising the amino acid sequence of SEQ ID NO:80 and a V_{L} chain comprising the amino acid sequence of SEQ ID NO:81.

35. The humanized antibody of any one of claims 26-34, wherein the antibody is a full-length antibody.

36. The humanized antibody of claim 35, wherein the full-length antibody is an IgG/kappa molecule.

37. The humanized antibody of claim 36, wherein the full-length antibody comprises a heavy chain that is an IgG1, IgG2, or IgG4 chain.

38. The humanized antibody of claim 37, wherein the heavy chain comprises a mutated Fc region, which exhibits altered binding affinity or selectivity to an Fc receptor.

39. The humanized antibody of claim 38, wherein the antibody is selected from the group consisting of TM676, TM677, and TM685.

40. A nucleic acid or a nucleic acid set, which collectively encodes an antibody of any one of the preceding claims.

41. The nucleic acid or nucleic acid set of claim 40, which is an expression vector or an expression vector set.

42. A host cell, comprising the nucleic acid or nucleic acid set of claim 40 or claim 41.

43. The host cell of claim 42, which is a mammalian host cell.

44. A method for producing an antibody set forth in any one of claims 1-39, comprising:
(i) culturing the host cell of claim 42 or claim 43 under conditions allowing for expression of the antibody; and
(ii) harvesting the antibody thus produced.

45. A pharmaceutical composition, comprising an antibody or bi-specific antibody set forth in any one of claims 1-39, or a nucleic acid(s) encoding such, and a pharmaceutically acceptable carrier.

46. A method for modulating immune responses, comprising administering an effective amount of the antibody of any one of claims 1-39, a nucleic acid(s) encoding such, or a pharmaceutical composition comprising the antibody or encoding nucleic acid(s) to a subject in need thereof.

47. The method of claim 46, wherein the subject is a human patient having or suspected of having cancer.

48. The pharmaceutical composition of claim 45, which is for use in treating cancer in a subject, who optionally is a human cancer patient.
